(19) United States
(12) Patent Application Publication
(10) Pub. No.: US 2025/0101013 A1
(43) Pub. Date: Mar. 27, 2025

(54) FUNCTIONALIZED AMINOTHIAZOLES

(71) Applicant: LEADXPRO AG, Villigen (CH)

(72) Inventors: Werner NEIDHART, Villigen (CH); Denis BUCHER, Villigen (CH)

(21) Appl. No.: 18/728,675

(22) PCT Filed: Jan. 18, 2023
(86) PCT No.: PCT/EP2023/051147

§ 371 Date: Jul. 12, 2024

(30) Foreign Application Priority Data
Jan. 19, 2022 (EP) 22152325.1
Mar. 17, 2022 (EP) 22162769.8

(51) Int. Cl. C07D 417/14; A61K 31/427; A61K 31/4439; A61K 31/501; A61K 31/506

(52) U.S. Cl. CPC C07D 417/14; A61K 31/427; A61K 31/4439; A61K 31/501; A61K 31/506

(57) ABSTRACT

The present invention relates to novel antagonists of the A2B adenosine receptor and pharmaceutical compositions comprising said antagonists as well as their uses for the treatment and prevention of disorders known to be susceptible to improvement by antagonism of the A2B receptor such as asthma, chronic obstructive pulmonary disorder (COPD), pulmonary fibrosis, vascular diseases, allergic diseases, hypertension, retinopathy, diabetes mellitus, inflammatory gastrointestinal tract disorders, inflammatory diseases, autoimmune diseases, renal diseases, neurological disorders and, in particular, cancers. In particular, the present invention relates to compounds of Formula (I) wherein: R¹ is H, halogen, —C₁-C₈ alkyl, —C₃-C₈ cycloalkyl, —O—C₁-C₈ alkyl, or —CN, wherein each alkyl, cycloalkyl, or —O-alkyl is optionally substituted with one or more R²; R² is independently, at each occurrence, selected from halogen, —OH, and —OC₁-C₈ alkyl; R^{A} is a 5- to 6-membered heteroaryl, wherein said heteroaryl is optionally substituted with one or more R^{A1}; R^{A1} is independently, at each occurrence, selected from halogen, —OH, oxo, cyano, —C₁-C₈ alkyl, —C₃-C₈ cycloalkyl, and —O—C₁-C₈ alkyl, wherein each alkyl, cycloalkyl, or —O-alkyl is optionally substituted with one or more R^{A2}; R^{A2} is independently, at each occurrence, halogen, —OC₁-C₈ alkyl, or —OH; R^{B} is phenyl or a 5- to 6-membered heteroaryl, wherein each phenyl or heteroaryl is optionally substituted with one or more R^{B1}; and R^{B1} is independently, at each occurrence, selected from halogen, —OH, cyano, —C₁-C₈alkyl, and —O—C₁-C₈ alkyl, wherein each alkyl and —O-alkyl is optionally substituted with one or more R^{B2}; and R^{B2} is independently, at each occurrence, halogen, —OH, or —OC₁-C₈ alkyl; or a pharmaceutically acceptable salt, solvate, or hydrate thereof.

The present invention relates to selective A_{2B} antagonists and dual A_{2B} and A₁ antagonists with selectivity over other adenosine receptor subtypes (e.g., A_{2A} and/or A₃). The inventive compounds are useful in the treatment and prophylaxis of disorders known to be treatable by antagonism of the adenosine A₁ and/or A_{2B} receptors (particularly the A_{2B} receptor). Such disorders include asthma, chronic obstructive pulmonary disorder (COPD), pulmonary fibrosis, vascular diseases, allergic diseases, hypertension, retinopathy, diabetes mellitus, inflammatory gastrointestinal tract disorders, inflammatory diseases, autoimmune diseases, renal diseases, neurological disorders, otological disorders, and in particular cancer.

### RELATED ART

Adenosine is an endogenous modulator of multiple physiological responses that play an important role in protecting tissues during injury and acute states such as hypoxia, inflammation and ischemia. These responses include for instance vasodilation, pain, anti-inflammation, wound healing and thermogenesis. In contrast to its beneficial effects during acute states, chronic exposure to adenosine and its associated physiological responses can be detrimental and initiate or prolong diseases such as hypertension, pulmonary and renal fibrosis and cancer (Gessi et al., Expert Opin. Investig. Drugs, (2011) 20(12):1591-609). In the immune system, adenosine is a central mediator impacting most if not all immune cells and affecting many aspects of immune responses. These immunoregulatory effects, which are mostly anti-inflammatory, contribute to the general tissue protective effects of adenosine and its receptors. However, in some instances, the effect of adenosine on the immune system is deleterious, as prolonged signaling can hinder anti-tumor and antibacterial immunity, thereby promoting cancer development and progression and sepsis, respectively. The action of adenosine is transmitted by adenosine receptors that belong to the G-coupled signaling receptors and are broadly expressed in normal tissues in 4 subtypes (A₁, A_{2A}, A_{2B}, A₃). Adenosine receptors couple to different types of G proteins and mediate various signaling pathways in cells.

Cancer therapy, in particular, could benefit from compounds that target adenosine-dependent pathways selectively, as adenosine signaling plays roles both in cancer development and immune evasion (Ohta et al., Proc. Natl. Acad. Sci. USA, (2006) 103(35):13132-7); Kasama et al., BMC Cancer, (2015) 15(563); Corona et al., J. Cancer Metastasis Treat. (2017) 3:206-08; Allard et al., Immunol. Cell Biol. (2017) 95(4): 333-339). In cancer cells, adenosine signaling has been shown to regulate apoptosis, angiogenesis and metastasis (Bahreyni et al., J. Cell Physiol. (2018) 233(3):1836-1843). Furthermore, adenosine antagonism in vivo has been shown to reverse immunosuppressive phenotypes in the tumor microenvironment. As such, adenosine is considered a negative immune checkpoint that can be deregulated in cancer and create an immunosuppressive tumor microenvironment responsible for cancer resistance and aggressiveness. Studies in mice have shown that antagonism of adenosine signaling could restore the immune response and result in durable antitumor responses (Ohta et al., Proc. Natl. Acad. Sci. USA, (2006) 103(35):13132-7). Moreover, the anti-tumor effect was shown to be largely mediated by T cells, as shown by CXCR3−/− knock-out mice that displayed loss of sensitivity to adenosine antagonists (Cekic et al., J. Immunol. (2012) 188(1):198-205). Although inhibition of the A_{2A} receptor subtype has to date elicited the most attention, A_{2A} signaling is known to display contradictory effects on cell growth, as it can improve cell proliferation, but may also induce apoptosis and impair cell survival Merighi et al., J. Invest. Dermatol. (2002) 119(4):923-33; Wei et al., Purinergic Signal. (2013) 9(2):271-80). For this reason, the blockade of the A_{2A} pathway may in some cases be counter-productive, as it blocks the A_{2A} apoptosis signal leading to increased cell proliferation. For instance in a caco2 cancer cell line, adenosine decreased cell proliferation at high dose—due to the apoptosis mechanism dominating over the cell growth component—and this effect was reversed only by A_{2A} antagonist but not any other subtype antagonists (Yasuda et al., J. Gastroenterol. (2009) 56-65). This led some authors to label the strategy of dual compounds targeting both A_{2A} and A_{2B} subtypes a “double-edge sword” (Allard et al., Curr. Opin. Pharmacol. (2016) 29:7-16), supporting the need for alternatives such as selective A_{2B} inhibitors. Indeed, the study of A_{2A}−/− and A_{2B}−/− knock-out mice suggested that the nonselective compound theophylline had a beneficial effect on tumor growth in vivo via A_{2B} antagonism. Instead, tumor grew faster in the A_{2A} knockouts than in the wild-type mice (Cekic et al., J Immunol. (2012) 188(1):198-205), providing a strong rationale for the development of antagonists with specific A_{2B} selectivity versus A_{2A}.

Selectivity versus the A₃ receptor subtype is another important feature of drugs used to target adenosine receptors. To date several pieces of evidence support the conclusion that activation of A₃R signaling is crucial for cardio protection during and following ischemia-reperfusion and it has been proposed that a consistent part of the cardioprotective effects exerted by adenosine, once largely attributed to the A₁ receptor, may now be in part ascribed to A₃R activation (Headric and Peart, Vascul. Pharmacol. (2005) 42(5-6):271-9). A₃ receptor antagonism has been linked to tissue damage following ischemia in the CNS, heart, kidney, lung, eye, potentially deleterious effects on motor function and pain thresholds (Gessi et al., Pharmacol Ther. (2008) 117(1):123-40).

Although a strong rationale exists for targeting the A_{2B} receptor with antagonists, and the A_{2B} receptor is found in many different cellular types, it has traditionally elicited less interest than the A₁, A_{2A}, and A₃ subtypes due to its low affinity for adenosine. However, it is now known that the extracellular concentration of adenosine increases significantly in the hypoxic tumor microenvironment and can lead to the activation of A_{2B} receptors (Kasama et al., BMC Cancer (2015) 15, 563). In recent years, a link between A_{2B} and cancer has emerged, and the tumor-promoting activity of A_{2B} signaling could be deduced from A_{2B}-deficient mice, where tumor growth was decreased compared with wild-type counterparts. This effect was in part attributed to a significant decrease in the intratumoral levels of vascular endothelial growth factor (VEGF) (Rhzhov et al., Neoplasia. (2008) 10(9):987-95). In addition, A_{2B} was found to induce tumor growth by producing basic fibroblast growth factor (bFGF) in lung, colon, and prostate cancers (Bahreyni et al., J. Cell Physiol. (2018) 233(3):1836-1843). A_{2B} was shown to play a role in supporting invasion and metastatic spreading by the accumulation of non-prenylated Rap 1B, a small GTPase controlling cell adhesion (Ntanie et al., Sci. Signal. (2013) 6(277):ra39). Additionally, A_{2B} plays a central role in the inflammatory response to the tumor, such as the amount of tumor-infiltrating myeloid-derived suppressor cells (MDSCs) (Ryzhov et al., Neoplasia. (2008) 10(9):987-95). Moreover, A_{2B} is involved in the maturation of dendritic cells (DCs) and macrophages differentiation and function, and as such, is crucial for tumor immune surveillance (Iannone et al., Neoplasia. (2013) 15(12):1400-9; Allard et al., Immunol. Cell Biol. (2017) 95(4):333-339). Therefore, the anti-cancer benefits of A_{2B} antagonists are diverse and occur both via tumor-intrinsic and host-mediated pathways. High A_{2B} expression has been associated with poor prognosis in several cancers (Ihara et al., Oncogene. (2017) 36(14):2023-2029), and was found to be increased compared to the adjacent normal tissues in diverse human cancers including ovarian, lung, liver, oral, colon and prostate cancers (Zhou et al., Oncotarget. (2017) 8(30):48755-48768). In oral squamous cell carcinoma, the A_{2B} receptor was overexpressed and its silencing inhibited growth (Kasama et al., BMC Cancer, (2015) 15(563). The use of the selective A_{2B} agonist BAY 60-6583 enhanced melanoma progression in mice, while the selective A_{2B} antagonist PSB1115 suppressed melanoma growth (Iannone et al., Neoplasia. (2013) 15(12):1400-9). Furthermore, the combination of PSB1115 with other checkpoint inhibitors used in cancer immunotherapy, such as PD-1 or CTLA-4 inhibitors, showed that A_{2B} antagonism displays strong synergies with these compounds in vivo, confirming that they proceed via a fundamentally different mechanism (Mittal et al., Cancer Res. (2016) 76(15):4372-82). Numerous A_{2B} antagonists are currently in development but none has yet received regulatory approval.

A₁ receptor antagonism is also believed to show strong potential for cancer therapy. The A₁ receptor has been reported to be involved in the pathogenesis of various cancers, including breast, colon, astrocytoma, renal, and gastric cancers (Sai et al., Neurotoxicology. (2006) 27(4):458-67; Hosseinzadeh et al., Iran Biomed J. (2008) 12(4):203-8; Saito et al., Cancer Lett. (2010) 28; 290(2):211-5; Sheng et al., Front. Biosci. (Landmark Ed). (2014) 19:854-61). Like A_{2B}, the anti-cancer benefits of A₁ antagonists are diverse and occur both via tumor-intrinsic and host-mediated pathways. For instance, A₁ receptor antagonist DPCPX was shown to inhibit tumor progression in renal cell carcinoma (Zhou et al., Cell Physiol. Biochem. (2017) 43(2):733-742). DPCPX also induced apoptosis in MCF-7 breast cancer cells (Dastjerdi et al., Bratisl. Lek. Listy. (2016) 117(4):242-6), and upregulation of p53. In line with this behavior, RNA silencing of A₁ receptor in overexpressing breast carcinoma cells lead to diminished rates of cell proliferation and induction of apoptosis (Mirza et al., Cancer Biol. Ther. (2005) 4(12):1355-60).

An additional rationale for A₁ receptor antagonism also exists at the host level, which is most relevant for cancer immunotherapy. Studies have found that pharmacological preconditioning with A₁ agonists can lead to the upregulation of A_{2A} receptor and trigger the immunosuppressing action expected for A_{2A} signaling (Nakav et al., PLoS ONE 3(5): e2107); (Nakav et al., Nephrology Dialysis Transplantation, (2009) 24(8) 2392-2399); (Naamani et al., International Immunopharmacology, (2014) 20(1) 205-212); This effect was later replicated in vivo and found to induce immunosuppression leading to improved graft survival in allogeneic transplantation models (Naamani et al., Sci. Rep. (2020) 11; 10(1):4464). For cancer, targeting A₁ signaling could have a positive effect in the high adenosine concentration of the tumor microenvironment, where upregulation of A_{2A} receptor would lead to immune suppression and tumor evasion. Furthermore, A₁ receptor antagonism is potentially beneficial outside cancer therapy as it is also being studied, for instance, in the treatment of respiratory diseases such as asthma and obstructive pulmonary disease (COPD).

A_{2B} receptor antagonism can also be applied in fields outside immunooncology, particularly for the treatment of respiratory diseases, where A_{2B} signaling mediates the production and release of pro-inflammatory mediators from mast cells (e.g., IL-4, IL-8, IL-13, and histamine). Mice treated with A_{2B} receptor antagonists have less pulmonary inflammation, less fibrosis and greater alveolar airspace enlargement than non-treated mice, demonstrating the potential of A_{2B} antagonists for reducing pulmonary inflammation in vivo (Sun et al., J Clin Invest. (2006) 116(8):2173-2182). Other indications include pulmonary fibrosis, pulmonary hypertension (PH), COPD, and asthma (Zablocki et al., Expert Opinion on Therapeutic Patents, (2006) 16:10, 1347-1357). Consistent with its anti-inflammatory and immunosuppressive effects, A_{2B} has been found to be implicated in different aspects of glucose regulation. A_{2B} antagonists were found to decrease the inflammatory response and improve insulin resistance in a diabetic mouse strain by attenuating the production of IL-6 and other cytokines that influence glucose and fat metabolism (Figler et al., Diabetes. (2011) 60(2) 669-79). In addition, A_{2B} antagonists proved to be able to prevent fatty liver formation post alcohol consumption in mice models (Peng et al., J Clin. Invest. (2009) 119(3):582-94). Studies on mouse models have shown that A_{2B} inhibition can protect against induced diabetic nephropathy and renal fibrosis. In addition, renal biopsy samples from patients and genetic and pharmacological approaches also support a potential role for A_{2B} inhibition in the treatment of chronic kidney disease (CKD) and renal ischemia (Sun and Huang, Front Chem. (2016) 24; 4:37). The role of A_{2B} antagonists in the central nervous system has attracted less attention than A_{2A} inhibition. However, A_{2B} is closely related to A_{2A} receptors that have shown clear antiparkinsonian effects and are of great interest exists in Alzheimer's disease, brain ischaemia, spinal cord injury, drug addiction and other conditions. Manalo has reported that A_{2B}-specific antagonist treatment in Ada^{-/-} mice significantly improved hearing loss, nerve fiber density, and myelin compaction in mice, and that the A2B receptor is a pathogenic factor underlying age-related sensorineural hearing loss (Manalo et al., FASEB J. (2020) 34(12) 15771-15787). The low affinity of A_{2B} receptors for adenosine implies that they might represent a good therapeutic target, since these receptors are activated only under pathological conditions when adenosine levels raise up to micromolar concentrations (Popoli and Pepponi, CNS Neurol Disord Drug Targets. (2012) 11(6):664-74). Thus there is a need for safe and selective ligands for A_{2B} receptors, and/or A1 receptors for the treatment of a broad range of diseases and disorders including cancer and inflammatory diseases.

### SUMMARY OF THE INVENTION

The present invention provides selective A_{2B} antagonists and dual A_{2B} and A₁ antagonists with selectivity over other adenosine receptor subtypes (e.g., A_{2A} and A₃). The invention pertains to compounds of Formula I or pharmaceutically acceptable salts or hydrates thereof, and their use as therapeutically active substances for the treatment or prevention of conditions, disorders or diseases, in particular in the treatment or prevention of cancer.

More specifically, the present invention pertains to the discovery of certain 2-aminothiazoles bearing a triazole substituent at the 2-amino group. The compounds are useful as A₁ and A_{2B} receptor antagonists, particularly as A_{2B} receptor antagonists, and do not exhibit meaningful, e.g., medically relevant A₃ adenosine receptor antagonistic activity. Thus, the compounds of the present invention particularly suited as new medicines for the treatment of disease.

The selectivity profile of the compounds of the invention enables their use as new medicines, especially for the treatment of cancer, because both A₁ and A_{2B} receptors have been shown to constitute important regulators of cancer propagation and immune cancer response in the tumor microenvironment as described above. Further, an important feature of the invention is the high selectivity compared to A₃ receptor antagonistic activity, which in preferred embodiments prevents unwanted side-effects of the inventive compounds caused by antagonism of the A₃ receptor.

Moreover, the molecules of the invention comprise a triazole substituent bonded at the 2-amino group of the thiazole core, which further imparts favorable properties for drug candidates such as solubility, lipophilicity, and cell permeation. For example, the aminotriazole group of compounds of Formula I is hydrophilic, and in some preferred embodiments balances the lipophilicity of the thiazole group and associated R^{A} and R^{B} substituents to afford compounds with improved cell membrane permeation. Moreover, the aminotriazole group incorporates a polar NH group that can enhance solubility while maintaining acceptable cell permeation through possible formation of an intramolecular hydrogen bond via the nitrogen of the central thiazole.

Thus, the inventive compounds disclosed herein are particularly suited as therapeutics. Additional features and advantages of the present technology will be apparent to one of skill in the art upon reading the Detailed Description, below.

Thus, in a first aspect, the present invention provides compounds of Formula I:

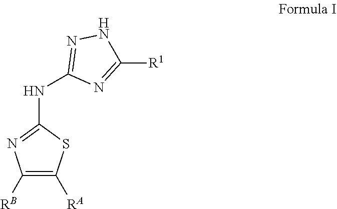

wherein:
R¹ is H, halogen, —C₁-C₈ alkyl, —C₃-C₈ cycloalkyl, —O—C₁-C₈ alkyl, or —CN, wherein each alkyl, cycloalkyl, or —O-alkyl is optionally substituted with one or more R²;
R² is independently, at each occurrence, selected from halogen, —OH, and —OC₁-C₈ alkyl;
R^{A} is a 5- to 6-membered heteroaryl, wherein said heteroaryl is optionally substituted with one or more R^{A1};
R^{A1} is independently, at each occurrence, selected from halogen, —OH, oxo, cyano, —C₁-C₈ alkyl, —C₃-C₈ cycloalkyl, and —O—C₁-C₈ alkyl, wherein each alkyl, cycloalkyl, or —O-alkyl is optionally substituted with one or more R^{A2};
R^{A2} is independently, at each occurrence, halogen, —OC₁-C₈ alkyl, or —OH;
R^{B} is phenyl or a 5- to 6-membered heteroaryl, wherein each phenyl or heteroaryl is optionally substituted with one or more R^{B1}; and
R^{B1} is independently, at each occurrence, selected from halogen, —OH, cyano, —C₁-C₈ alkyl, and —O—C₁-C₈ alkyl, wherein each alkyl and —O-alkyl is optionally substituted with one or more R^{B2}; and
R^{B2} is independently, at each occurrence, halogen, —OH, or —OC₁-C₈ alkyl;
or pharmaceutically acceptable salts, solvates, or hydrates thereof.

In another aspect, the present invention provides the inventive compound of Formula I for use as a medicament.

In another aspect, the present invention provides a pharmaceutical composition comprising the inventive compound of Formula I, optionally together with a pharmaceutically acceptable diluent or carrier.

In a further aspect, the present invention provides the inventive compound of Formula I or the inventive pharmaceutical composition for use in a method of treating a condition, disorder or disease mediated by activation of the adenosine A_{2B} and/or A₁ receptor, preferably the A_{2B} receptor.

In a further aspect, the present invention provides the inventive compound of Formula I or the inventive pharmaceutical composition for use in a method of treating a condition, disorder or disease ameliorated by the inhibition of the adenosine A_{2B} and/or A₁ receptor, preferably the A_{2B} receptor.

In a further aspect, the present invention provides the inventive compound of Formula I or the inventive pharmaceutical composition for use in a method of treating a condition or disease susceptible to amelioration by antagonism of the adenosine A_{2B} and/or A₁ receptor, preferably the A_{2B} receptor.

In another aspect, the present invention provides the use of a compound of Formula I according to the present invention in the manufacture of a medicament for the treatment of a condition, disorder or disease mediated by activation of the adenosine A_{2B} and/or A₁ receptor, preferably the A_{2B} receptor.

In another aspect, the present invention provides the use of a compound of Formula I according to the present invention in the manufacture of a medicament for the treatment of a condition, disorder or disease ameliorated by the inhibition of the adenosine A_{2B} and/or A₁ receptor, preferably the A_{2B} receptor.

In another aspect, the present invention provides the use of a compound of Formula I according to the present invention in the manufacture of a medicament for the treatment of a condition, disorder or disease susceptible to amelioration by antagonism of the adenosine A_{2B} and/or A₁ receptor, preferably the A_{2B} receptor.

In another aspect, the present invention provides a method of treating a condition, disorder or disease mediated by activation of the adenosine A_{2B} and/or A₁ receptor, preferably the A_{2B} receptor in a subject in need thereof, the method comprising administering to the subject a therapeutically effective amount of a compound of Formula I according to the present invention.

In another aspect, the present invention provides a method of treating a condition, disorder or disease ameliorated by the inhibition of the adenosine A_{2B} and/or A₁ receptor, preferably the A_{2B} receptor in a subject in need thereof, comprising administering to the subject a therapeutically effective amount of a compound of Formula I according to the present invention.

In another aspect, the present invention provides a method of treating a condition, disorder or disease susceptible to amelioration by antagonism of the adenosine A_{2B} and/or A₁ receptor, preferably the A_{2B} receptor in a subject in need thereof, comprising administering to the subject a therapeutically effective amount of a compound of Formula I according to the present invention.

In another aspect, the present invention provides the inventive compound of Formula I or the inventive pharmaceutical composition for use in a method of treating a condition, disorder or disease selected from a respiratory disease, an inflammatory obstructive airway disease, an inflammatory disease, a metabolic disease, a renal disease, a vascular disease, an allergic disease, an inflammatory gastrointestinal tract disorder, an autoimmune disease, a neurological disorder, an otological disorder, and a cancer.

In another aspect, the present invention provides the use of a compound of Formula I according to the present invention in the manufacture of a medicament for the treatment of a condition, disorder or disease selected from a respiratory disease, an inflammatory obstructive airway disease, an inflammatory disease, a metabolic disease, a renal disease, a vascular disease, an allergic disease, an inflammatory gastrointestinal tract disorder, an autoimmune disease, a neurological disorder, an otological disorder, and a cancer.

In another aspect, the present invention provides a method of treating a condition, disorder or disease selected from a respiratory disease, an inflammatory obstructive airway disease, an inflammatory disease, a metabolic disease, a renal disease, a vascular disease, an allergic disease, an inflammatory gastrointestinal tract disorder, an autoimmune disease, a neurological disorder, an otological disorder, and a cancer, wherein said method comprises administering to a subject, particularly a human subject, in need thereof, a therapeutically effective amount of an inventive compound of Formula I or a pharmaceutically acceptable salt, or hydrate thereof.

Moreover, the present invention provides processes for the manufacture of said compounds, intermediates as well as pharmaceutical compositions and medicaments containing said compounds or pharmaceutically acceptable salts, or hydrates thereof and, in addition, uses of the same for methods of prevention or treatment of disorders and diseases mediated by activation of A₁ and, particularly, adenosine A_{2B} receptors.

Further aspects and embodiments of the present invention will be become apparent as this description continues.

### DETAILED DESCRIPTION OF THE INVENTION

### Definitions

Unless defined otherwise, all technical and scientific terms used herein have the same meanings as commonly understood by one of ordinary skill in the art to which this invention belongs. The definitions used below typically refer to fragments by specifying a number of carbon atoms, for example a “C₁-C₈—” fragment. Such usage is for definition purposes and is not intended to be limited to fragments with the particular number of carbon atoms stated. The definitions for fragments with lower or higher numbers of carbon atoms, for example a “C₁-C₄—” or a “C₁-C₃—” analogue, are to be interpreted as identical with the definition of the larger “C₁-C₈—” fragment except for the number of carbon atoms.

“C₁-C₈ alkyl”, as used herein, refers to straight chain or branched saturated C₁-C₈ hydrocarbon, which may be, for example, methyl, ethyl, n-propyl, isopropyl, n-butyl, isobutyl, sec-butyl, tert-butyl, straight or branched pentyl, straight or branched hexyl, straight or branched heptyl, or straight or branched octyl. Preferably, C₁-C₈ alkyl is C₁-C₄ alkyl.

“C₁-C₈ alkoxy”, as used herein, refers to straight chain or branched saturated —O—C₁-C₈ hydrocarbon which may be, for example, methoxy, ethoxy, n-propoxy, isopropoxy, n-butoxy, isobutoxy, sec-butoxy, tert-butoxy, straight or branched pentoxy, straight or branched hexyloxy, straight or branched heptyloxy, or straight or branched octyloxy. Preferably, C₁-C₈ alkoxy is C₁-C₄ alkoxy. A “C₁-C₈ alkoxy” group can also be represented as “—O—C₁-C₈ alkyl.”

“Halogen”, as used herein, refers to fluorine, chlorine, bromine or iodine; preferably it is fluorine or chlorine, more preferably it is fluorine or chlorine when incorporated in a compound of Formula I. This also applies, correspondingly, to halogen in combination with other meanings, such as haloalkyl.

“C₁-C₈ haloalkyl”, as used herein, refers to C₁-C₈ alkyl as hereinbefore defined substituted by one or more halogen atoms, preferably one, two or three halogen atoms, preferably fluorine or chlorine atoms. Preferably, C₁-C₈ haloalkyl is C₁-C₄ alkyl substituted by one, two or three fluorine or chlorine atoms. Preferred examples include difluoromethyl, trifluoromethyl, chlorodifluoromethyl and 2,2,2-trifluoro-ethyl.

“C₁-C₈ hydroxyalkyl” as used herein, refers to C₁-C₈ alkyl as hereinbefore defined substituted by one or more hydroxyl (OH) groups, preferably one, two or three hydroxyl (OH) groups, most preferably one hydroxyl (OH) group.

The term “C₃-C₈ cycloalkyl”, as used herein, refers to a mono- or bi-cyclic saturated hydrocarbon ring, typically and preferably to a mono-cyclic form, and containing 3 to 8 carbon atoms, more preferably 3 to 7 carbon atoms. Specific and preferred examples of monocyclic cycloalkyl groups include cyclopropyl, cyclobutyl and cyclohexyl. The term “C₃-C₆ cycloalkyl”, as used herein, refers to a monocyclic form containing 3 to 6 carbon atoms and specifically to cyclopropyl, cyclobutyl, cyclopentyl or cyclohexyl.

The term “phenyl” as used herein, refers to a —C₆ aromatic ring. An unsubstituted phenyl group has the formula —C₆H₅. A substituted phenyl group has one or more hydrogen atoms replaced by a substituent.

The term “heteroaryl”, as used herein, refers to a 5- to 6-membered ring system comprising an aromatic ring and containing at least one heteroatom selected from O and N. A heteroaryl may be a single ring or two or more fused rings wherein at least one ring contains a heteroatom. Examples of monocyclic heteroaryl include imidazolyl, triazolyl, isoxazolyl, oxazolyl, pyrazolyl, pyridinyl, pyridazinyl, pyrimidinyl, and pyrazinyl.

The term “cyano” refers to the group —CN.

The term “carbonyl” refers to a functional group composing a carbon atom double-bonded to an oxygen atom. It can be abbreviated herein as “oxo”, as C(O), or as C═O.

Where a group is said to be “optionally substituted,” preferably there are optionally 1-5 substituents, more preferably optionally 1-3 substituents, again more preferably optionally 1 or 2 substituents, and most preferably optionally 1 substituent. Where a group is said to be optionally substituted and where there are more than one substituents for said optional substitution of said group, said more than one substituents can either be the same or different.

The term “treating”, “treatment” or “therapy” as used herein refers to means of obtaining a desired physiological effect. The effect may be therapeutic in terms of partially or completely curing a disease or a condition and/or symptoms attributed to the disease or the condition. The term refers to inhibiting the disease or condition, i.e. arresting its development; or ameliorating the disease or condition, i.e. causing regression of the disease or condition.

As used herein, the term “for use” as used in “composition for use in treatment of a disease” shall disclose also the corresponding method of treatment and the corresponding use of a preparation for the manufacture of a medicament for the treatment of a disease.

A “therapeutically effective amount” is the amount of a compound or pharmaceutical composition in accordance with the present invention that will elicit the biological or medical response of a subject, preferably a human subject that is being sought by the researcher, veterinarian, medical doctor or other clinician. The term “therapeutic administration”, as used herein, refers to the administration of therapeutically effective amount.

### Compounds of the Invention

In a first aspect, the present invention provides compounds of Formula I:

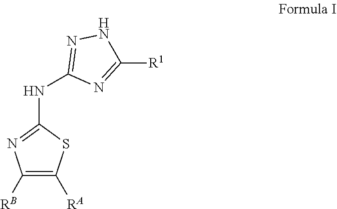

wherein:
R¹ is H, halogen, —C₁-C₈ alkyl, —C₃-C₈ cycloalkyl, —O—C₁-C₈ alkyl, or —CN, wherein each alkyl, cycloalkyl, or —O-alkyl is optionally substituted with one or more R²;
R² is independently, at each occurrence, selected from halogen, —OH, and —OC₁-C₈ alkyl;
R^{A} is a 5- to 6-membered heteroaryl, wherein said heteroaryl is optionally substituted with one or more R^{A1};
R^{A1} is independently, at each occurrence, selected from halogen, —OH, oxo, cyano, —C₁-C₈ alkyl, —C₃-C₈ cycloalkyl, and —O—C₁-C₈ alkyl, wherein each alkyl, cycloalkyl, or —O-alkyl is optionally substituted with one or more R^{A2};
R^{A2} is independently, at each occurrence, halogen, —OC₁-C₈ alkyl, or —OH;
R^{B} is phenyl or a 5- to 6-membered heteroaryl, wherein each phenyl or heteroaryl is optionally substituted with one or more R^{B1}; and
R^{B1} is independently, at each occurrence, selected from halogen, —OH, cyano, —C₁-C₈ alkyl, and —O—C₁-C₈ alkyl, wherein each alkyl and —O-alkyl is optionally substituted with one or more R^{B2}; and
R^{B2} is independently, at each occurrence, halogen, —OH, or —OC₁-C₈ alkyl;
or pharmaceutically acceptable salts, solvates, or hydrates thereof.

In some embodiments, R¹ is H, halogen, —C₁-C₈ alkyl, —C₃-C₈ cycloalkyl, or —OC₁-C₈ alkyl, wherein each alkyl, cycloalkyl, or —O-alkyl is optionally substituted with one or more R².

In a further preferred embodiment, R¹ is H, halogen, —C₁-C₈ alkyl, or —C₃-C₈ cycloalkyl, wherein each alkyl or cycloalkyl is optionally substituted with one or more R².

In a further preferred embodiment, R¹ is H, halogen, —C₁-C₄ alkyl, or —C₃-C₆ cycloalkyl, wherein each alkyl or cycloalkyl is optionally substituted with one or more R².

In a further preferred embodiment, R¹ is H, halogen, —C₁-C₂ alkyl, or —C₃-C₆ cycloalkyl, wherein each alkyl or cycloalkyl is optionally substituted with one or more R².

In a further preferred embodiment, R¹ is H, halogen, —C₁-C₂ alkyl, or —C₃-C₄ cycloalkyl, wherein each alkyl or cycloalkyl is optionally substituted with one or more R².

In a further preferred embodiment, R¹ is H, halogen, —C₁ alkyl, or —C₃-C₄ cycloalkyl, wherein each alkyl or cycloalkyl is optionally substituted with one or more R².

In a further preferred embodiment, R¹ is H, fluoro, chloro, —C₁-C₂ alkyl, or —C₃-C₄ cycloalkyl, wherein each alkyl or cycloalkyl is optionally substituted with one or two R².

In a further preferred embodiment, R¹ is H, fluoro, chloro, —C₁ alkyl, or —C₃-C₄ cycloalkyl, wherein each alkyl or cycloalkyl is optionally substituted with one or two R².

In a further preferred embodiment, R¹ is H, fluoro, chloro, —C₁-C₂ alkyl, or —C₃-C₄ cycloalkyl, wherein each alkyl or cycloalkyl is optionally substituted with one R².

In a further preferred embodiment, R¹ is H, fluoro, chloro, —C₁ alkyl, or —C₃-C₄ cycloalkyl, wherein each alkyl or cycloalkyl is optionally substituted with one R².

In a further preferred embodiment, R¹ is H, —C₁-C₂ alkyl, or —C₃-C₄ cycloalkyl, wherein each alkyl is optionally substituted with one R².

In a further preferred embodiment, R¹ is H, —C₁ alkyl, or —C₃-C₄ cycloalkyl, wherein each alkyl is optionally substituted with one R².

In a further preferred embodiment, R¹ is H, —C₁-C₂ alkyl, or —C₃-C₄ cycloalkyl, wherein each alkyl is optionally substituted with one —OH.

In a further preferred embodiment, R¹ is H, —C₁ alkyl, or —C₃-C₄ cycloalkyl, wherein each alkyl is optionally substituted with one —OH.

In a further preferred embodiment, R¹ is H, —CH₃, —CH₂CH₃, —CH₂OH, or —C₃-C₄ cycloalkyl.

In a further preferred embodiment, R¹ is H, —CH₂OH, or —C₃-C₄ cycloalkyl.

In a further preferred embodiment, R¹ is H. In a further preferred embodiment, R¹ is halogen. In a further preferred embodiment, R¹ is fluoro or chloro.

In a further preferred embodiment, R¹ is —CH₃.

In a further preferred embodiment, R¹ is —CH₂CH₃.

In a further preferred embodiment, R¹ is —CH₂OH.

In a further preferred embodiment, R¹ is cyclopropane.

In a further preferred embodiment, R¹ is cyclobutane.

In some preferred embodiments, R² is —OH, halogen or —OC₁-C₄ alkyl. In a further preferred embodiment, R² is —OH, halogen, or —OC₁-C₂ alkyl. In a further preferred embodiment, R² is —OH or halogen, wherein said halogen is preferably fluoro or chloro. In a further preferred embodiment, R² is —OH. In a further preferred embodiment, R² is halogen, wherein said halogen is preferably fluoro or chloro.

In some preferred embodiments, R^{A} is imidazolyl, triazolyl, isoxazolyl, oxazolyl, pyrazolyl, pyridinyl, pyridazinyl, pyrimidinyl, pyrazinyl, or pyridonyl; wherein each imidazolyl, triazolyl, isoxazolyl, oxazolyl, pyrazolyl, pyridinyl, pyridazinyl, pyrimidinyl, pyrazinyl or pyridonyl is optionally substituted with one or more R^{A1}.

In some preferred embodiments, R^{A} is imidazolyl, triazolyl, isoxazolyl, oxazolyl, pyrazolyl, pyridinyl, pyridazinyl, pyrimidinyl, or pyrazinyl; wherein each imidazolyl, triazolyl, isoxazolyl, oxazolyl, pyrazolyl, pyridinyl, pyridazinyl, pyrimidinyl, or pyrazinyl is optionally substituted with one or more R^{A1}.

In a further preferred embodiment, R^{A} is 1-imidazolyl, 1-(1,2,4)-triazolyl, 4-(1,2,4)-triazolyl, 4-isoxazolyl, 5-isoxazolyl, 5-oxazolyl, 4-pyrazolyl, 4-pyridinyl, 4-pyridazinyl, 4-pyrimidinyl, 2-pyrazinyl, or 5-(2-pyridonyl); wherein each imidazolyl, triazolyl, isoxazolyl, oxazolyl, pyrazolyl, pyridinyl, pyridazinyl, pyrimidinyl, pyrazinyl or pyridonyl is optionally substituted with one or more R^{A1}.

In a further preferred embodiment, R^{A} is 1-imidazolyl, 1-(1,2,4)-triazolyl, 4-(1,2,4)-triazolyl, 4-isoxazolyl, 5-isoxazolyl, 5-oxazolyl, 4-pyrazolyl, 4-pyridinyl, 4-pyridazinyl, 4-pyrimidinyl, or 2-pyrazinyl; wherein each imidazolyl, triazolyl, isoxazolyl, oxazolyl, pyrazolyl, pyridinyl, pyridazinyl, pyrimidinyl, or pyrazinyl is optionally substituted with one or more R^{A1}.

In a further preferred embodiment, R^{A} is imidazolyl, triazolyl, isoxazolyl, pyrazolyl, pyridinyl, pyridazinyl, pyrimidinyl, or pyridonyl; wherein each imidazolyl, triazolyl, isoxazolyl, pyrazolyl, pyridinyl, pyridazinyl, pyrimidinyl or pyridonyl is optionally substituted with one or more R^{A1}.

In a further preferred embodiment, R^{A} is imidazolyl, triazolyl, isoxazolyl, pyrazolyl, pyridinyl, pyridazinyl, or pyrimidinyl; wherein each imidazolyl, triazolyl, isoxazolyl, pyrazolyl, pyridinyl, pyridazinyl, or pyrimidinyl is optionally substituted with one or more R^{A1}.

In a further preferred embodiment, R^{A} is 1-imidazolyl, 1-(1,2,4)-triazolyl, 4-(1,2,4)-triazolyl, 4-isoxazolyl, 5-isoxazolyl, 4-pyrazolyl, 4-pyridinyl, 4-pyridazinyl, 4-pyrimidinyl, or 5-(2-pyridonyl); wherein each imidazolyl, triazolyl, isoxazolyl, pyrazolyl, pyridinyl, pyridazinyl, pyrimidinyl, or pyridonyl is optionally substituted with one or more R^{A1}.

In a further preferred embodiment, R^{A} is 1-imidazolyl, 1-(1,2,4)-triazolyl, 4-(1,2,4)-triazolyl, 4-isoxazolyl, 5-isoxazolyl, 4-pyrazolyl, 4-pyridinyl, 4-pyridazinyl, or 4-pyrimidinyl; wherein each imidazolyl, triazolyl, isoxazolyl, pyrazolyl, pyridinyl, pyridazinyl, and pyrimidinyl is optionally substituted with one or more R^{A1}.

In a further preferred embodiment, R^{A} is 1-imidazolyl, 1-(1,2,4)-triazolyl, 4-(1,2,4)-triazolyl, 4-isoxazolyl, 5-isoxazolyl, 4-pyrazolyl, 4-pyridinyl, 4-pyridazinyl, 4-pyrimidinyl, or 5-(2-pyridonyl); wherein each imidazolyl, triazolyl, isoxazolyl, pyrazolyl, pyridinyl, pyridazinyl, pyrimidinyl or pyridonyl is optionally substituted with one or two R^{A1}.

In a further preferred embodiment, R^{A} is 1-imidazolyl, 1-(1,2,4)-triazolyl, 4-(1,2,4)-triazolyl, 4-isoxazolyl, 5-isoxazolyl, 4-pyrazolyl, 4-pyridinyl, 4-pyridazinyl, or 4-pyrimidinyl; wherein each imidazolyl, triazolyl, isoxazolyl, pyrazolyl, pyridinyl, pyridazinyl, and pyrimidinyl is optionally substituted with one or two R^{A1}.

In a further preferred embodiment, R^{A} is imidazolyl, triazolyl, pyridinyl, pyridazinyl, pyrimidinyl, or pyridonyl; wherein each imidazolyl, triazolyl, pyridinyl, pyridazinyl, pyrimidinyl, or pyridonyl is optionally substituted with one or more R^{A1}.

In a further preferred embodiment, R^{A} is imidazolyl, triazolyl, pyridinyl or pyridonyl; wherein each imidazolyl, triazolyl, pyridinyl or pyridonyl is optionally substituted with one or more R^{A1}.

In a further preferred embodiment, R^{A} is imidazolyl, triazolyl, or pyridinyl; wherein each imidazolyl, triazolyl, and pyridinyl is optionally substituted with one or more R^{A1}.

In a further preferred embodiment, R^{A} is 1-imidazolyl, 1-(1,2,4)-triazolyl, 4-(1,2,4)-triazolyl, 4-pyridinyl, 4-pyridazinyl, 4-pyrimidinyl, or 5-(2-pyridonyl); wherein each imidazolyl, triazolyl, pyridinyl, pyridazinyl, pyrimidinyl or pyridonyl is optionally substituted with one or more R^{A1}.

In a further preferred embodiment, R^{A} is 1-imidazolyl, 1-(1,2,4)-triazolyl, 4-pyridinyl, 4-pyridazinyl, 4-pyrimidinyl, or 5-(2-pyridonyl); wherein each imidazolyl, triazolyl, pyridinyl, pyridazinyl, pyrimidinyl, or pyridonyl is optionally substituted with one or more R^{A1}.

In a further preferred embodiment, R^{A} is 1-imidazolyl, 1-(1,2,4)-triazolyl, 4-(1,2,4)-triazolyl, 4-pyridinyl, or 5-(2-pyridonyl); wherein each imidazolyl, triazolyl, pyridinyl or pyridonyl is optionally substituted with one or more R^{A1}.

In a further preferred embodiment, R^{A} is 1-imidazolyl, 1-(1,2,4)-triazolyl, 4-(1,2,4)-triazolyl, or 4-pyridinyl; wherein each imidazolyl, triazolyl, and pyridinyl is optionally substituted with one or more R^{A1}.

In a further preferred embodiment, R^{A} is 1-imidazolyl, 1-(1,2,4)-triazolyl, 4-(1,2,4)-triazolyl, 4-pyridinyl, 4-pyridazinyl, 4-pyrimidinyl, or 5-(2-pyridonyl); wherein each imidazolyl, triazolyl, pyridinyl, pyridazinyl, pyrimidinyl, or pyridonyl is optionally substituted with one or two R^{A1}.

In a further preferred embodiment, R^{A} is 1-imidazolyl, 1-(1,2,4)-triazolyl, 4-pyridinyl, 4-pyridazinyl, 4-pyrimidinyl, or 5-(2-pyridonyl); wherein each imidazolyl, triazolyl, pyridinyl, pyridazinyl, pyrimidinyl or pyridonyl is optionally substituted with one or two R^{A1}.

In a further preferred embodiment, R^{A} is 1-imidazolyl, 1-(1,2,4)-triazolyl, 4-(1,2,4)-triazolyl, 4-pyridinyl, or 5-(2-pyridonyl); wherein each imidazolyl, triazolyl, pyridinyl, or pyridonyl is optionally substituted with one or two R^{A1}.

In a further preferred embodiment, R^{A} is 1-imidazolyl, 1-(1,2,4)-triazolyl, 4-(1,2,4)-triazolyl, or 4-pyridinyl; wherein each imidazolyl, triazolyl, and pyridinyl is optionally substituted with one or two R^{A1}.

In a further preferred embodiment, R^{A} is 1-imidazolyl, 1-(1,2,4)-triazolyl, 4-(1,2,4)-triazolyl, 4-pyridinyl, 4-pyridazinyl, 4-pyrimidinyl, or 5-(2-pyridonyl); wherein each imidazolyl, triazolyl, pyridinyl, pyridazinyl, pyrimidinyl, or pyridonyl is unsubstituted.

In a further preferred embodiment, R^{A} is 1-imidazolyl, 1-(1,2,4)-triazolyl, 4-(1,2,4)-triazolyl, or 4-pyridinyl; wherein each imidazolyl, triazolyl, and pyridinyl is unsubstituted.

In a further preferred embodiment, R^{A} is 1-imidazolyl; wherein each imidazolyl is optionally substituted with one or two R^{A1}.

In a further preferred embodiment, R^{A} is 1-imidazolyl; wherein each imidazolyl is unsubstituted.

In a further preferred embodiment, R^{A} is 1-(1,2,4)-triazolyl; wherein each triazolyl is optionally substituted with one or two R^{A1}.

In a further preferred embodiment, R^{A} is 1-(1,2,4)-triazolyl; wherein each triazolyl is unsubstituted.

In a further preferred embodiment, R^{A} is 4-(1,2,4)-triazolyl; wherein each triazolyl is optionally substituted with one or two R^{A1}.

In a further preferred embodiment, R^{A} is 4-(1,2,4)-triazolyl; wherein each triazolyl is unsubstituted.

In a further preferred embodiment, R^{A} is 4-pyridinyl; wherein each pyridinyl is optionally substituted with one or two R^{A1}.

In a further preferred embodiment, R^{A} is 4-pyridinyl; wherein each pyridinyl is unsubstituted.

In a further preferred embodiment, R^{A} is 4-pyridazinyl; wherein each pyridazinyl is optionally substituted with one or two R^{A1}.

In a further preferred embodiment, R^{A} is 4-pyridazinyl; wherein each pyridazinyl is unsubstituted.

In a further preferred embodiment, R^{A} is 4-pyrimidinyl; wherein each pyrimidinyl is optionally substituted with one or two R^{A1}.

In a further preferred embodiment, R^{A} is 4-pyrimidinyl; wherein each pyrimidinyl is unsubstituted.

In a further preferred embodiment, R^{A} is 5-(2-pyridonyl); wherein each pyridonyl is optionally substituted with one or two R^{A1}.

In a further preferred embodiment, R^{A} is 5-(2-pyridonyl); wherein each pyridonyl is unsubstituted.

In some preferred embodiments, R^{A1} is independently halogen, —OH, oxo, —C₁-C₄ alkyl, or —C₃-C₆ cycloalkyl, wherein each alkyl or cycloalkyl is optionally substituted with one or more halogen or —OH. In a further preferred embodiment, R^{A1} is independently halogen, —OH, —C₁-C₄ alkyl, or —C₃-C₆ cycloalkyl, wherein each alkyl or cycloalkyl is optionally substituted with one or more halogen or —OH. In a further preferred embodiment, R^{A1} is independently halogen, —OH, or —C₁-C₄ alkyl, wherein each alkyl is optionally substituted with one or more halogen or —OH. In a further preferred embodiment, R^{A1} is independently halogen or —C₁-C₄ alkyl, wherein each alkyl is optionally substituted with one or more halogen or —OH. In a further preferred embodiment, R^{A1} is independently halogen or —C₁-C₂ alkyl, wherein each alkyl is optionally substituted with one or more halogen or —OH. In a further preferred embodiment, R^{A1} is independently fluorine, chlorine, or —C₁-C₂ alkyl, wherein each alkyl is optionally substituted with one or more fluorine, chlorine, or —OH. In a further preferred embodiment, R^{A1} is independently fluorine, chlorine, or —C₁-C₂ alkyl, wherein each alkyl is optionally substituted with one or more —OH. In a further preferred embodiment, R^{A1} is independently fluorine, chlorine, or —C₁-C₂ alkyl, wherein each alkyl is optionally substituted with one or two, preferably one —OH.

In some preferred embodiments, R^{A} is selected from:

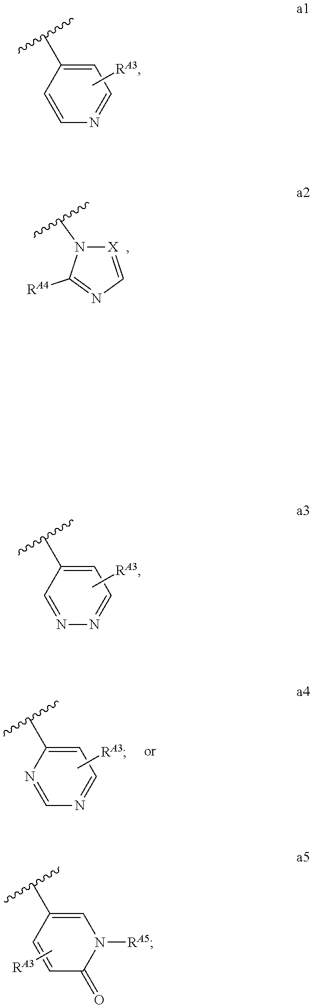

wherein
X is —CH— or N;
R^{A3} is independently, at each occurrence, 1 to 3 identical or different substituents selected from hydrogen, halogen, —C₁-C₈ alkyl, —C₁-C₈ haloalkyl, —C₁-C₈ alkoxy, —C₁-C₈ hydroxyalkyl, and —C₁-C₈ alkoxyalkyl;
R^{A4} is independently, at each occurrence, hydrogen, halogen, —C₁-C₈ alkyl, —C₃-C₈ cycloalkyl, —C₁-C₈ hydroxyalkyl, and —C₁-C₈ alkoxyalkyl; and
R^{A5} is independently, at each occurrence, hydrogen or —C₁-C₈ alkyl.

In some preferred embodiments, R^{A} is selected from:

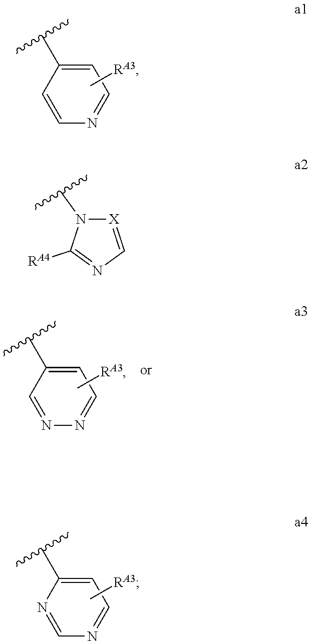

wherein
X is —CH— or N;
R^{A3} is independently, at each occurrence, 1 to 3 identical or different substituents selected from hydrogen, halogen, —C₁-C₈ alkyl, —C₁-C₈ haloalkyl, —C₁-C₈ alkoxy, —C₁-C₈ hydroxyalkyl, and —C₁-C₈ alkoxyalkyl; and
R^{A4} is independently, at each occurrence, hydrogen, halogen, —C₁-C₈ alkyl, —C₃-C₈ cycloalkyl, —C₁-C₈ hydroxyalkyl, and —C₁-C₈ alkoxyalkyl.

In a further preferred embodiment, R^{A3} represents one or two substituents, preferably one substituent.

In a further preferred embodiment, R^{A} is selected from:

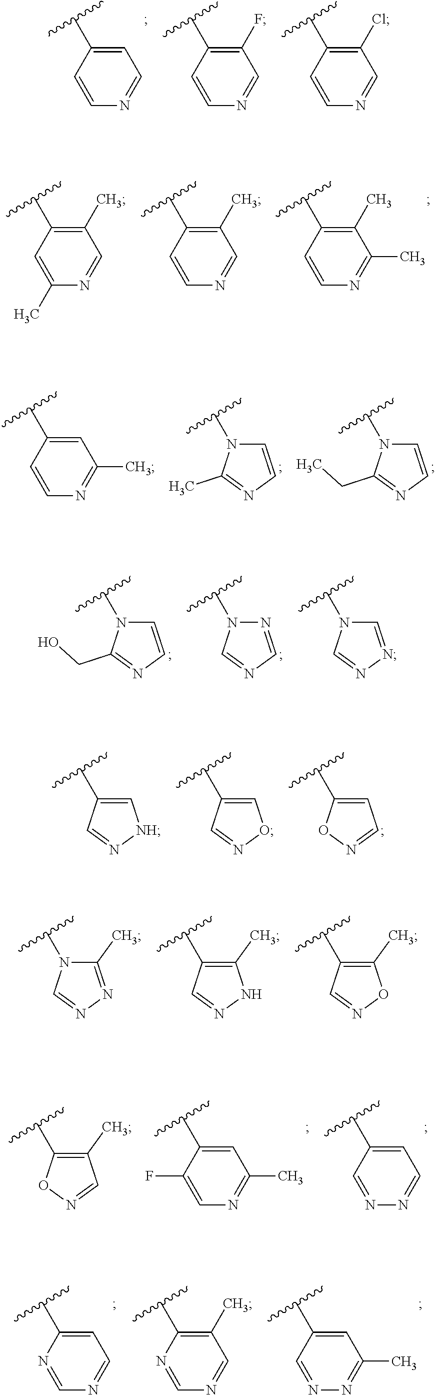

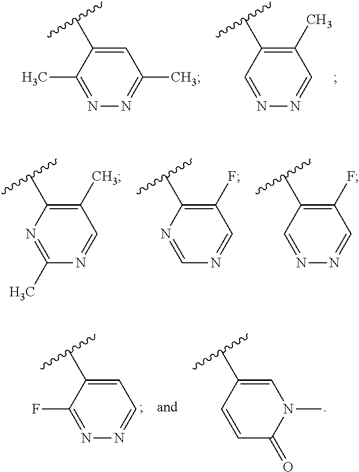

In a further preferred embodiment, R^{A} is selected from:

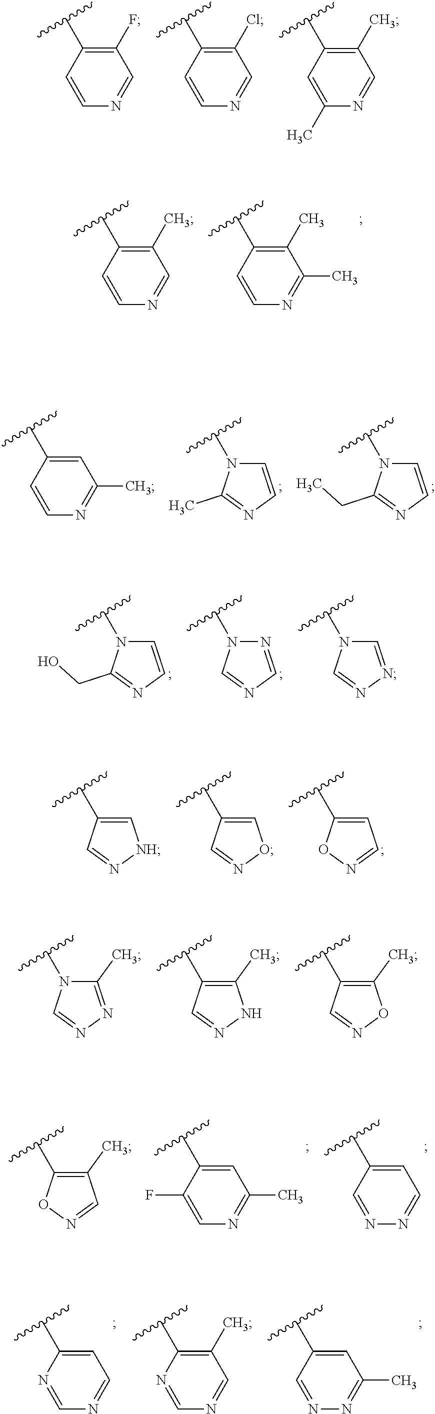

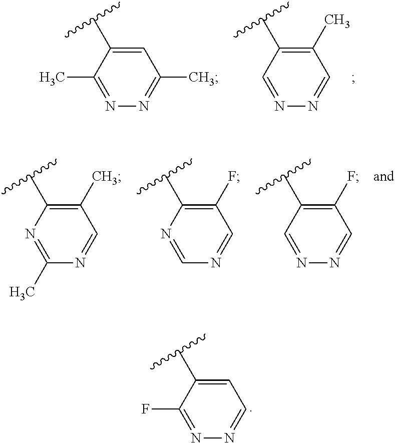

In a further preferred embodiment, R^{A} is selected from:

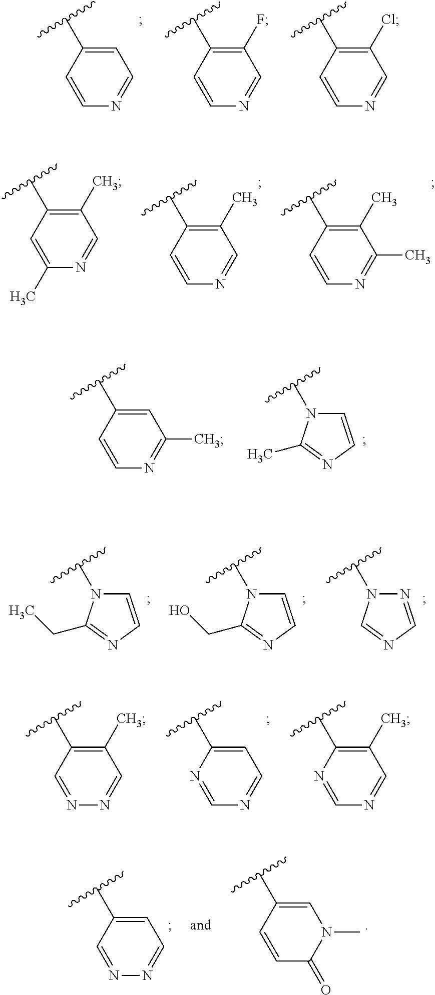

In a further preferred embodiment, R^{A} is selected from:

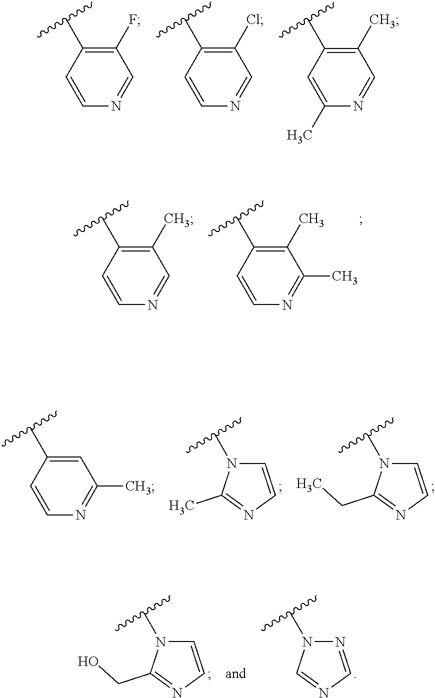

In some preferred embodiments, R^{B} is selected from phenyl, oxazolyl, pyridinyl, pyrimidinyl, pyridazinyl, and pyrazinyl, wherein each phenyl, oxazolyl, pyridinyl, pyrimidinyl, pyridazinyl, and pyrazinyl is optionally substituted by one or more R^{B1}. In a further preferred embodiment, R^{B} is selected from phenyl, 2-oxazolyl, 2-pyridinyl, 3-pyridinyl, 4-pyridinyl, 2-pyrimidinyl, 4-pyrimidinyl, 5-pyrimidinyl, 4-pyridazinyl, and 2-pyrazinyl, wherein each phenyl, oxazolyl, pyridinyl, pyrimidinyl, pyridazinyl, and pyrazinyl is optionally substituted by one or more R^{B1}. In a further preferred embodiment, R^{B} is selected from phenyl, 2-oxazolyl, 2-pyridinyl, 3-pyridinyl, 2-pyrimidinyl, 4-pyrimidinyl, 5-pyrimidinyl, 4-pyridazinyl, and 2-pyrazinyl, wherein each phenyl, oxazolyl, pyridinyl, pyrimidinyl, pyridazinyl, and pyrazinyl is optionally substituted by one or two, preferably one R^{B1}. In a further preferred embodiment, R^{B} is selected from phenyl, oxazolyl, pyridinyl, and pyridinyl, wherein each phenyl, oxazolyl, and pyridinyl is optionally substituted by one or two, preferably one R^{B1}. In a further preferred embodiment, R^{B} is selected from phenyl, 2-oxazolyl, 2-pyridinyl, and 3-pyridinyl, wherein each phenyl, oxazolyl, and pyridinyl is optionally substituted by one or two, preferably one R^{B1}.

In a further preferred embodiment, R^{B} is selected from phenyl, pyridinyl, pyrimidinyl, pyridazinyl, and pyrazinyl, wherein each phenyl, pyridinyl, pyrimidinyl, pyridazinyl, and pyrazinyl is optionally substituted by one or more R^{B1}.

In a further preferred embodiment, R^{B} is selected from phenyl, 2-pyridinyl, 3-pyridinyl, 2-pyrimidinyl, 4-pyrimidinyl, 5-pyrimidinyl, 4-pyridazinyl, and 2-pyrazinyl, wherein each phenyl, pyridinyl, pyrimidinyl, pyridazinyl, and pyrazinyl is optionally substituted by one or more R^{B1}.

In a further preferred embodiment, R^{B} is from phenyl, wherein said phenyl is optionally substituted by one more R^{B1}. In a further preferred embodiment, R^{B} is from phenyl, wherein said phenyl is optionally substituted by one or two, preferably one R^{B1}.

In some preferred embodiments, R^{B1} is selected from halogen, —OH, cyano, —C₁-C₄ alkyl, and —C₁-C₄ alkoxy, wherein each alkyl and alkoxy is independently substituted with one or more halogen, —OH, or —C₁-C₄ alkoxy. In a further preferred embodiment, R^{B1} is selected from halogen, —OH, cyano, and —C₁-C₄ alkyl, wherein said alkyl is independently substituted with one or more halogen, —OH, or —C₁-C₄ alkoxy. In a further preferred embodiment, R^{B1} is selected from halogen, —OH, cyano, and —C₁-C₄ alkyl, wherein said alkyl is independently substituted with one or more halogen or —OH. In a further preferred embodiment, R^{B1} is selected from halogen, —OH, cyano, and —C₁-C₂ alkyl, wherein said alkyl is independently substituted with one or more halogen or —OH. In a further preferred embodiment, R^{B1} is selected from halogen, —OH, and cyano. In a further preferred embodiment, R^{B1} is selected from halogen and cyano. In a further preferred embodiment, R^{B1} is selected from fluorine, chlorine, and cyano. In a further preferred embodiment, R^{B1} is selected from fluorine and cyano. In a further preferred embodiment, R^{B1} is fluorine. In a further preferred embodiment, R^{B1} is cyano.

In some preferred embodiments, R^{B} is selected from:

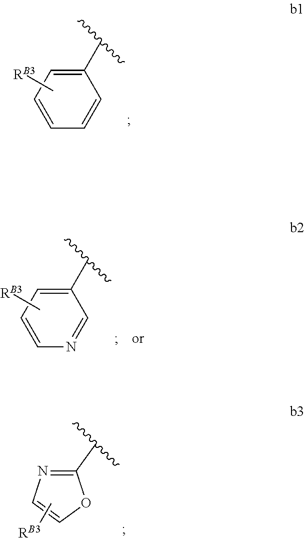

wherein
R^{B3} is independently, at each occurrence, selected from hydrogen, halogen, —C₁-C₈ alkyl, —C₁-C₈ haloalkyl, —C₁-C₈ alkoxy, —C₁-C₈ hydroxyalkyl, —C₁-C₈ alkoxyalkyl, and —CN. In a further preferred embodiment, R^{B3} represents 1 to 3 identical or different substituents for b1 and b2, and 1 to 2 identical or different substituents for b3. In a further preferred embodiment, R^{B3} represents 1 to 2 identical or different substituents, preferably 1 substituent. In a further preferred embodiment, R^{B} is selected from:

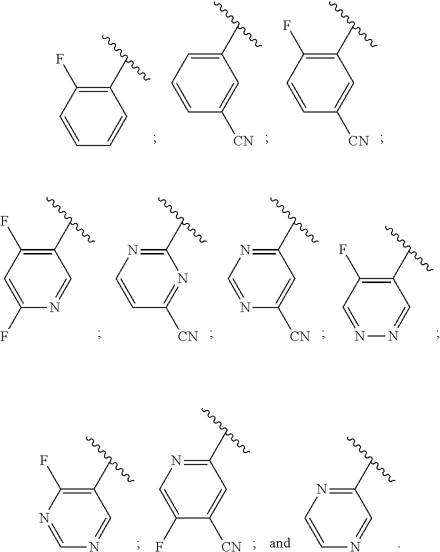

In a further preferred embodiment, R^{B} is selected from

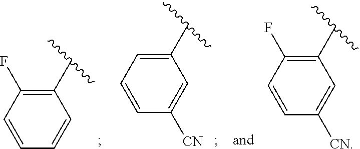 [SMILES: *c1ccccc1F ; *c1cccc(C#N)c1 ; *c1cc(C#N)ccc1F]

In a further preferred embodiment, R^{B} is selected from

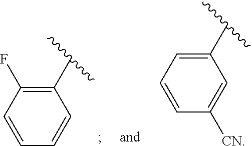 [SMILES: *c1ccccc1F ; *c1cccc(C#N)c1]

In some preferred embodiments, R^{B} is phenyl substituted with one or two R^{B1}, wherein R^{B1} is independently, at each occurrence, cyano or fluoro. In a further preferred embodiment, R^{B} is 2-fluorophenyl, 3-cyanophenyl, or 2-fluoro-5-cyanophenyl. In a further preferred embodiment, R^{B} is 2-fluorophenyl. In a further preferred embodiment, R^{B} is 3-cyanophenyl. In a further preferred embodiment, R^{B} is 2-fluoro-5-cyanophenyl.

In some embodiments, the present invention provides a compound of Formula I:

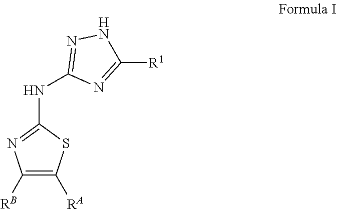

wherein:
R¹ is H, halogen, —C₁-C₂ alkyl, or —C₃-C₄ cycloalkyl, wherein each alkyl or cycloalkyl is optionally substituted with one or more R²;
R² is independently, at each occurrence, selected from halogen, —OH, and —OC₁-C₂ alkyl;
R^{A} is imidazolyl, triazolyl, pyridinyl, pyridazinyl, pyrimidinyl, or pyridonyl; wherein each imidazolyl, triazolyl, pyridinyl, pyridazinyl, pyrimidinyl or pyridonyl is optionally substituted with one or more R^{A1};
R^{A1} is independently halogen, —OH, —C₁-C₄ alkyl, or —C₃-C₆ cycloalkyl, wherein each alkyl or cycloalkyl is optionally substituted with one or more halogen or —OH;
R^{B} is selected from phenyl, pyridinyl, pyrimidinyl, pyridazinyl, and pyrazinyl, wherein each phenyl, pyridinyl, pyrimidinyl, pyridazinyl, and pyrazinyl is optionally substituted by one or more R^{B1}; and
R^{B1} is independently, at each occurrence, selected from halogen, and cyano; or a pharmaceutically acceptable salt, solvate, or hydrate thereof.

In some embodiments, the present invention provides a compound of Formula I:

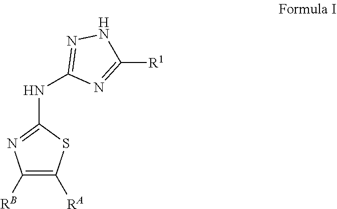

wherein:
R¹ is H, halogen, —C₁-C₈ alkyl, —C₃-C₈ cycloalkyl, —O—C₁-C₈ alkyl, or —CN, wherein each alkyl, cycloalkyl, or —O-alkyl is optionally substituted with one or more R²;
R² is independently, at each occurrence, selected from halogen, —OH, and —OC₁-C₈ alkyl;
R^{A} is a 5- to 6-membered heteroaryl, wherein said heteroaryl is optionally substituted with one or more R^{A1};
R^{A1} is independently, at each occurrence, selected from halogen, —OH, oxo, cyano, —C₁-C₈ alkyl, —C₃-C₈ cycloalkyl, and —O—C₁-C₈ alkyl, wherein each alkyl, cycloalkyl, or —O-alkyl is optionally substituted with one or more R^{A2};
R^{A2} is independently, at each occurrence, halogen, —OC₁-C₈ alkyl, or —OH;
R^{B} is phenyl, wherein said phenyl is optionally substituted with one or more R^{B1}; and
R^{B1} is independently, at each occurrence, selected from halogen, —OH, cyano, —C₁-C₈ alkyl, and —O—C₁-C₈ alkyl, wherein each alkyl and —O-alkyl is optionally substituted with one or more R^{B2}; and
R^{B2} is independently, at each occurrence, halogen, —OH, or —OC₁-C₈ alkyl; or a pharmaceutically acceptable salt, solvate, or hydrate thereof.

In some embodiments, the present invention provides a compound of Formula I:

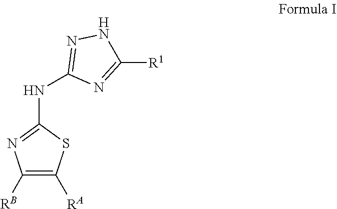

wherein:
R¹ is H, halogen, —C₁-C₂ alkyl, or —C₃-C₄ cycloalkyl, wherein each alkyl or cycloalkyl is optionally substituted with one or more R²;
R² is independently, at each occurrence, selected from halogen, —OH, and —OC₁-C₂ alkyl;
R^{A} is imidazolyl, triazolyl, pyridinyl, pyridazinyl, pyrimidinyl, or pyridonyl; wherein each imidazolyl, triazolyl, pyridinyl, pyridazinyl, pyrimidinyl or pyridonyl is optionally substituted with one or more R^{A1};
R^{A1} is independently halogen, —OH, —C₁-C₄ alkyl, or —C₃-C₆ cycloalkyl, wherein each alkyl or cycloalkyl is optionally substituted with one or more halogen or —OH;
R^{B} is phenyl, wherein said phenyl is optionally substituted by one or more R^{B1}; and
R^{B1} is independently, at each occurrence, selected from halogen, and cyano;
   or a pharmaceutically acceptable salt, solvate, or hydrate thereof.

In some embodiments, the present invention provides a compound of Formula I:

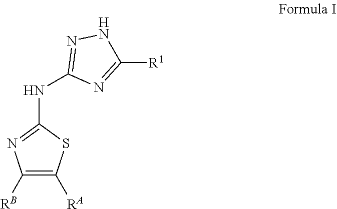

wherein:
R¹ is H, —C₁-C₂ alkyl, or —C₃-C₄ cycloalkyl, wherein each alkyl is optionally substituted with one R²;
R² is independently, at each occurrence, selected from fluoro, chloro or —OH, wherein preferably R² is OH;
R^{A} is 1-imidazolyl, 1-(1,2,4)-triazolyl, 4-pyridinyl, 4-pyridazinyl, 4-pyrimidinyl, or 5-(2-pyridonyl); wherein each imidazolyl, triazolyl, pyridinyl, pyridazinyl, pyrimidinyl or pyridonyl is optionally substituted with one or more R^{A1};
R^{A1} is independently fluorine, chlorine, or —C₁-C₂ alkyl, wherein each alkyl is optionally substituted with one or more fluorine, chlorine, or —OH, preferably wherein each alkyl is optionally substituted with one or more —OH;
R^{B} is phenyl, wherein said phenyl is optionally substituted by one or more R^{B1}; and
R^{B1} is independently, at each occurrence, selected from halogen, and cyano;
   or a pharmaceutically acceptable salt, solvate, or hydrate thereof.

In some preferred embodiments, the compound is of the Formula Ia1b:

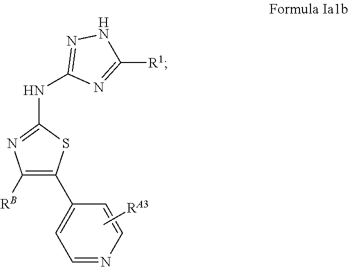

wherein R¹, R^{B}, R^{A3}, and R^{A4} are as defined in any of the embodiments above.

In some preferred embodiments, the compound is of the Formula Ia2b:

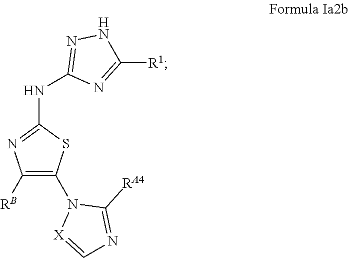

wherein R¹, R^{B}, R^{A4} and X are as defined in any of the embodiments above.

In some preferred embodiments, the compound is of the Formula Ia3b:

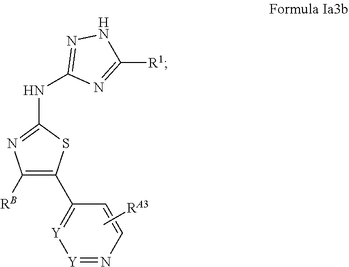

wherein R¹, R^{B}, and R^{A3} are as defined in any of the embodiments above, and Y is independently, at each occurrence, —CH— or N, wherein at least one of Y is —CH—.

In some preferred embodiments, the compound is of the Formula Ia1b or Ia2b:

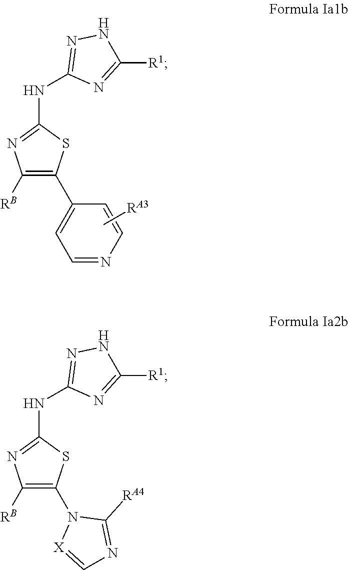

wherein R¹, R^{B}, R^{A3}, R^{A4} and X are as defined in any of the embodiments above; preferably wherein:
R¹ is H, halogen, —C₁-C₈ alkyl, —C₃-C₈ cycloalkyl, —O—C₁-C₈ alkyl, or —CN, wherein each alkyl, cycloalkyl, or —O-alkyl is optionally substituted with one or more R²;
R² is independently, at each occurrence, selected from halogen, —OH, and —OC₁-C₈ alkyl;
X is —CH— or N;
R^{A3} is independently, at each occurrence, 1 to 3 identical or different substituents selected from hydrogen, halogen, —C₁-C₈ alkyl, —C₁-C₈ haloalkyl, —C₁-C₈ alkoxy, —C₁-C₈ hydroxyalkyl, and —C₁-C₈ alkoxyalkyl;
R^{A4} is independently, at each occurrence, hydrogen, halogen, —C₁-C₈ alkyl, —C₃-C₈ cycloalkyl, —C₁-C₈ hydroxyalkyl, and —C₁-C₈ alkoxyalkyl;
R^{B} is phenyl or a 5- to 6-membered heteroaryl, wherein each phenyl or heteroaryl is optionally substituted with one or more R^{B1};
R^{B1} is independently, at each occurrence, selected from halogen, —OH, cyano, —C₁-C₈ alkyl, and —O—C₁-C₈ alkyl, wherein each alkyl and —O-alkyl is optionally substituted with one or more R^{B2}; and
   R^{B2} is independently, at each occurrence, halogen, —OH, or —OC₁-C₈ alkyl.

In some preferred embodiments, the compound is of the Formula Ia3b or Ia2b:

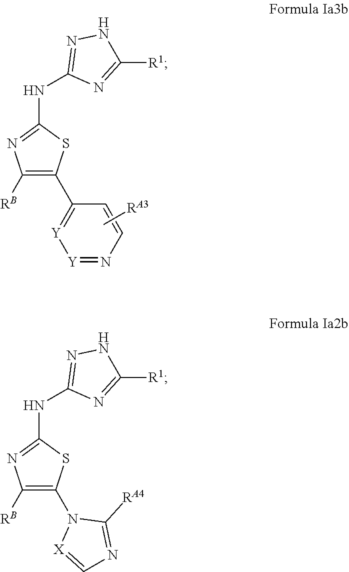

wherein R¹, R^{B}, R^{A3}, R^{A4} X and Y are as defined in any of the embodiments above; preferably wherein:
R¹ is H, halogen, —C₁-C₈ alkyl, —C₃-C₈ cycloalkyl, —O—C₁-C₈ alkyl, or —CN, wherein each alkyl, cycloalkyl, or —O-alkyl is optionally substituted with one or more R²;
R² is independently, at each occurrence, selected from halogen, —OH, and —OC₁-C₈ alkyl;
X is —CH— or N;
Y is independently, at each occurrence, —CH— or N, wherein at least one of Y is —CH—;
R^{A3} is independently, at each occurrence, 1 to 3 identical or different substituents selected from hydrogen, halogen, —C₁-C₈ alkyl, —C₁-C₈ haloalkyl, —C₁-C₈ alkoxy, —C₁-C₈ hydroxyalkyl, and —C₁-C₈ alkoxyalkyl;
R^{A4} is independently, at each occurrence, hydrogen, halogen, —C₁-C₈ alkyl, —C₃-C₈ cycloalkyl, —C₁-C₈ hydroxyalkyl, and —C₁-C₈ alkoxyalkyl;
R^{B} is phenyl or a 5- to 6-membered heteroaryl, wherein each phenyl or heteroaryl is optionally substituted with one or more R^{B1};
R^{B1} is independently, at each occurrence, selected from halogen, —OH, cyano, —C₁-C₈ alkyl, and —O—C₁-C₈ alkyl, wherein each alkyl and —O-alkyl is optionally substituted with one or more R^{B2}; and
R^{B2} is independently, at each occurrence, halogen, —OH, or —OC₁-C₈ alkyl.

In some preferred embodiments, the compound is of the Formula Ia1b1, Ia1b2, Ia1b3, Ia2b1, Ia2b2, Ia2b3, Ia3b1, Ia3b2, or Ia3b3:

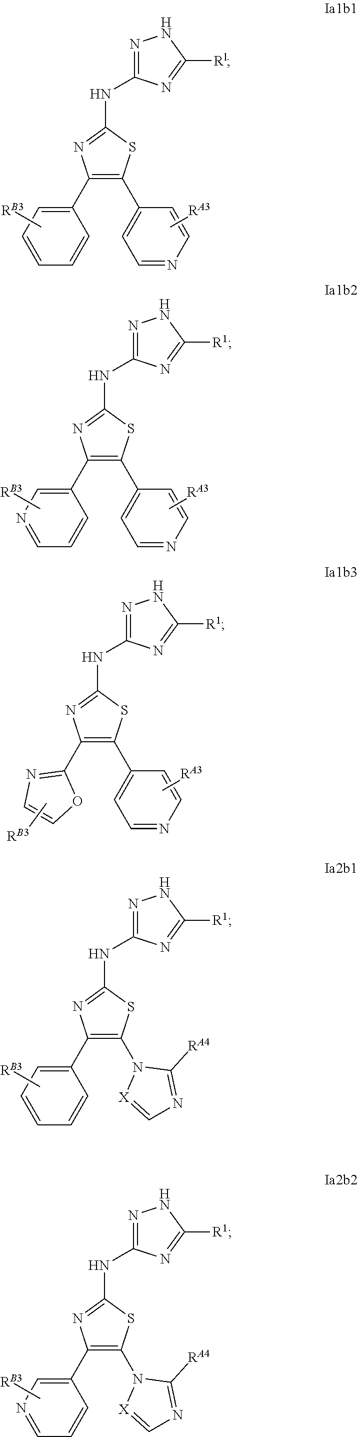

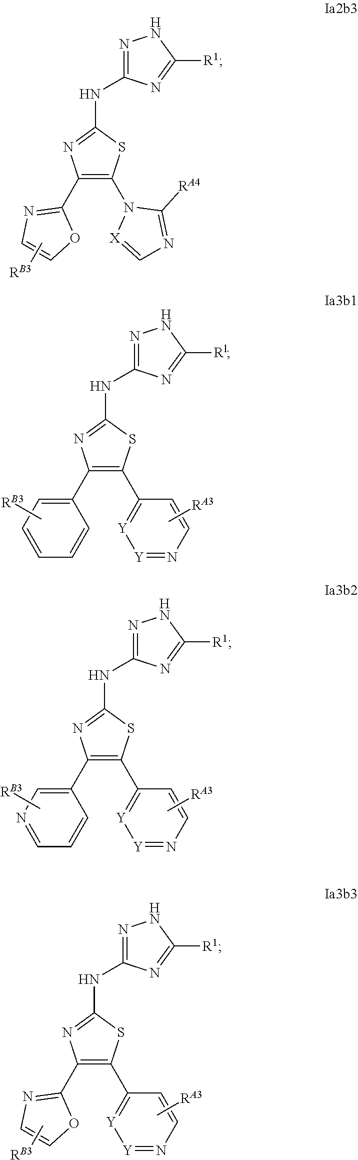

wherein R¹, R^{B3}, R^{A3}, R^{A4} and X are as defined in any of the embodiments above; preferably wherein:
R¹ is H, halogen, —C₁-C₈ alkyl, —C₃-C₈ cycloalkyl, —O—C₁-C₈ alkyl, or —CN, wherein each alkyl, cycloalkyl, or —O-alkyl is optionally substituted with one or more R²;
R² is independently, at each occurrence, selected from halogen, —OH, and —OC₁-C₈ alkyl;
X is —CH— or N;
Y is independently, at each occurrence, —CH— or N, wherein at least one of Y is —CH—;
R^{A3} is independently, at each occurrence, 1 to 3 identical or different substituents selected from hydrogen, halogen, —C₁-C₈ alkyl, —C₁-C₈ haloalkyl, —C₁-C₈ alkoxy, —C₁-C₈ hydroxyalkyl, and —C₁-C₈ alkoxyalkyl;
R^{A4} is independently, at each occurrence, hydrogen, halogen, —C₁-C₈ alkyl, —C₃-C₈ cycloalkyl, —C₁-C₈ hydroxyalkyl, and —C₁-C₈ alkoxyalkyl; and
R^{B3} is independently, at each occurrence, selected from hydrogen, halogen, —C₁-C₈ alkyl, —C₁-C₈ haloalkyl, —C₁-C₈ alkoxy, —C₁-C₈ hydroxyalkyl, —C₁-C₈ alkoxyalkyl, and —CN.

In a further preferred embodiment, the compound is a compound of Formula Ia1b1 wherein R^{B3} is one R^{B3} selected from cyano and fluoro; and R¹ is selected from —C₃-C₄ cycloalkyl. In some preferred embodiments, the compound is a compound of Formula Ia2b1 where R^{B3} is one R^{B3} selected from cyano or fluoro; and R¹ is selected from —C₃-C₄ cycloalkyl. In some preferred embodiments, the compound is a compound of Formula Ia1b1 wherein R^{B3} is 3-cyano; and R¹ is —C₃-C₄ cycloalkyl. In some preferred embodiments, the compound is a compound of Formula 1a1b1 where R^{B3} is 2-fluoro; and R¹ is —C₃-C₄ cycloalkyl. In some preferred embodiments, the compound is a compound of Formula Ia2b1 where R^{B3} is 2-fluoro and R¹ is —C₃-C₄ cycloalkyl. In some preferred embodiments, the compound is a compound of Formula Ia2b1 wherein R^{B3} is 3-cyano and R¹ is —C₃-C₄ cycloalkyl.

In a preferred embodiment, R^{A} is pyridine optionally substituted with one or two R^{A1}, wherein:
R^{A1} is independently, at each occurrence, fluoro, chloro, or methyl; and
R^{B} is 3-cyanophenyl.

In a preferred embodiment, R^{A} is 1-imidazole optionally substituted with one or two R^{A1}, wherein:
R^{A1} is independently, at each occurrence, methyl, ethyl, or hydroxymethyl; and
R^{B} is 3-cyanophenyl.

In a preferred embodiment, R¹ is H; R^{A} is 1-imidazole optionally substituted with one or two R^{A1}, wherein:
R^{A1} is independently, at each occurrence, methyl, ethyl, or hydroxymethyl; and
R^{B} is 3-cyanophenyl.

In a preferred embodiment, R^{A} is 1-imidazole optionally substituted once at the 2-position with R^{A1}, wherein:
R^{A1} is independently, at each occurrence, methyl, ethyl, or hydroxymethyl; and
R^{B} is 3-cyanophenyl.

In a preferred embodiment, R^{A} is 1-imidazole optionally substituted once at the 2-position with R^{A1}, wherein:
R^{A1} is methyl; and
R^{B} is 3-cyanophenyl.

In a preferred embodiment, R^{A} is triazole; and

R^{B} is 3-cyanophenyl.

In some preferred embodiments, said compound is selected from:
5-cyclobutyl-N-[4-(2-fluorophenyl)-5-(3-fluoropyridin-4-yl)-1,3-thiazol-2-yl]-1H-1,2,4-triazol-3-amine;
3-[5-(3-chloropyridin-4-yl)-2-[(5-cyclopropyl-1H-1,2,4-triazol-3-yl)amino]-1,3-thiazol-4-yl]benzonitrile;
3-{2-[(5-cyclopropyl-1H-1,2,4-triazol-3-yl)amino]-5-(3-fluoropyridin-4-yl)-1,3-thiazol-4-yl}benzonitrile;
3-{2-[(5-cyclopropyl-1H-1,2,4-triazol-3-yl)amino]-5-(2,5-dimethylpyridin-4-yl)-1,3-thiazol-4-yl}benzonitrile;
3-{2-[(5-cyclopropyl-1H-1,2,4-triazol-3-yl)amino]-5-(3-methylpyridin-4-yl)-1,3-thiazol-4-yl}benzonitrile;
3-{2-[(5-cyclopropyl-1H-1,2,4-triazol-3-yl)amino]-5-(2,3-dimethylpyridin-4-yl)-1,3-thiazol-4-yl}benzonitrile;
3-{2-[(5-cyclopropyl-1H-1,2,4-triazol-3-yl)amino]-5-(2-methyl-1H-imidazol-1-yl)-1,3-thiazol-4-yl}benzonitrile;
3-{2-[(5-cyclopropyl-1H-1,2,4-triazol-3-yl)amino]-5-(2-methylpyridin-4-yl)-1,3-thiazol-4-yl}benzonitrile;
3-[5-(2-methyl-1H-imidazol-1-yl)-2-[(1H-1,2,4-triazol-3-yl)amino]-1,3-thiazol-4-yl]benzonitrile;
3-{2-[(5-methyl-1H-1,2,4-triazol-3-yl)amino]-5-(2-methyl-1H-imidazol-1-yl)-1,3-thiazol-4-yl}benzonitrile;
3-(2-{[5-(hydroxymethyl)-1H-1,2,4-triazol-3-yl]amino}-5-(2-methyl-1H-imidazol-1-yl)-1,3-thiazol-4-yl)benzonitrile;
3-[5-(2,5-dimethylpyridin-4-yl)-2-[(1H-1,2,4-triazol-3-yl)amino]-1,3-thiazol-4-yl]benzonitrile;
3-[5-(2,5-dimethylpyridin-4-yl)-2-[(5-methyl-1H-1,2,4-triazol-3-yl)amino]-1,3-thiazol-4-yl]benzonitrile;
3-[5-(2,5-dimethylpyridin-4-yl)-2-{[5-(hydroxymethyl)-1H-1,2,4-triazol-3-yl]amino}-1,3-thiazol-4-yl]benzonitrile;
3-{2-[(5-cyclopropyl-1H-1,2,4-triazol-3-yl)amino]-5-(1H-1,2,4-triazol-1-yl)-1,3-thiazol-4-yl}benzonitrile;
3-[5-(2-ethyl-1H-imidazol-1-yl)-2-[(5-methyl-1H-1,2,4-triazol-3-yl)amino]-1,3-thiazol-4-yl]benzonitrile;
3-{2-[(5-cyclopropyl-1H-1,2,4-triazol-3-yl)amino]-5-[2-(hydroxymethyl)-1H-imidazol-1-yl]-1,3-thiazol-4-yl}benzonitrile;
3-{2-[(5-cyclopropyl-1H-1,2,4-triazol-3-yl)amino]-5-(2-methyl-1H-imidazol-1-yl)-1,3-thiazol-4-yl}-4-fluorobenzonitrile;
3-{5-[2-(hydroxymethyl)-1H-imidazol-1-yl]-2-[(1H-1,2,4-triazol-3-yl)amino]-1,3-thiazol-4-yl}benzonitrile;
3-{2-[(5-ethyl-1H-1,2,4-triazol-3-yl)amino]-5-[2-(hydroxymethyl)-1H-imidazol-1-yl]-1,3-thiazol-4-yl}benzonitrile;
3-{5-[2-(hydroxymethyl)-1H-imidazol-1-yl]-2-[(5-methyl-1H-1,2,4-triazol-3-yl)amino]-1,3-thiazol-4-yl}benzonitrile;
3-[5-(3-methylpyridin-4-yl)-2-[(1H-1,2,4-triazol-3-yl)amino]-1,3-thiazol-4-yl]benzonitrile;
3-{2-[(5-cyclopropyl-1H-1,2,4-triazol-3-yl)amino]-5-(5-methylpyridazin-4-yl)-1,3-thiazol-4-yl}benzonitrile;
3-{2-[(5-cyclopropyl-1H-1,2,4-triazol-3-yl)amino]-5-(pyrimidin-4-yl)-1,3-thiazol-4-yl}benzonitrile;
3-{2-[(5-cyclopropyl-1H-1,2,4-triazol-3-yl)amino]-5-(pyridazin-4-yl)-1,3-thiazol-4-yl}benzonitrile;
3-{2-[(5-cyclopropyl-1H-1,2,4-triazol-3-yl)amino]-5-(pyridin-4-yl)-1,3-thiazol-4-yl}benzonitrile;
3-{2-[(5-cyclopropyl-1H-1,2,4-triazol-3-yl)amino]-5-(5-methylpyrimidin-4-yl)-1,3-thiazol-4-yl}benzonitrile;
m-[2-(5-cyclopropyl-1H-1,2,4-triazol-3-ylamino)-5-(2-ethyl-1-imidazolyl)-1,3-thiazol-4-yl]benzonitrile; and
3-{2-[(5-cyclopropyl-1H-1,2,4-triazol-3-yl)amino]-5-(1-methyl-6-oxo-1,6-dihydropyridin-3-yl)-1,3-thiazol-4-yl}benzonitrile.

In a preferred embodiment, said compound is 5-cyclobutyl-N-[4-(2-fluorophenyl)-5-(3-fluoropyridin-4-yl)-1,3-thiazol-2-yl]-1H-1,2,4-triazol-3-amine.

In some preferred embodiment, said compound is 3-[5-(3-chloropyridin-4-yl)-2-[(5-cyclopropyl-1H-1,2,4-triazol-3-yl)amino]-1,3-thiazol-4-yl]benzonitrile.

In some preferred embodiment, said compound is 3-{2-[(5-cyclopropyl-1H-1,2,4-triazol-3-yl)amino]-5-(3-fluoropyridin-4-yl)-1,3-thiazol-4-yl}benzonitrile.

In a preferred embodiment, said compound is 3-{2-[(5-cyclopropyl-1H-1,2,4-triazol-3-yl)amino]-5-(2,5-dimethylpyridin-4-yl)-1,3-thiazol-4-yl}benzonitrile.

In a preferred embodiment, said compound is 3-{2-[(5-cyclopropyl-1H-1,2,4-triazol-3-yl)amino]-5-(3-methylpyridin-4-yl)-1,3-thiazol-4-yl}benzonitrile.

In a preferred embodiment, said compound is 3-{2-[(5-cyclopropyl-1H-1,2,4-triazol-3-yl)amino]-5-(2,3-dimethylpyridin-4-yl)-1,3-thiazol-4-yl}benzonitrile.

In a preferred embodiment, said compound is 3-{2-[(5-cyclopropyl-1H-1,2,4-triazol-3-yl)amino]-5-(2-methyl-1H-imidazol-1-yl)-1,3-thiazol-4-yl}benzonitrile.

In a preferred embodiment, said compound is 3-{2-[(5-cyclopropyl-1H-1,2,4-triazol-3-yl)amino]-5-(2-methylpyridin-4-yl)-1,3-thiazol-4-yl}benzonitrile.

In a preferred embodiment, said compound is 3-[5-(2-methyl-1H-imidazol-1-yl)-2-[(1H-1,2,4-triazol-3-yl)amino]-1,3-thiazol-4-yl]benzonitrile.

In a preferred embodiment, said compound is 3-{2-[(5-methyl-1H-1,2,4-triazol-3-yl)amino]-5-(2-methyl-1H-imidazol-1-yl)-1,3-thiazol-4-yl}benzonitrile.

In a preferred embodiment, said compound is 3-(2-{[5-(hydroxymethyl)-1H-1,2,4-triazol-3-yl]amino}-5-(2-methyl-1H-imidazol-1-yl)-1,3-thiazol-4-yl)benzonitrile.

In a preferred embodiment, said compound is 3-[5-(2,5-dimethylpyridin-4-yl)-2-[(1H-1,2,4-triazol-3-yl)amino]-1,3-thiazol-4-yl]benzonitrile.

In a preferred embodiment, said compound is 3-[5-(2,5-dimethylpyridin-4-yl)-2-[(5-methyl-1H-1,2,4-triazol-3-yl)amino]-1,3-thiazol-4-yl]benzonitrile.

In a preferred embodiment, said compound is 3-[5-(2,5-dimethylpyridin-4-yl)-2-{[5-(hydroxymethyl)-1H-1,2,4-triazol-3-yl]amino}-1,3-thiazol-4-yl]benzonitrile.

In a preferred embodiment, said compound is 3-{2-[(5-cyclopropyl-1H-1,2,4-triazol-3-yl)amino]-5-(1H-1,2,4-triazol-1-yl)-1,3-thiazol-4-yl}benzonitrile.

In a preferred embodiment, said compound is 3-[5-(2-ethyl-1H-imidazol-1-yl)-2-[(5-methyl-1H-1,2,4-triazol-3-yl)amino]-1,3-thiazol-4-yl]benzonitrile.

In a preferred embodiment, said compound is 3-{2-[(5-cyclopropyl-1H-1,2,4-triazol-3-yl)amino]-5-[2-(hydroxymethyl)-1H-imidazol-1-yl]-1,3-thiazol-4-yl}benzonitrile.

In a preferred embodiment, said compound is 3-{2-[(5-cyclopropyl-1H-1,2,4-triazol-3-yl)amino]-5-(2-methyl-1H-imidazol-1-yl)-1,3-thiazol-4-yl}-4-fluorobenzonitrile.

In a preferred embodiment, said compound is 3-{5-[2-(hydroxymethyl)-1H-imidazol-1-yl]-2-[(1H-1,2,4-triazol-3-yl)amino]-1,3-thiazol-4-yl}benzonitrile.

In a preferred embodiment, said compound is 3-{2-[(5-ethyl-1H-1,2,4-triazol-3-yl)amino]-5-[2-(hydroxymethyl)-1H-imidazol-1-yl]-1,3-thiazol-4-yl}benzonitrile.

In a preferred embodiment, said compound is 3-{5-[2-(hydroxymethyl)-1H-imidazol-1-yl]-2-[(5-methyl-1H-1,2,4-triazol-3-yl)amino]-1,3-thiazol-4-yl}benzonitrile.

In a preferred embodiment, said compound is 3-[5-(3-methylpyridin-4-yl)-2-[(1H-1,2,4-triazol-3-yl)amino]-1,3-thiazol-4-yl]benzonitrile.

In a preferred embodiment, said compound is 3-{2-[(5-cyclopropyl-1H-1,2,4-triazol-3-yl)amino]-5-(5-methylpyridazin-4-yl)-1,3-thiazol-4-yl}benzonitrile.

In a preferred embodiment, said compound is 3-{2-[(5-cyclopropyl-1H-1,2,4-triazol-3-yl)amino]-5-(pyrimidin-4-yl)-1,3-thiazol-4-yl}benzonitrile.

In a preferred embodiment, said compound is 3-{2-[(5-cyclopropyl-1H-1,2,4-triazol-3-yl)amino]-5-(pyridazin-4-yl)-1,3-thiazol-4-yl}benzonitrile.

In a preferred embodiment, said compound is 3-{2-[(5-cyclopropyl-1H-1,2,4-triazol-3-yl)amino]-5-(pyridin-4-yl)-1,3-thiazol-4-yl}benzonitrile.

In a preferred embodiment, said compound is 3-{2-[(5-cyclopropyl-1H-1,2,4-triazol-3-yl)amino]-5-(5-methylpyrimidin-4-yl)-1,3-thiazol-4-yl}benzonitrile.

In a preferred embodiment, said compound is m-[2-(5-cyclopropyl-1H-1,2,4-triazol-3-ylamino)-5-(2-ethyl-1-imidazolyl)-1,3-thiazol-4-yl]benzonitrile.

In a preferred embodiment, said compound is 3-{2-[(5-cyclopropyl-1H-1,2,4-triazol-3-yl)amino]-5-(1-methyl-6-oxo-1,6-dihydropyridin-3-yl)-1,3-thiazol-4-yl}benzonitrile.

Many of the inventive compounds of Formula I are capable of forming acid addition salts, particularly pharmaceutically acceptable acid addition salts. Pharmaceutically acceptable acid addition salts of the compound of Formula I include those of inorganic acids, for example, hydrohalic acids such as hydrofluoric acid, hydrochloric acid, hydrobromic acid or hydroiodic acid, nitric acid, sulfuric acid, phosphoric acid; and organic acids, for example aliphatic monocarboxylic acids such as formic acid, acetic acid, trifluoroacetic acid, propionic acid and butyric acid, aliphatic hydroxy acids such as lactic acid, citric acid, tartaric acid or malic acid, dicarboxylic acids such as maleic acid or succinic acid, aromatic carboxylic acids such as benzoic acid, p-chlorobenzoic acid, diphenylacetic acid or triphenylacetic acid, aromatic hydroxy acids such as o-hydroxybenzoic acid, phydroxybenzoic acid, 1-hydroxynaphthalene-2-carboxylic acid or 3-hydroxynaphthalene-2carboxylic acid, and sulfonic acids such as methanesulfonic acid or benzenesulfonic acid. These salts may be prepared from compounds of Formula I by known salt-forming procedures.

A “hydrate” refers to an association or complex of one or more water molecules and a compound of the invention. The hydrates can be stoichiometric or non-stoichiometric. Particularly preferred examples of hydrates include hemihydrates, monohydrates and dihydrates.

### Methods of Treatment

As shown below in Examples 30-32 and Tables 1 and 2, the inventive compounds disclosed herein selectively inhibit the adenosine A₁ and A_{2B}, specifically the A_{2B} receptor. In particular, the inventive compounds selectively inhibit the A_{2B} and/or A₁ receptors over the A₃ and/or A_{2A} receptors. Accordingly, in preferred embodiments the inventive compounds are used for treating a condition, disease or disorder mediated by activation of the adenosine A_{2B} receptor.

For instance, Examples 30 and 31 and Table 1 demonstrate that compounds of the present invention selectively inhibit A_{2B} and A₁ receptors with EC₅₀ values generally in the low nanomolar range. In contrast, Table 1 shows that compounds of the invention are largely inactive against human A₃ and A_{2A} receptors.

Moreover, Example 32 teaches that compounds of the invention selectively bind to human A_{2B} receptors compared to A_{2A} receptors. As shown in Example 32 and Table 2, compounds of the present invention can selectively stabilize human A_{2B} receptor, leading to an increased melting point and a higher ΔTₘ. In contrast, compounds of the invention have a much smaller impact on ΔTₘ in human A_{2A} receptors, indicating weaker binding with the A_{2A} receptor.

Due to their ability to inhibit adenosine A₁ and/or A_{2B} receptor activation, compounds of Formula I and pharmaceutically acceptable salts or hydrates thereof in accordance with the present invention are useful in the treatment or prevention of conditions, disorders and diseases which are mediated by the activation of the adenosine A₁ and/or A_{2B} receptor. Said conditions, disorders and diseases are particularly selected from a respiratory disease, an inflammatory obstructive airway disease, an inflammatory disease, a metabolic disease, a renal disease, a vascular disease, an allergic disease, an inflammatory gastrointestinal tract disorder, an autoimmune disease, a neurological disorder, an otological disorder, and a cancer. In some embodiments, the compounds and pharmaceutical compositions of the present invention are useful in the treatment of cancer, and hereby in particular ovarian, lung, liver, oral, colon, skin and prostate cancer including melanoma and squamous cell carcinoma. In some embodiments, the compounds and pharmaceutical compositions of the present invention are useful in the treatment of an otological disorder, preferably hearing loss.

In one aspect, the present invention provides a pharmaceutical composition comprising the inventive compound of Formula I, optionally together with a pharmaceutically acceptable diluent or carrier.

Thus, in one aspect, the present invention provides the inventive compound of Formula I or the inventive pharmaceutical composition for use in a method of treating a condition, disorder or disease mediated by activation of the adenosine A₁ and/or A_{2B} receptor.

In one aspect, the present invention provides the use of a compound of Formula I according to the present invention in the manufacture of a medicament for the treatment of a condition, disorder or disease mediated by activation of the adenosine A₁ and/or A_{2B} receptor.

In one aspect, the present invention provides a method of treating a condition, disorder or disease mediated by activation of the adenosine A₁ and/or A_{2B} receptor in a subject in need thereof, comprising administering to the subject a therapeutically effective amount of a compound of Formula I according to the present invention.

In a further aspect, the present invention provides the inventive compound of Formula I or the inventive pharmaceutical composition for use in a method of treating a condition, disorder or disease mediated by activation of the adenosine A_{2B} receptor.

In a further aspect, the present invention provides the inventive compound of Formula I or the inventive pharmaceutical composition for use in a method of treating a condition, disorder or disease ameliorated by the inhibition of the adenosine A_{2B} receptor.

In a further aspect, the present invention provides the inventive compound of Formula I or the inventive pharmaceutical composition for use in a method of treating a condition or disease susceptible to amelioration by antagonism of the adenosine A_{2B} receptor.

In another aspect, the present invention provides the use of a compound of Formula I according to the present invention in the manufacture of a medicament for the treatment of a condition, disorder or disease mediated by activation of the adenosine A_{2B} receptor.

In another aspect, the present invention provides a method of treating a condition, disorder or disease mediated by activation of the adenosine A_{2B} receptor in a subject in need thereof, comprising administering to the subject a therapeutically effective amount of a compound of Formula I according to the present invention.

In another aspect, the present invention provides the inventive compound of Formula I or the inventive pharmaceutical composition for use in a method of treating a condition, disorder or disease selected from a respiratory disease, an inflammatory obstructive airway disease, an inflammatory disease, a metabolic disease, a renal disease, a vascular disease, an allergic disease, an inflammatory gastrointestinal tract disorder, an autoimmune disease, a neurological disorder, an otological disorder, and a cancer.

In another aspect, the present invention provides the use of a compound of Formula I according to the present invention in the manufacture of a medicament for the treatment of a condition, disorder or disease selected from a respiratory disease, an inflammatory obstructive airway disease, an inflammatory disease, a metabolic disease, a renal disease, a vascular disease, an allergic disease, an inflammatory gastrointestinal tract disorder, an autoimmune disease, a neurological disorder, an otological disorder, and a cancer.

In another aspect, the present invention provides a method of treating a condition, disorder or disease selected from a respiratory disease, an inflammatory obstructive airway disease, an inflammatory disease, a metabolic disease, a renal disease, a vascular disease, an allergic disease, an inflammatory gastrointestinal tract disorder, an autoimmune disease, a neurological disorder, an otological disorder and a cancer in a subject in need thereof, wherein said method comprises administering to the subject, particularly a human subject, a therapeutically effective amount of an inventive compound of Formula I or a pharmaceutically acceptable salt, or hydrate thereof.

In a very preferred embodiment, said respiratory disease, inflammatory obstructive airway disease, inflammatory disease, metabolic disease, renal disease, vascular disease, allergic disease, inflammatory gastrointestinal tract disorder, autoimmune disease, neurological disorder, otological disorder or said cancer is selected from pulmonary fibrosis, pulmonary hypertension (PH), chronic obstructive pulmonary disease (COPD), asthma, acute lung injury (ALI), adult respiratory distress syndrome (ARDS), bronchitis, pneumoconiosis, psoriasis, contact dermatitis, atopic dermatitis, conjunctivitis, allergic rhinitis, bowel disease, multiple sclerosis, diabetes, juvenile diabetes, diabetes mellitus, diabetic nephropathy, renal fibrosis, chronic kidney disease (CKD), renal ischemia, hypertension, retinopathy, Parkinson disease, Alzheimer disease, Huntington disease, attention deficit disorder (ADD), attention deficit hyperactivity disorder (ADHD), hearing loss, ovarian cancer, lung cancer, liver cancer, renal cancer, rectal cancer, oral cancer, breast cancer, bladder cancer, colon cancer, skin cancer and prostate cancer including melanoma and squamous cell carcinoma.

Thus, the compounds and pharmaceutical compositions of the present invention are useful in the treatment of inflammatory or obstructive airway diseases, resulting, for example, in reduction of bronchial hyperreactivity, remodeling or disease progression. Inflammatory or obstructive airway diseases for which the present inventive compounds and pharmaceutical compositions are applicable include asthma of any type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Prophylactic efficacy in the treatment of asthma will be evidenced by reduced frequency or severity of symptomatic attack, improvement in lung function or by reduced requirement for other, symptomatic therapy, such anti-inflammatory (e.g. corticosteroid) or bronchodilatory therapy. Further inflammatory or obstructive airway diseases and conditions for which the present inventive compounds and pharmaceutical compositions are useful include acute lung injury (ALI), adult respiratory distress syndrome (ARDS), chronic obstructive pulmonary, airways or lung disease (COPD, COAD or COLD) as well as bronchitis and pneumoconiosis.

Moreover, the compounds and pharmaceutical compositions of the present invention are useful in the treatment of inflammatory or allergic conditions of the skin, for example psoriasis, contact dermatitis and atopic dermatitis, as well as in the treatment of inflammatory diseases or conditions of the eye such as conjunctivitis, or diseases affecting the nose including allergic rhinitis.

Furthermore, the compounds and pharmaceutical compositions of the present invention are useful in the treatment of an inflammatory disease in which autoimmune reactions are implicated or having an autoimmune component, including autoimmune inflammatory bowel disease, multiple sclerosis, diabetes and juvenile diabetes (diabetes mellitus type I).

Furthermore, the compounds and pharmaceutical compositions of the present invention are useful in the treatment of otological disorders, e.g., hearing loss.

The inventive compounds and pharmaceutical compositions may be administered by any suitable route, depending on the nature of the disorder to be treated, e.g. orally (as syrups, tablets, capsules, lozenges, controlled-release preparations, fast-dissolving preparations, lozenges, etc.); topically (as creams, ointments, lotions, nasal sprays or aerosols, etc.); by injection (subcutaneous, intradermic, intramuscular, intravenous, etc.) or by inhalation (as a dry powder, a solution, a dispersion, etc.).

### Detailed Description of Preparation Processes of Compounds of Formula I

The preparation of compounds of Formula I of the present invention can be carried out in sequential or convergent synthetic routes. Syntheses of the compounds of the invention are shown in the following schemes. The skills required for carrying out the reaction and purification of the resulting products are known to those skilled in the art. The substituents and indices used in the following description of the processes have the significance given herein unless indicated to the contrary. In more detail, the compounds of Formula I can be manufactured by the methods given below, by the methods given in the examples or by analogous methods. Appropriate reaction conditions for the individual reaction steps are known to a person skilled in the art. The reaction sequence is not limited to the one displayed in the schemes, however, depending on the starting materials and their respective reactivity the sequence of reaction steps can be freely altered. Starting materials are either commercially available or can be prepared by methods analogous to the methods given below, by methods described in the examples, or by methods known in the art.

The compounds of Formula I of the present invention can be prepared according to Schemes 1-6, below.

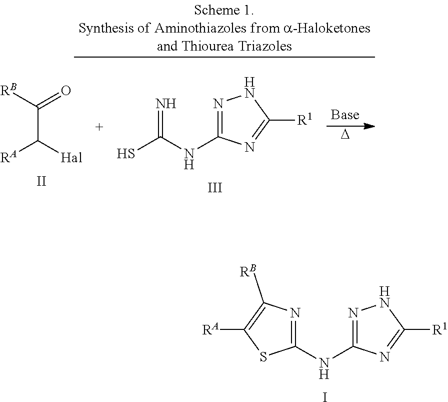

Compounds of Formula I of the present invention can be prepared by reacting α-haloketones of the Formula II with thiourea triazoles of the Formula III in an appropriate solvent, e.g., acetonitrile, preferably at elevated temperature in the presence of a base, preferably a nitrogen base, e.g., triethylamine. In Scheme 1, above, the “Hal” substituent of Formula II can be any halogen, preferably iodine or bromine, more preferably bromine. The condensation reaction produces compounds of Formula I which can be directly precipitated from the reaction mixture and purified by suitable procedures, preferably by washing.

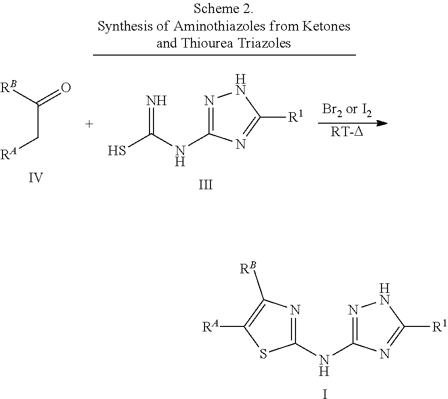

As shown above in Scheme 2, compounds of Formula I can be prepared from ketones of Formula IV and thiourea triazoles of the Formula III in a one-pot reaction by in situ halogenation of the Formula IV ketones with an appropriate halogenating reagent (preferably bromine or iodine) in an appropriate solvent (e.g., dioxane or pyridine). The one-pot halogenation-condensation reaction can proceed at room temperature or at elevated temperature.

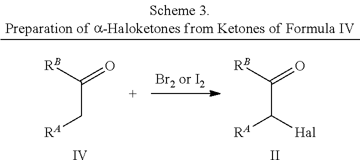

As shown in Scheme 3, the α-haloketones of the Formula II can be obtained for example by halogenation of ketones of Formula IV with an appropriate halogenating reagent in an appropriate solvent. In preferred embodiments the halogenating reagent is bromine, the solvent is acetic acid, and the reaction is carried out at room temperature. In other preferred embodiments, the halogenating reagent is iodine and the solvent is pyridine or dioxane.

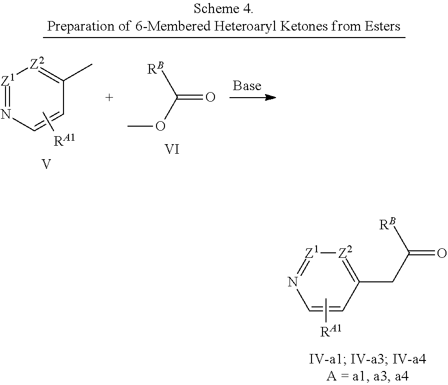

As shown in Scheme 4, α-(6-membered) heteroaryl ketones of the Formulae IV-a1, IV-a3, and IV-a4 can be prepared from 4-methyl substituted heteroaryl rings of the Formula V (a1: Z¹=Z²=CH; a3: Z¹=N, Z²=CH; a4: Z¹=CH, Z²=N) and heteroaryl carboxylate esters of the Formula VI. The 4-methyl heteroaryl rings can be deprotonated with an appropriate base (e.g., lithium bis(trimethylsilyl) amide; LiHMDS) in an appropriate solvent (e.g., THF) and reacted with the carboxylate of Formula VI in an acylation reaction to provide compounds of Formulae IV-a1, IV-a3, and IV-a4. In preferred embodiments, Z¹ and Z² are each independently N or CH; preferably the heteroaryl ring of Formulae V and IV is a 4-pyridinyl; 4-pyridazinyl; or 4-pyrimidinyl (i.e., wherein the heteroaryl R^{A} ring of Formula V is a one of rings a1, a3, or a4).

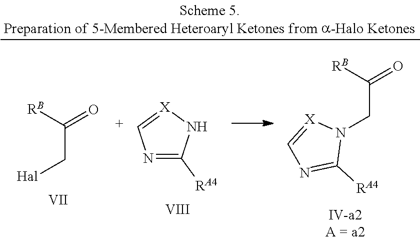

As shown in Scheme 5, α-(5-membered) heteroaryl ketones of the Formula IV-a2 (X=CH, N) can be prepared by reacting α-haloketones (e.g., α-bromoketones) of the Formula VII with a corresponding substituted imidazole or triazole of the Formula VIII in an appropriate solvent (e.g., acetonitrile). The substitution reaction provides compounds of Formula IV wherein X is CH or N (i.e., wherein the R^{A} ring is a 5-membered heteroaryl ring of the Formula a2).

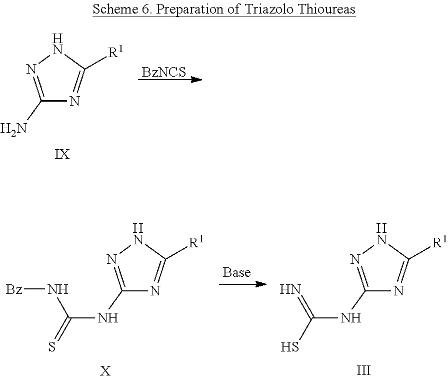

As shown in Scheme 6, triazole-thioureas of the Formula III can be prepared in two steps. In the first step, amino triazoles of the Formula IX are reacted with benzoyl isothiocyanate in an appropriate solvent (e.g., acetone) to provide a benzoyl-modified triazole-thiourea. In the second step, the benzoyl group is removed by treatment with an appropriate base, (e.g., KOH) in an appropriate solvent (e.g., methanol), preferably at elevated temperature (e.g., 60° C. or above) to provide triazole-thioureas of the Formula III.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

Abbreviations used in the following Examples and elsewhere herein are:
Conc. Concentrated
EtOAc Ethyl acetate
h hour
HPLC High pressure liquid chromatography
MeCN Acetonitrile
MeOH methanol
MTBE Methyl tert-butyl ether
r.t. room temperature
THF Tetrahydrofuran

### Preparative Example 1: Preparation of 2-Bromo-1-(2-Fluorophenyl)-2-(3-Fluoropyridin-4-Yl)Ethan-1-One Hydrogen Bromide (Intermediate II-1)

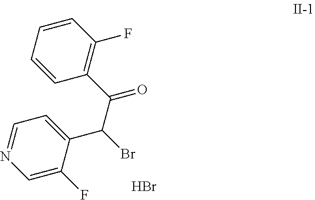 [SMILES: O=C(c1ccccc1F)C(Br)c1ccncc1F ; Br]

### Step 1: Synthesis of methyl 2-fluorobenzoate

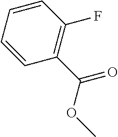

To a stirred suspension of 2-fluorobenzoic acid (30.0 g, 0.214 mol) and K₂CO₃ (32.5 g, 0.235 mol) in N,N-dimethylformamide (150 ml) was added iodomethane (45.6 g, 0.321 mol), and the reaction mixture was allowed to stir at r.t. overnight. Upon completion, the mixture was poured into water and extracted with 2-methoxy-2-methylpropane. The organic layer was washed with water and brine, dried, and evaporated in vacuo to obtain 31.5 g of methyl 2-fluorobenzoate (0.203 mol, 95% yield).

### Step 2: Synthesis of 1-(2-fluorophenyl)-2-(3-fluoropyridin-4-yl)ethan-1-one (Intermediate IV-1)

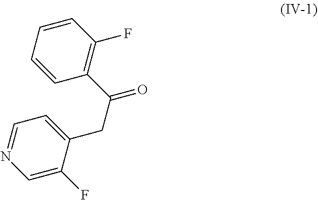 [SMILES: O=C(Cc1ccncc1F)c1ccccc1F]

Lithium bis(trimethylsilyl)amide (168 g, 23% in tetrahydrofuran/ethylbenzene, 0.232 mol) was added dropwise to a mixture of 3-fluoro-4-methylpyridine (21.4 g, 0.193 mol) and methyl 2-fluorobenzoate (31.2 g, 0.203 mol) in tetrahydrofuran (200 mL) at 0° C. After stirring for 1 h, the reaction mixture was allowed to warm to r.t. and stirred for 16 h, then triturated with hexane and filtered. The solid was dissolved in 3N HCl (300 mL). The solution was neutralized with saturated aqueous NaHCO₃ and extracted with ethyl acetate. The combined organic layers were washed with water followed by brine, dried with Na₂SO₄, and evaporated to obtain 1-(2-fluorophenyl)-2-(3-fluoropyridin-4-yl)ethan-1-one (Intermediate IV-1), (33.6 g, 75% yield) as a light yellow solid.

### Step 3: Synthesis of 2-bromo-1-(2-fluorophenyl)-2-(3-fluoropyridin-4-yl)ethan-1-one Hydrogen Bromide (Intermediate II-1)

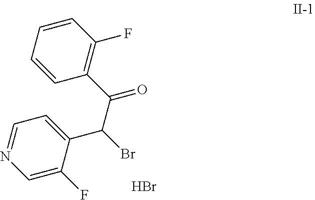 [SMILES: O=C(c1ccccc1F)C(Br)c1ccncc1F ; Br]

Bromine (5.5 g, 0.034 mol) was added to 1-(2-fluorophenyl)-2-(3-fluoropyridin-4-yl)ethan-1-one (8.0 g, 0.034 mol) in acetic acid (50 mL) and the mixture was stirred at room temperature for 1 hour. The reaction mixture was diluted with 2-methoxy-2-methylpropane, and the solid was filtered. The filter cake was washed with 2-methoxy-2-methylpropane and dried. The obtained 2-bromo-1-(2-fluorophenyl)-2-(3-fluoropyridin-4-yl)ethan-1-one hydrogen bromide (Intermediate II-1) was used in the next step without purification (11.2 g, 83% yield).

### Preparative Example 2: Preparation of N-(5-Cyclobutyl-1H-1,2,4-Triazol-3-Yl)Thiourea (Intermediate III-1)

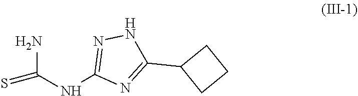 [SMILES: NC(=S)Nc1n[nH]c(C2CCC2)n1]

To a suspension of 5-cyclobutyl-1H-1,2,4-triazol-3-amine (1.37 g, 0.01 mol) in chloroform (50 mL) at 0° C. was slowly added benzoyl isothiocyanate (2.44 g, 0.0149 mol). The mixture was allowed to warm to r.t. and stirred for 8 h. The solids were collected (0.61 g, crude), washed with acetonitrile, dried, then, dissolved in methanol (20 mL). An aqueous solution (5 mL) of NaOH (0.243 g, 0.0061 mol) was added to this solution, and the mixture was stirred at 50° C. for another 2 h. The methanol was then removed under reduced pressure, and the residue was purified by preparative HPLC to obtain N-(5-cyclobutyl-1H-1,2,4-triazol-3-yl)thiourea (Intermediate III-1), (0.075 g, 2.5% yield).

### Preparative Example 3: Preparation of N-(5-Cyclopropyl-1H-1,2,4-Triazol-3-Yl)Thiourea (Intermediate III-2)

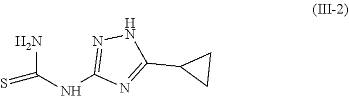 [SMILES: NC(=S)Nc1n[nH]c(C2CC2)n1]

### Step 1: Synthesis of N-[(5-cyclopropyl-1H-1,2,4-triazol-3-yl)carbamothioyl 9 benzamide

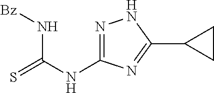

To a stirred mixture of 5-cyclopropyl-1H-1,2,4-triazol-3-amine (12.1 g, 97 mmol) in 200 mL of acetone was added benzoyl isothiocyanate (19.1 g, 116 mmol) at r.t. After the addition, the mixture was heated to reflux and stirred overnight. The resulting mixture was cooled to r.t. The precipitated solid was collected, washed with water and acetone, and dried to obtain N-[(5-cyclopropyl-1H-1,2,4-triazol-3-yl)carbamothioyl]-benzamide (11 g, 39%). [M+H]+: 288.08, found: 288.0.

### Step 2: Synthesis of N-(5-cyclopropyl-1H-1,2,4-triazol-3-yl)thiourea (Intermediate III-2)

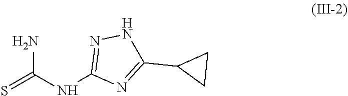 [SMILES: NC(=S)Nc1n[nH]c(C2CC2)n1]

To a suspension of N-[(5-cyclopropyl-1H-1,2,4-triazol-3-yl)carbamothioyl]benzamide in MeOH (200 mL) was added a solution of KOH (4.7 g, 84 mmol in 50 mL of water). The mixture was stirred at 60° C. for 16 h., cooled to r.t., and concentrated under the reduced pressure. The residue was diluted with water and EtOAc (100/100 mL), and acidified with concentrated HCl to pH=3. The precipitated solid was collected, washed with water, MeCN, and dried to afford N-(5-cyclopropyl-1H-1,2,4-triazol-3-yl)thiourea (Intermediate III-2), (4.5 g, 64%). [M+H]+: 184.06, found: 184.0.

### Preparative Example 4: Preparation of 3-[(3-Fluoropyridin-4-Yl)Acetyl]Benzonitrile (Intermediate IV-2)

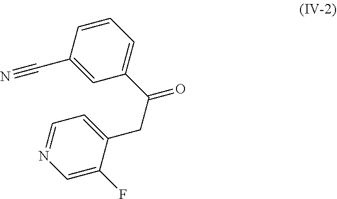 [SMILES: N#Cc1cccc(C(=O)Cc2ccncc2F)c1]

To a solution of methyl 3-cyanobenzoate (0.67 g, 4.1 mmol) and 3-fluoro-4-methylpyridine (0.55 g, 5.0 mmol) in THF (20 mL) was added LiHMDS (7 mL, 9.1 mmol, 20% solution in THF/ethylbenzene) at 0° C. under an argon atmosphere. The reaction mixture was warmed to r.t. and stirred overnight. Then, the mixture was diluted with MTBE. The precipitated solid was collected and washed with MTBE. The filter cake was dissolved in 1 N HCl, basified with saturated NaHCO₃, and extracted with EtOAc. The organic layer was washed with brine, dried, and evaporated in vacuo to obtain 3-[(3-fluoropyridin-4-yl)acetyl]benzonitrile (Intermediate IV-2) (0.63 g, 63%). [M+H]+: 241.07, found: 240.8

### Preparative Example 5: Preparation of 3-[(3-Methylpyridin-4-Yl)Acetyl]Benzonitrile (Intermediate IV-3)

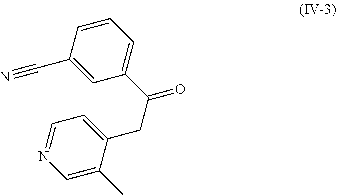 [SMILES: Cc1cnccc1CC(=O)c1cccc(C#N)c1]

To a solution methyl 3-cyanobenzoate (0.34 g, 2.1 mmol) and 3,4-dimethylpyridine (0.27 g, 2.5 mmol) in THF (10 mL) was added LiHMDS (4 mL, 5.2 mmol, 20% solution in THF/ethylbenzene) at 0° C. under an argon atmosphere. The reaction mixture was warmed to r.t., and stirred overnight. Then, the mixture was diluted with MTBE. The precipitated solid was collected and washed with MTBE. The filter cake was dissolved in 1 N HCl, basified with saturated NaHCO₃, and extracted with EtOAc. The organic layer was washed with brine, dried, and evaporated in vacuo to obtain 3-[(3-methylpyridin-4-yl)acetyl]benzonitrile (Intermediate IV-3), (0.23 g, 46%). [M+H]⁺: 236.09; found: 236.0.

### Preparative Example 6: Preparation of 3-[(2,5-Dimethylpyridin-4-Yl)Acetyl]Benzonitrile (Intermediate IV-4)

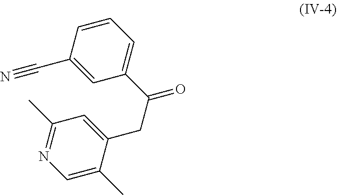 [SMILES: Cc1cc(CC(=O)c2cccc(C#N)c2)c(C)cn1]

To a solution of methyl 3-cyanobenzoate (0.53 g, 4.4 mmol) in THF (15 mL) was added LiHMDS (7 mL, 9.1 mmol, 20% solution in THF/ethylbenzene) at 0° C. under an argon atmosphere. After 30 min stirring at the same temperature, a solution of 2,4,5-trimethylpyridine (0.64 g, 4.4 mmol) in THF (2 mL) was added dropwise. The reaction mixture was warmed to r.t. and stirred overnight. Then, the mixture was quenched with MeOH, and concentrated under reduced pressure. The residue was taken up in MeOH and filtered. The filtrate was evaporated under the reduced pressure and purified by preparative HPLC to afford compound 3-[(2,5-dimethylpyridin-4-yl)acetyl]benzonitrile (Intermediate IV-4), (0.099 g, 10%). [M+H]⁺: 251.13; found: 251.0.

### Preparative Example 7: Preparation of 3-[(3-Chloropyridin-4-Yl)Acetyl]Benzonitrile (Intermediate IV-5)

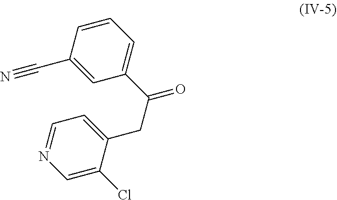 [SMILES: N#Cc1cccc(C(=O)Cc2ccncc2Cl)c1]

To a solution of methyl 3-cyanobenzoate (0.31 g, 2.3 mmol) and 3-chloro-4-methylpyridine (0.32 g, 2.5 mmol) in THF (10 mL) was added LiHMDS (3.3 mL, 4.3 mmol, 20% solution in THF/ethylbenzene) at 0° C. under an argon atmosphere. The reaction mixture was warmed to r.t., and stirred overnight. Then, the mixture was diluted with MTBE. The precipitated solid was collected and washed with MTBE. The filter cake was dissolved in 1 N HCl, basified with saturated NaHCO₃, and extracted with EtOAc. The organic layer was washed with brine, dried, and evaporated in vacuo to obtain 3-[(3-chloropyridin-4-yl)acetyl]benzonitrile (Intermediate IV-5), (0.45 g, 88%).

### Preparative Example 8: Preparation of 3-[(2,3-Dimethylpyridin-4-Yl)Acetyl]Benzonitrile (Intermediate IV-6)

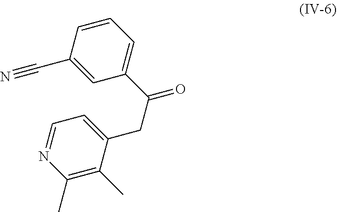 [SMILES: Cc1nccc(CC(=O)c2cccc(C#N)c2)c1C]

### Step 1: Synthesis of 4-Bromo-2,3-dimethylpyridine

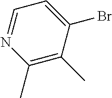

A mixture of 2,3-dimethylpyridin-4-ol (3.3 g 27 mmol) and phosphorus tribromide oxide (19.3 g, 67 mmol) was stirred at 130° C. for 4 h. under an argon atmosphere. The reaction mixture was poured onto ice, basified with aqueous NaOH, diluted with water, and extracted with diethyl ether. The organic layer was washed with brine, dried, and evaporated in vacuo to afford 4-bromo-2,3-dimethylpyridine (3.4 g, 68%). [M+H]⁺: 124.08, found: 122.0.

### Step 2: Synthesis of 2,3,4-trimethylpyridine

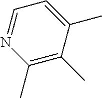

A mixture of 4-bromo-2,3-dimethylpyridine (1.54 g, 8.3 mmol), MeB(OH)₂ (1.977 g, 33 mmol), Na₂CO₃ (2.62 g, 27.7 mmol) in dioxane/water (20/5 mL) was bubbled with argon for five minutes before Pd(PPh₃)₄ (0.48 g) was added. The mixture was heated to 95° C. for 72 h. After completion of the reaction, the mixture was cooled to r.t, and concentrated under reduced pressure. The residue was partitioned between MTBE and water. The organic layer was washed with water and brine, dried, and evaporated in vacuo to obtain the crude residue, which was purified by flash column chromatography to afford 2,3,4-trimethylpyridine (0.49 g, 49%). [M+H]⁺: 122.09; found: 120.0.

### Step 3: Synthesis of 3-[(2,3-dimethylpyridin-4-yl)acetyl]benzonitrile (Intermediate IV-6)

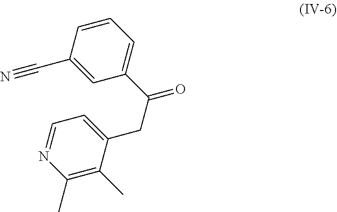 [SMILES: Cc1nccc(CC(=O)c2cccc(C#N)c2)c1C]

To a solution of 2,3,4-trimethylpyridine (0.46 g, 3.8 mmol) in THF (15 mL) was added LiHMDS (6.4 mL, 8.3 mmol, 20% solution in THF/ethylbenzene) at 0° C. under an argon atmosphere. After 30 min stirring at the same temperature, a solution of methyl 3-cyanobenzoate (0.61 g, 3.8 mmol) in THF (3 mL) was added dropwise. The reaction mixture was warmed to r.t. and stirred overnight. Then, the mixture was quenched with MeOH and concentrated under the reduced pressure. The residue was taken up in MeOH and filtered. The filtrate was evaporated under the reduced pressure and purified by preparative HPLC to afford 3-[(2,3-dimethylpyridin-4-yl)acetyl]benzonitrile (Intermediate IV-6), (0.055 g, 5.5%). [M+H]⁺: 251.13, found: 251.2.

### Preparative Example 9: Preparation of 3-[(2-Methylpyridin-4-Yl)Acetyl]Benzonitrile (Intermediate IV-7)

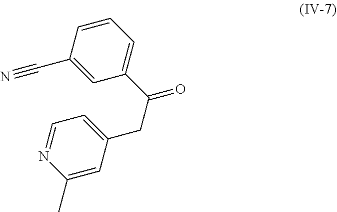 [SMILES: Cc1cc(CC(=O)c2cccc(C#N)c2)ccn1]

To a solution of 2,4-dimethylpyridine (1.02 g, 9.5 mmol) in THF (20 mL) was added LiHMDS (14.5 mL, 19 mmol, 20% solution in THF/ethylbenzene) at 0° C. under an argon atmosphere. After 30 min of stirring at 0° C., a solution of methyl 3-cyanobenzoate (1.02 g, 6.3 mmol) in THF (3 mL) was added dropwise. The reaction mixture was warmed to r.t. and stirred overnight. Then, the mixture was quenched with MeOH and concentrated. The residue was taken up in MeOH and filtered. The filtrate was evaporated under the reduced pressure and purified by preparative HPLC to afford 3-[(2-methylpyridin-4-yl)acetyl]benzonitrile (Intermediate IV-7), (0.255 g, 17%). [M+H]⁺: 237.11, found: 237.2.

### Preparative Example 10: Preparation of 3-[(2-Methyl-1H-Imidazol-1-Yl)Acetyl]Benzonitrile (Intermediate IV-8)

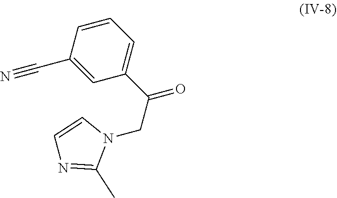 [SMILES: Cc1nccn1CC(=O)c1cccc(C#N)c1]

To a solution of 2-methyl-1H-imidazole (0.911 g, 211 mmol) in MeCN (10 mL) in an ice/water cooling bath was added 3-(bromoacetyl)benzonitrile (0.995 g, 4.4 mmol). The mixture was stirred at r.t. overnight, and the precipitate was filtered. The filtrate was evaporated under the reduced pressure. The residue was triturated with water and dried to obtain 3-[(2-methyl-1H-imidazol-1-yl)acetyl]benzonitrile (Intermediate IV-8), (0.62 g, 62%).

### Preparative Example 11: Preparation of N-1H-1,2,4-Triazol-3-Ylthiourea (Intermediate III-3)

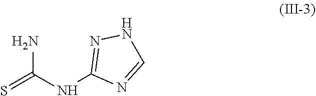

### Step 1: Synthesis of N-[(1H-1,2,4-triazol-3-yl)carbamothioyl]benzamide

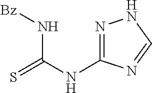

To a stirred mixture of 1H-1,2,4-triazol-3-amine (3.4 g, 40 mmol) in acetone (100 mL) was added benzoyl isothiocyanate (7.9 g, 48 mmol) at r.t. The mixture was heated to reflux and stirred overnight. Then, the resulting mixture was cooled to r.t. The precipitated solid was collected, washed with water and acetone and dried to afford N-[(1H-1,2,4-triazol-3-yl)carbamothioyl]benzamide (2.9 g, 29%).

### Step 2: Synthesis of N-1H-1,2,4-triazol-3-ylthiourea (Intermediate III-3)

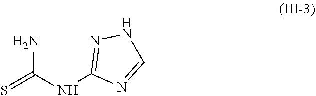

A solution of KOH (2.7 g, 47.5 mmol in 30 mL of water) was added to a suspension of N-[(1H-1,2,4-triazol-3-yl)carbamothioyl]benzamide (2.9 g, 11.8 mmol) in MeOH (50 mL). The reaction mixture was stirred at 60° C. for 16 h., cooled to r.t., and concentrated under reduced pressure. The residue was diluted with water and EtOAc (100/100 mL) and acidified with conc. HCl to pH=3. The precipitated solid was collected, washed with water and MeCN, and dried to afford N-1H-1,2,4-triazol-3-ylthiourea (Intermediate III-3) (1.2 g, 70%), which was used in the next step without purification.

### Preparative Example 12: Preparation of N-(5-Methyl-1H-1,2,4-Triazol-3-Yl)Thiourea (Intermediate III-4)

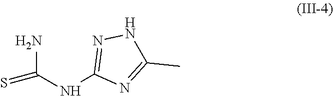

### Step 1: Synthesis of N-[(5-methyl-1H-1,2,4-triazol-3-yl)carbamothioyl]benzamide

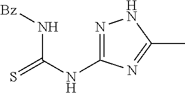

To a stirred mixture of 5-methyl-1H-1,2,4-triazol-3-amine (3.7 g, 38 mmol) in acetone (100 mL) was added benzoyl isothiocyanate (9.37 g, 57 mmol) at r.t. The mixture was heated to reflux and stirred overnight. Then, the resulting mixture was cooled to r.t. The precipitated solid was collected, washed with water and acetone, and dried to afford N-[(5-methyl-1H-1,2,4-triazol-3-yl)carbamothioyl]benzamide (4.9 g, 49%). [M+H]⁺: 262.07, found: 262.0.

### Step 2: Synthesis of N-(5-methyl-1H-1,2,4-triazol-3-yl)thiourea (Intermediate III-4)

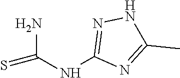

A solution of KOH (4.3 g, 76 mmol in 50 mL of water) was added to a suspension of N-[(5-methyl-1H-1,2,4-triazol-3-yl)carbamothioyl]benzamide (4.9 g, 19 mmol) in MeOH (100 mL). The reaction mixture was stirred at 60° C. for 16 h., cooled to r.t., and concentrated under reduced pressure. The residue was diluted with water and EtOAc (100/100 mL) and acidified with conc. HCl to pH=3. The precipitated solid was collected, washed with water and MeCN, and dried to afford N-(5-methyl-1H-1,2,4-triazol-3-yl)thiourea (Intermediate III-4), (2.1 g, 70%), which was used in the next step without purification.

### Preparative Example 13: Preparation of N-[5-(Hydroxymethyl)-1H-1,2,4-Triazol-3-Yl]Thiourea (Intermediate III-5)

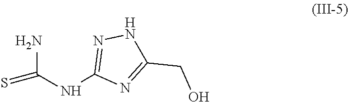 [SMILES: NC(=S)Nc1n[nH]c(CO)n1]

### Step 1: Synthesis of N-{[5-(hydroxymethyl)-1H-1,2,4-triazol-3-yl]carbamothioyl}benzamide

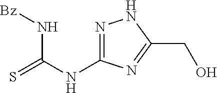

To a stirred mixture of (3-amino-1H-1,2,4-triazol-5-yl)methanol (1.65 g, 14.5 mmol) in acetone (70 mL) was added benzoyl isothiocyanate (3.53 g, 21.7 mmol) at r.t. The mixture was then heated to reflux and stirred overnight. Then, the resulting mixture was cooled to r.t. The precipitated solid was collected, washed with water and acetone, and dried to afford N-{[5-(hydroxymethyl)-1H-1,2,4-triazol-3-yl]carbamothioyl}benzamide (3.2 g, 67%). [M+H]⁺: 278.06, found: 277.0.

### Step 2: Synthesis of N-[5-(hydroxymethyl)-1H-1,2,4-triazol-3-yl]thiourea (Intermediate III-5)

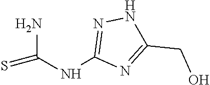 [SMILES: NC(=S)Nc1n[nH]c(CO)n1]

A solution of KOH (2.6 g, 46 mmol in 20 mL of water) was added to a suspension of N-{[5-(hydroxymethyl)-1H-1,2,4-triazol-3-yl]carbamothioyl}benzamide (3.2 g, 11.5 mmol) in MeOH (40 mL). The reaction mixture was stirred at 60° C. for 16 h., cooled to r.t. and concentrated under reduced pressure. The residue was diluted with water and EtOAc (50/50 mL) and acidified with conc. HCl to pH=3. The precipitated solid was collected, washed with water and MeCN, and dried to afford N-[5-(hydroxymethyl)-1H-1,2,4-triazol-3-yl]thiourea (Intermediate III-5), (0.9 g, 45%) which was used in the next step without purification.

### Preparative Example 14: Preparation of 3-[(1H-1,2,4-Triazol-1-Yl)Acetyl]Benzonitrile (Intermediate IV-9)

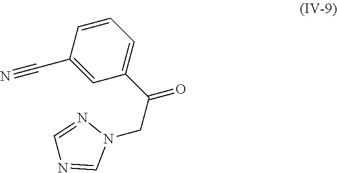 [SMILES: N#Cc1cccc(C(=O)Cn2cncn2)c1]

To a suspension of 1, 2, 4-triazole (0.366 g, 5.3 mmol) and K₂CO₃ (0.798 g, 5.8 mmol) in an ice/water bath was added 3-(bromoacetyl)benzonitrile (1.055 g, 4.7 mmol) in MeCN (10 mL). The reaction mixture was stirred at r.t. overnight. The precipitate was filtered. The filtrate was evaporated under reduced pressure. The residue was triturated with water and dried to obtain 3-[(1H-1,2,4-triazol-1-yl)acetyl]benzonitrile (Intermediate IV-9), (0.61 g, 61%), which was used in the next step without purification.

### Preparative Example 15: Preparation of 3-[(2-Ethyl-1H-Imidazol-1-Yl)Acetyl]Benzonitrile (Intermediate IV-10)

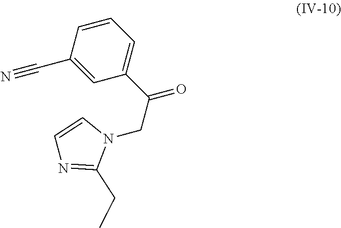 [SMILES: CCc1nccn1CC(=O)c1cccc(C#N)c1]

To a suspension of 2-ethyl-1H-imidazole (0.482 g, 5 mmol) and K₂CO₃ (0.798 g, 5.8 mmol) in an ice/water bath was added 3-(bromoacetyl)benzonitrile (0.936 g, 4.2 mmol) in MeCN (10 mL). The reaction mixture was stirred at r.t. overnight. The precipitate was filtered. The filtrate was evaporated under reduced pressure. The residue was triturated with water and dried to obtain 3-[(2-ethyl-1H-imidazol-1-yl)acetyl]benzonitrile (Intermediate IV-10), (0.62 g, 62%), which was used in the next step without purification.

### Preparative Example 16: Preparation of 3-{[2-(Hydroxymethyl)-1H-Imidazol-1-Yl]Acetyl}Benzonitrile (Intermediate IV-11)

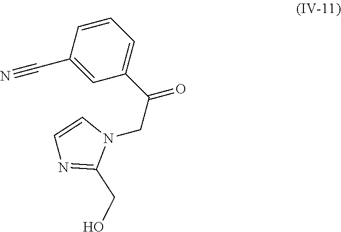 [SMILES: N#Cc1cccc(C(=O)Cn2ccnc2CO)c1]

To a suspension of (1H-imidazol-2-yl) methanol (0.488 g, 5 mmol) and K₂CO₃ (0.745 g, 5.4 mmol) in an ice/water bath was added 3-(bromoacetyl)benzonitrile (0.929 g, 4.1 mmol) in MeCN (10 mL). The reaction mixture was stirred at r.t. overnight. The precipitate was filtered. The filtrate was evaporated under reduced pressure, diluted with water, and extracted with EtOAc. The organic layer was washed with water and brine, dried, and evaporated in vacuo to obtain crude 3-{[2-(hydroxymethyl)-1H-imidazol-1-yl]acetyl}benzonitrile (Intermediate IV-11), (0.62 g, 62%), which was used in the next step without purification.

### Preparative Example 17: Preparation of 4-Fluoro-3-[(2-Methyl-1H-Imidazol-1-Yl)Acetyl]Benzonitrile (Intermediate IV-12)

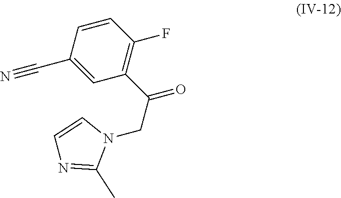 [SMILES: Cc1nccn1CC(=O)c1cc(C#N)ccc1F]

### Step 1: Synthesis of 3-Acetyl-4-fluorobenzonitrile

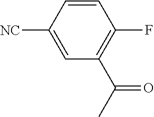

A mixture of 1-(5-bromo-2-fluorophenyl)ethan-1-one (3.06 g, 14 mmol), Zn(CN)₂ (2.48 g, 21 mmol) in DMF (15 mL) was degassed, and Pd(PPh₃)₄ was added. The mixture was heated to 90° C. overnight. The resulting mixture was cooled to r.t., poured into water, and extracted with MTBE. The organic layer was washed with water and brine, dried, and evaporated in vacuo. The residue was purified by flash column chromatography to afford 3-acetyl-4-fluorobenzonitrile (1.15 g, 50%).

### Step 2: Synthesis of 3-(Bromoacetyl)-4-fluorobenzonitrile

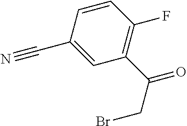 [SMILES: N#Cc1ccc(F)c(C(=O)CBr)c1]

3-Acetyl-4-fluorobenzonitrile (0.674 g, 4.1 mmol) was dissolved in CHCl₃ (20 mL). To the solution was added Br₂ (0.759 g, 4.7 mmol). The mixture was stirred at r.t. for 2 h., and diluted with a sat. NaHCO₃ solution. The organic layer was separated, washed with water and brine, dried, and evaporated in vacuo to afford 3-(bromoacetyl)-4-fluorobenzonitrile (0.89 g, 89%). [M+H]⁺: 241.95, found: 242.0.

### Step 3: 4-fluoro-3-[(2-methyl-1H-imidazol-1-yl)acetyl]benzonitrile (Intermediate IV-12)

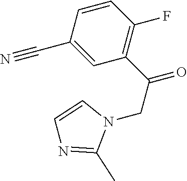 [SMILES: Cc1nccn1CC(=O)c1cc(C#N)ccc1F]

To a suspension of 2-methyl-1H-imidazole (0.456 g, 5.5 mmol) and K₂CO₃ (0.818 g, 5.9 mmol), in an ice/water bath was added 3-(bromoacetyl)-4-fluorobenzonitrile (0.896 g, 3.7 mmol) in MeCN (10 mL). The reaction mixture was stirred at r.t. overnight. The precipitate was filtered. The filtrate was evaporated under reduced pressure, diluted with water, and extracted with EtOAc. The organic layer was washed with water and brine, dried, and evaporated in vacuo to obtain crude 4-fluoro-3-[(2-methyl-1H-imidazol-1-yl)acetyl]benzonitrile (Intermediate IV-12), (0.54 g, 60%), which was used in the next step without purification.

### Preparative Example 18: Preparation of 3-[(5-Methylpyridazin-4-Yl)Acetyl]Benzonitrile (Intermediate IV-13)

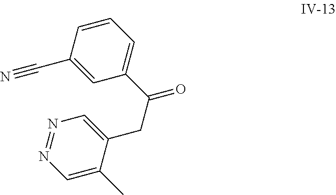 [SMILES: Cc1cnncc1CC(=O)c1cccc(C#N)c1]

### Step 1: Synthesis of 4,5-dimethylpyridazine

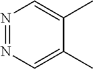

To a solution of compound 3,6-dichloro-4,5-dimethylpyridazine (3.3 g, 18.5 mmol) in THF (50 mL), DIEA (5.26 g, 40.7 mmol) and Pd/C (10%, 0.98 g, 9.2 mmol) were added. The reaction mixture was degassed and backfilled with hydrogen, and then was stirred at r.t. under a hydrogen atmosphere until full consumption of the starting material. Upon completion, the Pd/C catalyst was removed by filtration, and the filtrate was evaporated under reduced pressure. The residue was taken up in DCM (100 mL) and washed with 5% NH₄OH (50 mL). The organic layer was dried over Na₂SO₄ and evaporated to dryness to obtain 4,5-dimethylpyridazine (1.4 g, 70% yield).

### Step 2: Preparation of 3-[(5-methylpyridazin-4-yl)acetyl]benzonitrile (Intermediate IV-13)

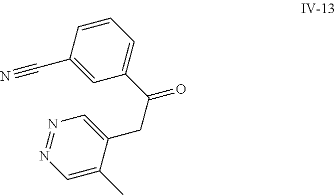 [SMILES: Cc1cnncc1CC(=O)c1cccc(C#N)c1]

To a solution of 4,5-dimethylpyridazine (501 mg, 4.6 mmol) in THF (15 mL), LiHMDS (4.2 mL, 5.0 mmol, 20% solution in THF/ethylbenzene) was added at 0° C. under an argon atmosphere. After 30 min of stirring at 0° C., a solution of methyl 3-cyanobenzoate (679 mg, 4.2 mmol) in THF (2 mL) was added dropwise. The reaction mixture was warmed to r.t. and stirred overnight. The mixture was diluted with hexane. The resulting solid was collected and dissolved in 1 N HCl. The aqueous layer was washed with MTBE, basified by NaHCO₃, and extracted with EtOAc. The organic layer was washed with brine, dried over Na₂SO₄, and evaporated in vacuo to obtain 3-[(5-methylpyridazin-4-yl)acetyl]benzonitrile (Intermediate IV-13) (0.3 g, 30% yield). [M+H]⁺: 238.09, found: 238.4.

### Preparative Example 19: Preparation of 3-[(Pyrimidin-4-Yl)Acetyl]Benzonitrile (Intermediate IV-14)

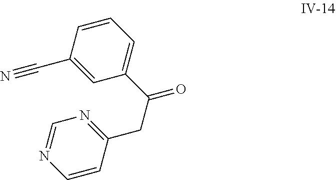 [SMILES: N#Cc1cccc(C(=O)Cc2ccncn2)c1]

To a solution of 4-methylpyrimidine (464 mg, 4.93 mmol) in THF (15 mL), LiHMDS (5.6 mL, 6.72 mmol, 20% solution in THF/ethylbenzene) was added at 0° C. under an argon atmosphere. After 30 min of stirring at 0° C., a solution of methyl 3-cyanobenzoate (682 mg, 4.23 mmol) in THF (2 mL) was added dropwise. The reaction mixture was warmed to r.t. and stirred overnight. The mixture was diluted with hexane. The solid was collected, taken up in EtOAc and washed with 1 N HCl and brine, then dried over Na₂SO₄ and evaporated in vacuo to give 3-[(pyrimidin-4-yl)acetyl]benzonitrile (Intermediate IV-14) (0.2 g, 20% yield). [M+H]⁺: 223.07, found: 223.09.

### Preparative Example 20: Preparation of 3-[(Pyridazin-4-Yl)Acetyl]Benzonitrile (Intermediate IV-15)

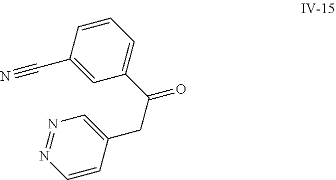 [SMILES: N#Cc1cccc(C(=O)Cc2ccnnc2)c1]

To a solution of 4-methylpyridazine (464 mg, 4.93 mmol) in THF (15 mL), LiHMDS (5.6 mL, 6.72 mmol, 20% solution in THF/ethylbenzene) was added at 0° C. under an argon atmosphere. After 30 min of stirring at 0° C., a solution of methyl 3-cyanobenzoate (722 mg, 4.5 mmol) in THF (2 mL) was added dropwise. The reaction mixture was warmed to r.t. and stirred overnight. The mixture was diluted with hexane. The solid was collected and dissolved in 1 N HCl. The aqueous layer was washed with MTBE, basified by NaHCO₃, and extracted with EtOAc. The organic layer was washed with brine, dried over Na₂SO₄, and evaporated in vacuo to obtain 3-[(pyridazin-4-yl)acetyl]benzonitrile (Intermediate IV-15) (0.34 g, 34% yield). [M+H]⁺: 224.08, found: 224.0.

### Preparative Example 21: Preparation of 3-[(Pyridin-4-Yl)Acetyl]Benzonitrile (Intermediate IV-16)

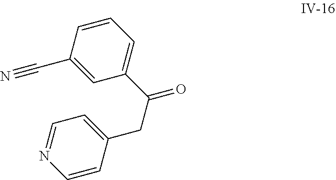 [SMILES: N#Cc1cccc(C(=O)Cc2ccncc2)c1]

To a solution of 4-methylpyridine (461 mg, 4.95 mmol) in THF (15 mL), LiHMDS (4.9 mL, 5.85 mmol, 20% solution in THF/ethylbenzene) was added at 0° C. under an argon atmosphere. After 30 min of stirring at 0° C., a solution of methyl 3-cyanobenzoate (725 mg, 4.5 mmol) in THF (2 mL) was added dropwise. The reaction mixture was warmed to r.t. and stirred overnight. The mixture was diluted with hexane. The solid was collected and dissolved in 1 N HCl. The aqueous layer was washed with MTBE, basified by NaHCO₃, and extracted with EtOAc. The organic layer was washed with brine, dried over Na₂SO₄, and evaporated in vacuo to obtain 3-[(pyridin-4-yl)acetyl]benzonitrile (Intermediate IV-16) (0.6 g, 60% yield). [M+H]⁺: 223.08, found: 223.02.

### Preparative Example 22: Preparation of 3-[(5-Methylpyrimidin-4-Yl)Acetyl]Benzonitrile (Intermediate IV-17)

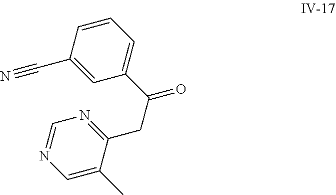 [SMILES: Cc1cncnc1CC(=O)c1cccc(C#N)c1]

To a solution of 4,5-dimethylpyrimidine (410 mg, 3.8 mmol) in THF (15 mL), LiHMDS (3.8 mL, 4.55 mmol, 20% solution in THF/ethylbenzene) was added at 0° C. under an argon atmosphere. After 30 min of stirring at 0° C., a solution of methyl 3-cyanobenzoate (611 mg, 3.8 mmol) in THF (2 mL) was added dropwise. The reaction mixture was warmed to r.t. and stirred overnight. The mixture was diluted with hexane. The solid was collected, taken up in EtOAc, and washed with 1 N HCl and brine, then dried over Na₂SO₄ and evaporated in vacuo to obtain 3-[(5-methylpyrimidin-4-yl)acetyl]benzonitrile (Intermediate IV-17) (0.15 g, 17% yield). [M+H]⁺: 238.09, found: 238.0.

### Preparative Example 23: Preparation of 3-[(1-Methyl-6-Oxo-1,6-Dihydropyridin-3-Yl)Acetyl]Benzonitrile (Intermediate IV-18)

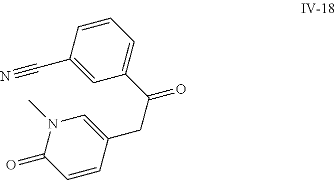 [SMILES: Cn1cc(CC(=O)c2cccc(C#N)c2)ccc1=O]

### Step 1: Preparation of dimethyl [(3-cyanophenyl)(hydroxy)methyl]phosphonate

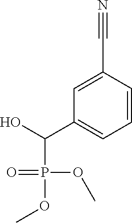 [SMILES: COP(=O)(OC)C(O)c1cccc(C#N)c1]

3-Formylbenzonitrile (1.09 g, 8.29 mmol) was dissolved in ethyl acetate (15 mL). Then, triethylamine (1.26 g, 12.4 mmol) was added, followed by dimethylphosphite (1.19 g, 10.8 mmol) and the reaction mixture was stirred at room temperature for 2 h, then diluted with ethyl acetate (350 mL) and washed with saturated aqueous NH₄Cl (2×30 mL). The organic layer was dried over Na₂SO₄ and evaporated under reduced pressure to obtain 1.7 g of dimethyl [(3-cyanophenyl)(hydroxy)methyl]phosphonate (85% yield). [M+H]⁺: 241.05, found: 242.2.

### Step 2: Preparation of dimethyl {(3-cyanophenvl)[(oxan-2-yl)oxy]methyl}phosphonate

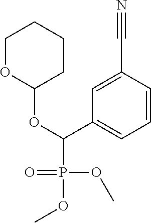 [SMILES: COP(=O)(OC)C(OC1CCCCO1)c1cccc(C#N)c1]

3,4-Dihydro-2H-pyran (1.25 g, 14.8 mmol) and p-toluenesulfonic acid (37 mg, 0.2 mmol) were added to a solution of dimethyl [(3-cyanophenyl)(hydroxy)methyl]phosphonate (1.7 g, 7.07 mmol) in dry toluene (30 mL) and the reaction mixture was stirred under a nitrogen atmosphere at 50° C. for 3 h. The solvent was then removed under vacuum and the residue was taken up with MTBE. The organic layer was washed with a saturated NaHCO₃ solution and brine then dried over Na₂SO₄. The filtrate was evaporated to dryness to give dimethyl {(3-cyanophenyl)[(oxan-2-yl)oxy]methyl}phosphonate (2.2 g, 96% yield). [M+H]⁺: 325.11, found: 324.9.

### Step 3: Preparation of 3-[(1-methyl-6-oxo-1,6-dihydropyridin-3-yl)acetyl]benzonitrile (Intermediate IV-18)

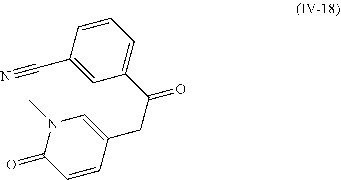 [SMILES: Cn1cc(CC(=O)c2cccc(C#N)c2)ccc1=O]

Sodium hydride (187 mg, 4.46 mmol) was added to a solution of dimethyl {(3-cyanophenyl)[(oxan-2-yl)oxy]methyl}phosphonate (967 mg, 3.0 mmol) in dry THF (20 mL), and the mixture was stirred at r. t. for 15 min. Next, 1-methyl-6-oxo-1,6-dihydropyridine-3-carbaldehyde (489 mg, 3.5 mmol) was added and the reaction mixture was stirred at 50° C. for 3 h under a nitrogen atmosphere. The resulting mixture was cooled to r.t., distilled water was slowly added, and the solvent was removed under reduced pressure. The water layer was extracted with MTBE, and the organic layer was washed with water and brine, dried over Na₂SO₄ and filtered. The filtrate was evaporated to dryness to give crude THP-protected intermediate, which was taken up in ether (10 mL) and sat. ethereal HCl was added (5 mL) and the obtained mixture was stirred for 1 h at ambient temperature. The precipitated solid was collected and dried to give 3-[(1-methyl-6-oxo-1,6-dihydropyridin-3-yl)acetyl]benzonitrile (0.35 g, 47% yield). [M+H]⁺: 252.09, found: 252.1.

### Preparative Example 24: Preparation of N-(5-Ethyl-1H-1,2,4-Triazol-3-Yl)Thiourea (Intermediate III-6)

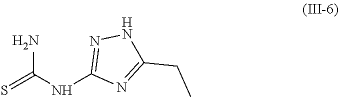

### Step 1: N-[(5-ethyl-1H-1,2,4-triazol-3-yl)carbamothioyl]benzamide

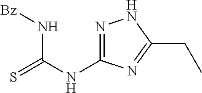

To a stirred mixture of 5-ethyl-1H-1,2,4-triazol-3-amine (1.2 g, 10.9 mmol) in acetone (30 mL), benzoyl isothiocyanate (2.67 g, 16.3 mmol) was added at r.t. After the addition, the mixture was heated to reflux and stirred overnight. The reaction mixture was cooled to r.t. and evaporated under reduced pressure to afford crude N-[(5-ethyl-1H-1,2,4-triazol-3-yl)carbamothioyl]benzamide (3.2 g), which was used in the next step without purification.

### Step 2: Preparation of N-(5-ethyl-1H-1,2,4-triazol-3-yl)thiourea (intermediate III-6)

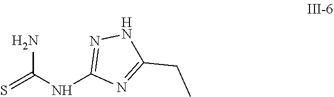

A solution of KOH (1.97 g, 35 mmol in 15 mL of water) was added to a suspension of N-[(5-ethyl-1H-1,2,4-triazol-3-yl)carbamothioyl]benzamide (3.2 g, 11 mmol) in MeOH (40 mL). The reaction mixture was stirred at 60° C. for 16 h. The mixture was cooled to r.t. and concentrated under reduced pressure. The residue was diluted with water and EtOAc (50/50 mL) and acidified with conc. HCl to pH=3. The layers were separated. The organic layer was dried over Na₂SO₄, evaporated under reduced pressure, and triturated with IPA/Hex to afford N-(5-ethyl-1H-1,2,4-triazol-3-yl)thiourea (Intermediate III-6) (0.2 g, 10% yield over 2 steps). [M+H]+: 172.06, found 172.1

### Preparative Example 25: Preparation of Compounds of Formula I from Intermediates II and III

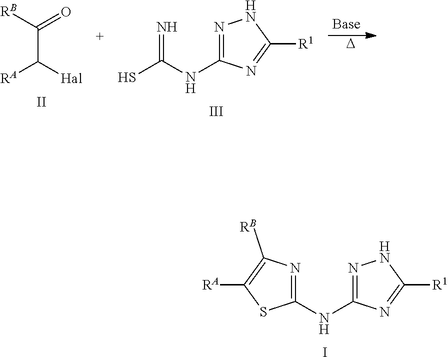

A mixture of haloketone intermediate of Formula II (0.144 g, 0.0004 mol; 1 eq), thiourea triazole intermediate of Formula III (0.072 g, 0.0004 mol; 1 eq), and triethylamine (0.111 g, 0.0011 mol; 2.75 eq) in acetonitrile (2 ml) was heated to 60° C. and stirred overnight. The precipitated solid was collected, washed with water and acetonitrile, and dried to obtain the target compounds of Formula I.

### Preparative Example 26: Preparation of Compounds of Formula I from Intermediates III and IV Via In Situ Halogenation and Precipitation

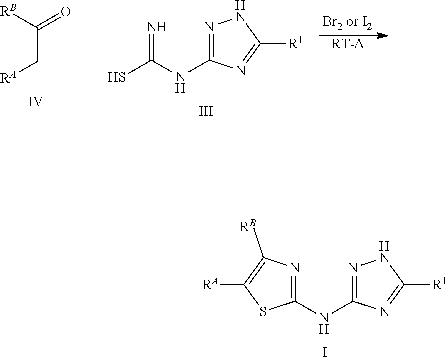

Ketone intermediate of Formula IV (0.63 g, 2.6 mmol; 1 eq) was dissolved in dioxane (10 mL), followed by addition of Br₂ (0.42 g, 2.6 mmol; 1 eq) at r.t. The mixture was stirred at r.t. overnight, and thiourea triazole intermediate of Formula III (0.48 g, 2.6 mmol; 1 eq) and EtOH (10 mL) were added. The resulting mixture was stirred overnight. The precipitated solid was collected, washed with saturated NaHCO₃, water, cold MeCN, and dried to obtain the target compound of Formula I. No additional purification was necessary.

### Preparative Example 27: Preparation of Compounds of Formula I from Intermediates III and IV Via In Situ Halogenation and Chromatography

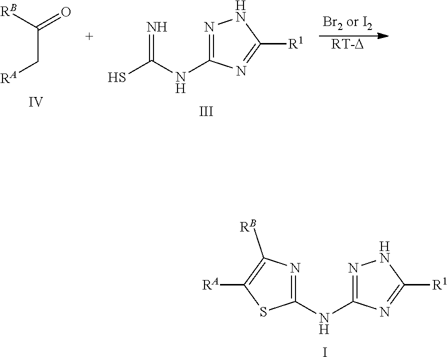

Ketone intermediate of Formula IV (0.091 g, 0.36 mmol; 1 eq) was dissolved in dioxane (2 mL), followed by the addition of Br₂ (0.070 g, 0.44 mmol; 1.2 eq) at r.t. The mixture was stirred at r.t. overnight, and thiourea triazole intermediate of Formula III (0.066 g, 0.36 mmol; 1 eq) and EtOH (2 mL) were added. The resulting mixture was stirred overnight. After the reaction was completed, the solution was basified with NH₄OH, and purified by preparative HPLC to afford to obtain the target compound of Formula I.

### Preparative Example 28: Preparation of Compounds of Formula I from Intermediates IV and III when R^{A} is A2

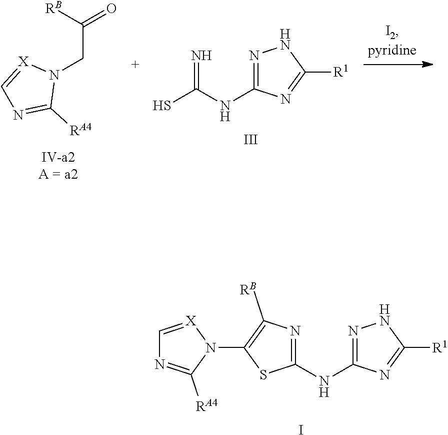

A mixture of ketone intermediate of Formula IV with A=a2 (0.174 g, 0.77 mmol; 1 eq), and thiourea triazole intermediate of Formula III (0.142 g, 0.77 mmol; 1 eq), iodine (0.196 g, 0.77 mmol; 1 eq) and pyridine (2 mL) was stirred at r.t. overnight. The resulting mixture was diluted with water (50 mL). The solid obtained was filtered, stirred in water (50 mL) for 30 minutes, and filtered again. The crude product was purified by preparative HPLC to afford title compounds I with A=a2.

### Example 1: Preparation of 5-Cyclobutyl-N-[4-(2-Fluorophenyl)-5-(3-Fluoropyridin-4-Yl)-1,3-Thiazol-2-Yl]-1H-1,2,4-Triazol-3-Amine (Compound 1)

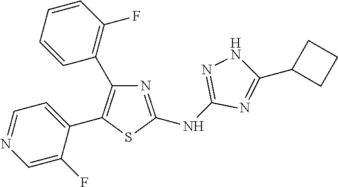 [SMILES: Fc1ccccc1-c1nc(Nc2n[nH]c(C3CCC3)n2)sc1-c1ccncc1F]

Compound 1 was prepared from 2-bromo-1-(2-fluorophenyl)-2-(3-fluoropyridin-4-yl)ethan-1-one hydrogen bromide (Intermediate II-1) and N-(5-cyclobutyl-1H-1,2,4-triazol-3-yl)thiourea (Intermediate III-1) according to Preparative Example 25. Scale: 0.4 mmol.

Specifically, 2-bromo-1-(2-fluorophenyl)-2-(3-fluoropyridin-4-yl)ethan-1-one hydrogen bromide (0.124 g; 0.4 mmol) and N-(5-cyclobutyl-1H-1,2,4-triazol-3-yl)thiourea (79 mg; 0.4 mmol) were added to a solution of triethylamine (0.111 g; 1.1 mmol) in acetonitrile (2 mL). The mixture was heated to 60° C. and stirred overnight. The precipitated solid was collected, washed with water and acetonitrile, and dried under vacuum to obtain Compound 1 (5-Cyclobutyl-N-[4-(2-fluorophenyl)-5-(3-fluoropyridin-4-yl)-1,3-thiazol-2-yl]-1H-1,2,4-triazol-3-amine; 0.046 g; 31% yield). LCMS [M+H]⁺: 411.2. ¹H NMR (DMSO-d₆, 500 MHz): δ (ppm) 1.90 (m, 1H), 2.03 (m, 1H), 2.29 (m, 4H), 3.59 (m, 1H), 7.18 (m, 2H), 7.26 (t, 1H), 7.43 (m, 1H), 7.52 (m, 1H), 8.27 (s, 1H), 8.54 (s, 1H), 11.61 (s, 1H), 13.36 (s, 1H).

### Example 2: Preparation of 3-{2-[(5-Cyclopropyl-1H-1,2,4-Triazol-3-Yl)Amino]-5-(3-Fluoropyridin-4-Yl)-1,3-Thiazol-4-Yl}Benzonitrile (Compound 2)

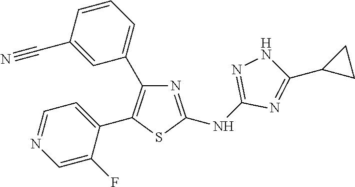 [SMILES: N#Cc1cccc(-c2nc(Nc3n[nH]c(C4CC4)n3)sc2-c2ccncc2F)c1]

Compound 2 was prepared from 3-[(3-fluoropyridin-4-yl)acetyl]benzonitrile (Intermediate IV-2), Br₂, and N-(5-cyclopropyl-1H-1,2,4-triazol-3-yl)thiourea (Intermediate III-2) according to Preparative Example 26. Scale: 2.6 mmol.

Specifically, 3-[(3-fluoropyridin-4-yl)acetyl]benzonitrile (0.63 g, 2.6 mmol) was dissolved in dioxane (10 mL), followed by addition of Br₂ (0.42 g, 2.6 mmol) at r.t. The mixture was stirred at r.t. overnight, and N-(5-cyclopropyl-1H-1,2,4-triazol-3-yl)thiourea (0.48 g, 2.6 mmol) and EtOH (10 mL) were added. The resulting mixture was stirred overnight. The precipitated solid was collected, washed with saturated NaHCO₃, water, cold MeCN, and dried under vacuum to obtain Compound 2 (3-{2-[(5-cyclopropyl-1H-1,2,4-triazol-3-yl)amino]-5-(3-fluoropyridin-4-yl)-1,3-thiazol-4-yl}benzonitrile; 0.32 g; 30% yield). LCMS [M+H]⁺: 404.2. ¹H NMR (500 MHz, DMSO-d₆): δ (ppm) 0.91 (m, 2H), 1.03 (m, 2H), 2.00 (m, 1H), 7.37 (t, 1H), 7.53 (t, 1H), 7.66 (d, 1H), 7.79 (d, 1H), 7.82 (s, 1H), 8.38 (d, 1H), 8.58 (s, 1H), 11.55 (s, 1H), 13.33 (s, 1H).

### Example 3: Preparation of 3-{2-[(5-Cyclopropyl-1H-1,2,4-Triazol-3-Yl)Amino]-5-(3-Methylpyridin-4-Yl)-1,3-Thiazol-4-Yl}Benzonitrile (Compound 3)

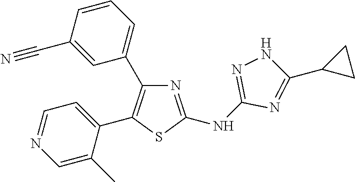 [SMILES: Cc1cnccc1-c1sc(Nc2n[nH]c(C3CC3)n2)nc1-c1cccc(C#N)c1]

Compound 3 was prepared from 3-[(3-methylpyridin-4-yl)acetyl]benzonitrile (Intermediate IV-3), Br₂, and N-(5-cyclopropyl-1H-1,2,4-triazol-3-yl)thiourea (Intermediate III-2) according to Preparative Example 26. Scale: 1 mmol. 0.09 g; 23% yield. [M+H]⁺: 400.0. ¹H NMR (400 MHz, DMSO-d₆): δ (ppm) 0.91 (m, 2H), 1.03 (m, 2H), 1.98 (m, 1H), 2.00 (s, 3H), 7.32 (d, 1H), 7.47 (t, 1H), 7.53 (d, 1H), 7.72 (m, 2H), 8.44 (d, 1H), 8.52 (s, 1H), 11.41 (s, 1H), 13.31 (s, 1H).

### Example 4: Preparation of 3-{2-[(5-Cyclopropyl-1H-1,2,4-Triazol-3-Yl)Amino]-5-(2,5-Dimethylpyridin-4-Yl)-1,3-Thiazol-4-Yl}Benzonitrile (Compound 4)

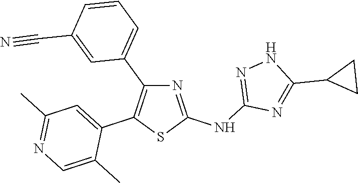 [SMILES: Cc1cc(-c2sc(Nc3n[nH]c(C4CC4)n3)nc2-c2cccc(C#N)c2)c(C)cn1]

Compound 4 was prepared from 3-[(2,5-dimethylpyridin-4-yl)acetyl]benzonitrile (Intermediate IV-4) Br₂, and N-(5-cyclopropyl-1H-1,2,4-triazol-3-yl)thiourea (Intermediate III-2) according to Preparative Example 27. Scale: 0.36 mmol.

Specifically, 3-[(2,5-dimethylpyridin-4-yl)acetyl]benzonitrile (0.091 g, 0.36 mmol) was dissolved in dioxane (2 mL), followed by the addition of Br₂ (0.070 g, 0.44 mmol) at r.t. The mixture was stirred at r.t. overnight, and N-(5-cyclopropyl-1H-1,2,4-triazol-3-yl)thiourea (0.066 g, 0.36 mmol) and EtOH (2 mL) were added. The resulting mixture was stirred overnight. After the reaction was completed, the solution was quenched with NH₄OH and purified by preparative HPLC to afford Compound 4 (0.022 g; 150% yield). [M+H]⁺: 414.2. ¹H NMR (500 MHz, DMSO-d₆): δ (ppm) 0.89 (m, 2H), 1.02 (m, 2H), 1.89 (s, 3H), 1.98 (m, 1H), 2.42 (s, 3H), 7.22 (s, 1H), 7.46 (t, 1H), 7.52 (d, 1H), 7.72 (d, 1H), 7.75 (s, 1H), 8.36 (s, 1H), 11.37 (s, 1H), 13.29 (s, 1H).

### Example 5: Preparation of 3-[5-(3-Chloropyridin-4-Yl)-2-[(5-Cyclopropyl-1H-1,2,4-Triazol-3-Yl)Amino]-1,3-Thiazol-4-Yl]Benzonitrile (Compound 5)

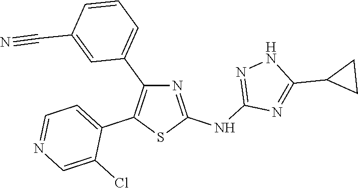 [SMILES: N#Cc1cccc(-c2nc(Nc3n[nH]c(C4CC4)n3)sc2-c2ccncc2Cl)c1]

Compound 5 was prepared from 3-[(3-chloropyridin-4-yl)acetyl]benzonitrile (Intermediate IV-5), Br₂, and N-(5-cyclopropyl-1H-1,2,4-triazol-3-yl)thiourea (Intermediate III-2) according to Preparative Example 26. Scale: 1.8 mmol; 0.32 g; 43% yield. [M+H]⁺: 420.0. ¹H NMR (400 MHz, DMSO-d₆): δ (ppm) 0.91 (m, 2H), 1.04 (m, 2H), 2.00 (m, 1H), 7.53 (m, 3H), 7.76 (s, 2H), 8.53 (s, 1H), 8.73 (s, 1H), 11.55 (s, 1H), 13.35 (s, 1H).

### Example 6: Preparation of 3-{2-[(5-Cyclopropyl-1H-1,2,4-Triazol-3-Yl)Amino]-5-(2,3-Dimethylpyridin-4-Yl)-1,3-Thiazol-4-Yl}Benzonitrile (Compound 6)

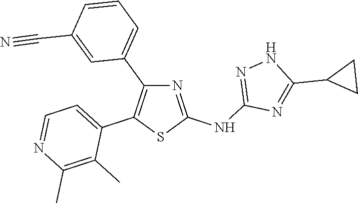 [SMILES: Cc1nccc(-c2sc(Nc3n[nH]c(C4CC4)n3)nc2-c2cccc(C#N)c2)c1C]

Compound 6 was prepared from 3-[(2,3-dimethylpyridin-4-yl)acetyl]benzonitrile (Intermediate IV-6), Br₂, and N-(5-cyclopropyl-1H-1,2,4-triazol-3-yl)thiourea (Intermediate III-2) according to Preparative Example 27. Scale: 0.22 mmol; 0.023 g; 25% yield. [M+H]⁺: 414.2. ¹H NMR (500 MHz, DMSO-d₆): δ (ppm) 0.92 (m, 2H), 1.04 (m, 2H), 2.00 (s, 3H), 2.02 (m, 1H), 2.48 (s, 3H), 7.18 (d, 1H), 7.48 (t, 1H), 7.54 (d, 1H), 7.73 (d, 1H), 7.78 (s, 1H), 8.32 (d, 1H), 11.37 (s, 1H), 13.29 (s, 1H).

### Example 7: Preparation of 3-{2-[(5-Cyclopropyl-1H-1,2,4-Triazol-3-Yl)Amino]-5-(2-Methylpyridin-4-Yl)-1,3-Thiazol-4-Yl}Benzonitrile (Compound 7)

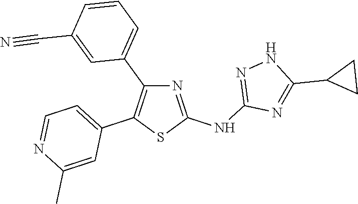 [SMILES: Cc1cc(-c2sc(Nc3n[nH]c(C4CC4)n3)nc2-c2cccc(C#N)c2)ccn1]

Compound 7 was prepared from 3-[(2-methylpyridin-4-yl)acetyl]benzonitrile (Intermediate IV-7), Br₂, and N-(5-cyclopropyl-1H-1,2,4-triazol-3-yl)thiourea (Intermediate III-2) according to Preparative Example 27. Scale: 0.45 mmol; 0.027 g; 15% yield. [M+H]⁺: 400.0. ¹H NMR (500 MHz, DMSO-d₆): δ (ppm) 0.92 (m, 2H), 1.05 (m, 2H), 2.02 (m, 1H), 2.54 (s, 3H), 7.22 (d, 1H), 7.55 (s, 1H), 7.61 (t, 1H), 7.77 (d, 1H), 7.89 (d, 1H), 7.92 (s, 1H), 8.42 (d, 1H), 11.82 (s, 1H), 13.41 (s, 1H).

### Example 8: Preparation of 3-{2-[(5-Cyclopropyl-1H-1,2,4-Triazol-3-Yl)Amino]-5-(2-Methyl-1H-Imidazol-1-Yl)-1,3-Thiazol-4-Yl}Benzonitrile (Compound 8)

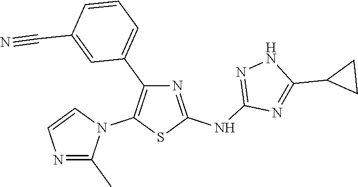 [SMILES: Cc1nccn1-c1sc(Nc2n[nH]c(C3CC3)n2)nc1-c1cccc(C#N)c1]

Compound 8 was prepared from 3-[(2-methyl-1H-imidazol-1-yl)acetyl]benzonitrile (Intermediate IV-8), I₂, and N-(5-cyclopropyl-1H-1,2,4-triazol-3-yl)thiourea (Intermediate III-2) according to Preparative Example 28. Scale: 0.77 mmol.

Specifically, A mixture of 3-[(2-methyl-1H-imidazol-1-yl)acetyl]benzonitrile (0.174 g, 0.77 mmol), N-(5-cyclopropyl-1H-1,2,4-triazol-3-yl)thiourea (0.142 g, 0.77 mmol), iodine (0.196 g, 0.77 mmol) and pyridine (2 mL) was stirred at r.t. overnight. The resulting mixture was diluted with water (50 mL). The solid obtained was filtered, stirred in water (50 mL) for 30 minutes, and filtered again. The crude product was purified by preparative HPLC to afford Compound 8 (0.022 g, 7.3% yield). [M+H]⁺: 389.0. ¹H NMR (500 MHz, DMSO-d₆): δ (ppm) 0.90 (m, 2H), 1.03 (m, 2H), 1.96 (m, 1H), 2.01 (s, 3H), 7.03 (s, 1H), 7.35 (s, 1H), 7.39 (d, 1H), 7.48 (s, 1H), 7.54 (t, 1H), 7.76 (d, 1H), 11.57 (s, 1H), 13.37 (s, 1H).

### Example 9: Preparation of 3-[5-(2-Methyl-1H-Imidazol-1-Yl)-2-[(1H-1,2,4-Triazol-3-Yl)Amino]-1,3-Thiazol-4-Yl]Benzonitrile (Compound 9)

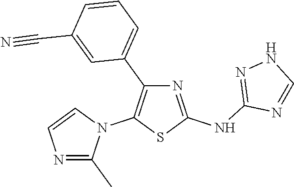 [SMILES: Cc1nccn1-c1sc(Nc2nc[nH]n2)nc1-c1cccc(C#N)c1]

Compound 9 was prepared from 3-[(2-methyl-1H-imidazol-1-yl)acetyl]benzonitrile (Intermediate IV-8), I₂, and N-1H-1,2,4-triazol-3-ylthiourea (Intermediate III-3) according to Preparative Example 28. Scale: 0.57 mmol; 0.047 g 23% yield. [M+H]⁺: 349.2. ¹H NMR (DMSO-d₆, 400 MHz): δ (ppm) 2.04 (s, 3H), 7.05 (s, 1H), 7.36 (s, 1H), 7.42 (d, 1H), 7.48-7.62 (m, 2H), 7.79 (d, 1H), 8.48 (s, 1H), 11.76 (s, 1H), 13.79 (s, 1H).

### Example 10: Preparation of 3-{2-[(5-Methyl-1H-1,2,4-Triazol-3-Yl)Amino]-5-(2-Methyl-1H-Imidazol-1-Yl)-1,3-Thiazol-4-Yl}Benzonitrile (Compound 10)

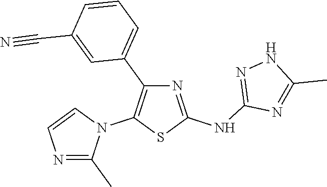 [SMILES: Cc1nc(Nc2nc(-c3cccc(C#N)c3)c(-n3ccnc3C)s2)n[nH]1]

Compound 10 was prepared from 3-[(2-methyl-1H-imidazol-1-yl)acetyl]benzonitrile (Intermediate IV-8), I₂, and N-(5-methyl-1H-1,2,4-triazol-3-yl)thiourea (Intermediate III-4) according to Preparative Example 28. Scale: 0.55 mmol; 0.046 g 23% yield. [M+H]⁺: 363.2. ¹H NMR (DMSO-d₆ 400 MHz): δ (ppm) 2.03 (s, 3H), 2.34 (s, 3H), 7.05 (s, 1H), 7.36 (s, 1H), 7.42 (d, 1H), 7.51 (s, 1H), 7.56 (t, 1H), 7.79 (d, 1H), 11.62 (s, 1H), 13.35 (s, 1H).

### Example 11: Preparation of 3-(2-{[5-(Hydroxymethyl)-1H-1,2,4-Triazol-3-Yl]Amino}-5-(2-Methyl-1H-Imidazol-1-Yl)-1,3-Thiazol-4-Yl)Benzonitrile (Compound 11)

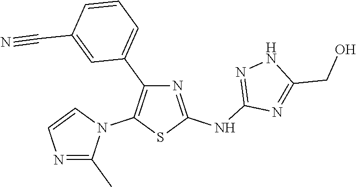 [SMILES: Cc1nccn1-c1sc(Nc2n[nH]c(CO)n2)nc1-c1cccc(C#N)c1]

Compound 11 was prepared from 3-[(2-methyl-1H-imidazol-1-yl)acetyl]benzonitrile (Intermediate IV-8), I₂, and N-[5-(hydroxymethyl)-1H-1,2,4-triazol-3-yl]thiourea (Intermediate III-5) according to Preparative Example 28. Scale: 0.55 mmol; 0.069 g 34% yield. [M+H]⁺: 379.0. ¹H NMR (DMSO-d₆, 400 MHz): δ (ppm) 2.04 (s, 3H), 4.56 (d, 2H), 5.72 (s, 1H), 7.05 (d, 1H), 7.36 (d, 1H), 7.42 (d, 1H), 7.49-7.60 (m, 2H), 7.78 (d, 1H), 11.67 (s, 1H), 13.61 (s, 1H).

### Example 12: Preparation of 3-[5-(2,5-Dimethylpyridin-4-Yl)-2-[(1H-1,2,4-Triazol-3-Yl)Amino]-1,3-Thiazol-4-Yl]Benzonitrile (Compound 12)

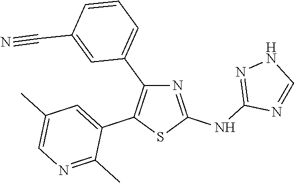 [SMILES: Cc1cnc(C)c(-c2sc(Nc3nc[nH]n3)nc2-c2cccc(C#N)c2)c1]

Compound 12 was prepared from 3-[(2,5-dimethylpyridin-4-yl)acetyl]benzonitrile (Intermediate IV-4), Br₂, and N-1H-1,2,4-triazol-3-ylthiourea (Intermediate III-3) according to Preparative Example 27. Scale 0.27 mmol; 0.039 g; 39% yield. [M+H]⁺: 374.4. ¹H NMR (DMSO-d₆, 400 MHz): δ (ppm) 1.93 (s, 3H), 2.44 (s, 3H), 7.24 (s, 1H), 7.49 (t, 1H), 7.55 (d, 1H), 7.74 (d, 1H), 7.81 (s, 1H), 8.19-8.57 (m, 2H), 11.37-12.19 (m, 1H), 13.66 (s, 1H).

### Example 13: Preparation of 3-[5-(2,5-Dimethylpyridin-4-Yl)-2-[(5-Methyl-1H-1,2,4-Triazol-3-Yl)Amino]-1,3-Thiazol-4-Yl]Benzonitrile (Compound 13)

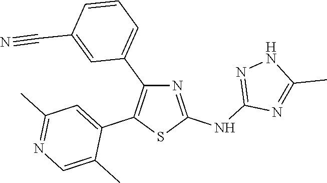 [SMILES: Cc1cc(-c2sc(Nc3n[nH]c(C)n3)nc2-c2cccc(C#N)c2)c(C)cn1]

Compound 13 was prepared from 3-[(2,5-dimethylpyridin-4-yl)acetyl]benzonitrile (Intermediate IV-4), Br₂, and N-(5-methyl-1H-1,2,4-triazol-3-yl)thiourea (Intermediate III-4) according to Preparative Example 27. Scale: 0.26 mmol; 0.013 g; 13% yield. [M+H]⁺: 388.2. ¹H NMR (DMSO-d₆, 400 MHz): δ (ppm) 1.91 (s, 3H), 2.32 (s, 3H), 2.44 (s, 3H), 7.24 (s, 1H), 7.49 (t, 1H), 7.55 (d, 1H), 7.74 (d, 1H), 7.79 (s, 1H), 8.37 (s, 1H), 11.41 (s, 1H), 13.24 (s, 1H).

### Example 14: Preparation of 3-[5-(2,5-Dimethylpyridin-4-Yl)-2-{[5-(Hydroxymethyl)-1H-1,2,4-Triazol-3-Yl]Amino}-1,3-Thiazol-4-Yl]Benzonitrile (Compound 14)

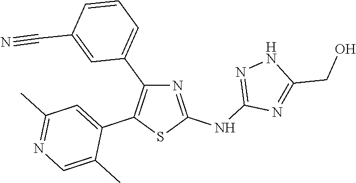 [SMILES: Cc1cc(-c2sc(Nc3n[nH]c(CO)n3)nc2-c2cccc(C#N)c2)c(C)cn1]

Compound 14 was prepared from 3-[(2,5-dimethylpyridin-4-yl)acetyl]benzonitrile (Intermediate IV-4), Br₂, and N-[5-(hydroxymethyl)-1H-1,2,4-triazol-3-yl]thiourea (Intermediate III-5) according to Preparative Example 27. Scale: 0.25 mmol; 0.041 g; 41% yield. [M+H]⁺: 404.2. ¹H NMR (DMSO-d₆, 400 MHz): δ (ppm) 1.91 (s, 3H), 2.44 (s, 3H), 4.55 (d, 2H), 5.57-5.75 (m, 1H), 7.25 (s, 1H), 7.49 (t, 1H), 7.55 (d, 1H), 7.74 (d, 1H), 7.80 (s, 1H), 8.37 (s, 1H), 11.58 (s, 1H), 13.39 (s, 1H).

### Example 15: Preparation of 3-{2-[(5-Cyclopropyl-1H-1,2,4-Triazol-3-Yl)Amino]-5-(1H-1,2,4-Triazol-1-Yl)-1,3-Thiazol-4-Yl}Benzonitrile (Compound 15)

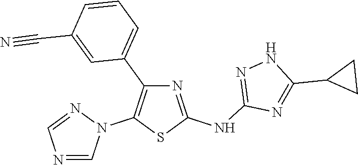 [SMILES: N#Cc1cccc(-c2nc(Nc3n[nH]c(C4CC4)n3)sc2-n2cncn2)c1]

Compound 15 was prepared from 3-[(1H-1,2,4-triazol-1-yl)acetyl]benzonitrile (Intermediate IV-9), I₂, and N-(5-cyclopropyl-1H-1,2,4-triazol-3-yl)thiourea (Intermediate III-2) according to Preparative Example 28. Scale: 0.4 mmol; 0.013 g, 9% yield. [M+H]⁺: 376.2. ¹H NMR (DMSO-d₆, 400 MHz): δ (ppm) 0.89-0.96 (m, 2H), 1.03-1.07 (m, 2H), 1.95-2.05 (m, 1H), 7.46 (d, 1H), 7.54-7.61 (m, 2H), 7.81 (d, 1H), 8.37 (s, 1H), 8.86 (s, 1H), 11.73 (s, 1H), 13.44 (s, 1H).

### Example 16: Preparation of 3-[5-(2-Ethyl-1H-Imidazol-1-Yl)-2-[(5-Methyl-1H-1,2,4-Triazol-3-Yl)Amino]-1,3-Thiazol-4-Yl]Benzonitrile (Compound 16)

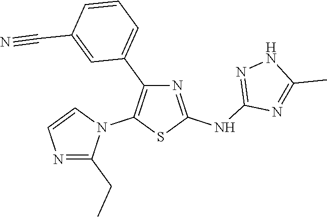 [SMILES: CCc1nccn1-c1sc(Nc2n[nH]c(C)n2)nc1-c1cccc(C#N)c1]

Compound 16 was prepared from 3-[(2-ethyl-1H-imidazol-1-yl)acetyl]benzonitrile (Intermediate IV-10), I₂, and N-(5-methyl-1H-1,2,4-triazol-3-yl)thiourea (Intermediate III-4) according to Preparative Example 28. Scale: 0.53 mmol; 0.038 g, 19% yield. [M+H]⁺: 377.0. ¹H NMR (DMSO-d₆, 400 MHz): δ (ppm) 0.99 (t, 3H), 2.29-2.38 (m, 5H), 7.08 (d, 1H), 7.37 (d, 1H), 7.40 (d, 1H), 7.49-7.59 (m, 2H), 7.78 (d, 1H), 11.59 (s, 1H), 13.33 (s, 1H).

### Example 17: Preparation of 3-{2-[(5-Cyclopropyl-1H-1,2,4-Triazol-3-Yl)Amino]-5-[2-(Hydroxymethyl)-1H-Imidazol-1-Yl]-1,3-Thiazol-4-Yl}Benzonitrile (Compound 17)

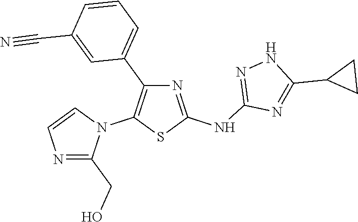 [SMILES: N#Cc1cccc(-c2nc(Nc3n[nH]c(C4CC4)n3)sc2-n2ccnc2CO)c1]

Compound 17 was prepared from 3-{[2-(hydroxymethyl)-1H-imidazol-1-yl]acetyl}benzonitrile (Intermediate IV-11), I₂, and N-(5-cyclopropyl-1H-1,2,4-triazol-3-yl)thiourea (Intermediate III-2) according to Preparative Example 28. Scale: 0.5 mmol; 0.041 g, 20% yield. [M+H]⁺: 405.2. ¹H NMR (DMSO-d₆, 400 MHz): δ (ppm) 0.83-0.95 (m, 2H), 0.96-1.10 (m, 2H), 1.94-2.04 (m, 1H), 4.28 (d, 2H), 5.28 (t, 1H), 7.09 (s, 1H), 7.32 (s, 1H), 7.44-7.56 (m, 3H), 7.75 (d, 1H), 11.53 (s, 1H), 13.37 (s, 1H).

### Example 18: Preparation of 3-{2-[(5-Cyclopropyl-1H-1,2,4-Triazol-3-Yl)Amino]-5-(2-Methyl-1H-Imidazol-1-Yl)-1,3-Thiazol-4-Yl}-4-Fluorobenzonitrile (Compound 18)

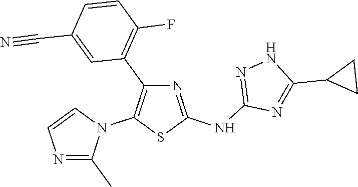 [SMILES: Cc1nccn1-c1sc(Nc2n[nH]c(C3CC3)n2)nc1-c1cc(C#N)ccc1F]

Compound 18 was prepared from 4-fluoro-3-[(2-methyl-1H-imidazol-1-yl)acetyl]benzonitrile (Intermediate IV-12), I₂, and N-(5-cyclopropyl-1H-1,2,4-triazol-3-yl)thiourea (Intermediate III-2) according to Preparative Example 28. Scale: 1.1 mmol; 0.022 g, 7.3% yield. [M+H]⁺: 407.0. ¹H NMR (DMSO-d₆, 400 MHz): δ (ppm) 0.88-0.94 (m, 2H), 1.01-1.07 (m, 2H), 1.95-2.03 (m, 1H), 2.05 (s, 3H), 6.84 (d, 1H), 7.17 (d, 1H), 7.46 (t, 1H), 7.91-7.97 (m, 2H), 11.59 (s, 1H), 13.39 (s, 1H).

### Example 19: Preparation of 3-{5-[2-(Hydroxymethyl)-1H-Imidazol-1-Yl]-2-[(1H-1,2,4-Triazol-3-Yl)Amino]-1,3-Thiazol-4-Yl}Benzonitrile (Compound 19)

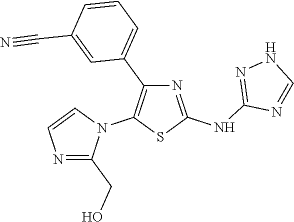 [SMILES: N#Cc1cccc(-c2nc(Nc3nc[nH]n3)sc2-n2ccnc2CO)c1]

Compound 19 was prepared from 3-{[2-(hydroxymethyl)-1H-imidazol-1-yl]acetyl}benzonitrile (Intermediate IV-11), I₂, and N-1H-1,2,4-triazol-3-ylthiourea (Intermediate III-3) according to Preparative Example 28. Scale: 0.41 mmol; 0.025 g, 17% yield. [M+H]⁺: 365.08. ¹H NMR (DMSO-d₆, 500 MHz): δ (ppm) 2.29 (s, 3H), 4.26 (d, J=5.1 Hz, 2H), 5.24 (t, J=5.3, 5.3 Hz, 1H), 7.06 (s, 1H), 7.27 (s, 1H), 7.42-7.55 (m, 3H), 7.72 (d, J=7.4 Hz, 1H), 11.61 (s, 1H), 13.21 (s, 1H).

### Example 20: Preparation of 3-{2-[(5-Ethyl-1H-1,2,4-Triazol-3-Yl)Amino]-5-[2-(Hydroxymethyl)-1H-Imidazol-1-Yl]-1,3-Thiazol-4-Yl}Benzonitrile (Compound 20)

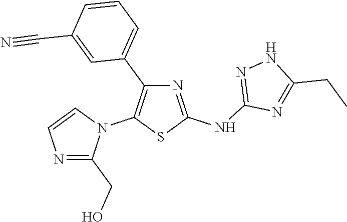 [SMILES: CCc1nc(Nc2nc(-c3cccc(C#N)c3)c(-n3ccnc3CO)s2)n[nH]1]

Compound 20 was prepared from 3-{[2-(hydroxymethyl)-1H-imidazol-1-yl]acetyl}benzonitrile (Intermediate IV-11), I₂, and N-(5-ethyl-1H-1,2,4-triazol-3-yl)thiourea (Intermediate III-6) according to Preparative Example 28. Scale: 0.51 mmol; 0.045 g, 22% yield. [M+H]⁺: 393.12. ¹H NMR (DMSO-d₆, 500 MHz): δ (ppm) 1.22 (t, J=7.6, 7.6 Hz, 3H), 2.67 (q, J=7.6, 7.6, 7.6 Hz, 2H), 4.27 (d, J=5.4 Hz, 2H), 5.26 (t, J=5.6, 5.6 Hz, 1H), 7.07 (d, J=1.4 Hz, 1H), 7.30 (d, J=1.4 Hz, 1H), 7.46 (dt, J=8.1, 1.5, 1.5 Hz, 1H), 7.49-7.55 (m, 2H), 7.73 (dt, J=7.5, 1.5, 1.5 Hz, 1H), 11.55 (s, 1H), 13.32 (s, 1H).

### Example 21: Preparation of 3-{5-[2-(Hydroxymethyl)-1H-Imidazol-1-Yl]-2-[(5-Methyl-1H-1,2,4-Triazol-3-Yl)Amino]-1,3-Thiazol-4-Yl}Benzonitrile (Compound 21)

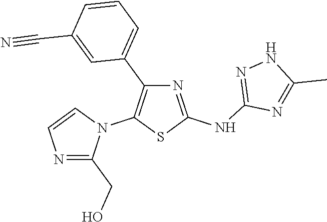 [SMILES: Cc1nc(Nc2nc(-c3cccc(C#N)c3)c(-n3ccnc3CO)s2)n[nH]1]

Compound 21 was prepared from 3-{[2-(hydroxymethyl)-1H-imidazol-1-yl]acetyl}benzonitrile (Intermediate IV-11), I₂, and N-(5-methyl-1H-1,2,4-triazol-3-yl)thiourea (Intermediate III-4) according to Preparative Example 28. Scale: 0.4 mmol; 0.015 g, 10% yield. [M+H]⁺: 379.1. ¹H NMR (DMSO-d₆, 500 MHz): δ (ppm) 2.29 (s, 3H), 4.26 (d, J=5.1 Hz, 2H), 5.24 (t, J=5.3, 5.3 Hz, 1H), 7.06 (s, 1H), 7.27 (s, 1H), 7.42-7.55 (m, 3H), 7.72 (d, J=7.4 Hz, 1H), 11.61 (s, 1H), 13.21 (s, 1H).

### Example 22: Preparation of 3-{5-(3-Methylpyridin-4-Yl)-2-[(1H-1,2,4-Triazol-3-Yl)Amino]-1,3-Thiazol-4-Yl}Benzonitrile (Compound 22)

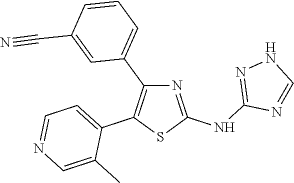 [SMILES: Cc1cnccc1-c1sc(Nc2nc[nH]n2)nc1-c1cccc(C#N)c1]

Compound 22 was prepared from 3-[(3-methylpyridin-4-yl)acetyl]benzonitrile (intermediate IV-3) Br₂, and N-1H-1,2,4-triazol-3-ylthiourea (intermediate III-3) according to Preparative Example 27. Scale: 0.84 mmol; 0.035 g, 12% yield. [M+H]⁺: 360.1. ¹H NMR (DMSO-d₆, 400 MHz): δ (ppm) 2.03 (s, 3H), 7.33 (d, 1H), 7.49 (t, 1H), 7.55 (m, 1H), 7.74 (d, 1H), 7.78 (s, 1H), 8.38 (s, 1H), 8.45 (d, 1H), 8.53 (s, 1H), 11.69 (s, 1H), 13.66 (s, 1H).

### Example 23: Preparation of 3-{2-[(5-Cyclopropyl-1H-1,2,4-Triazol-3-Yl)Amino]-5-(5-Methylpyridazin-4-Yl)-1,3-Thiazol-4-Yl}Benzonitrile (Compound 23)

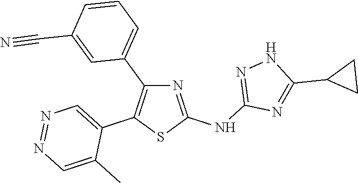 [SMILES: Cc1cnncc1-c1sc(Nc2n[nH]c(C3CC3)n2)nc1-c1cccc(C#N)c1]

Compound 23 was prepared from 3-[(5-methylpyridazin-4-yl)acetyl]benzonitrile (Intermediate IV-13)), Br₂, and N-(5-cyclopropyl-1H-1,2,4-triazol-3-yl)thiourea (Intermediate III-2) according to Preparative Example 27. Scale: 0.62 mmol; 0.058 g, 23% yield. [M+H]⁺: 401.13 ¹H NMR (DMSO-d₆, 400 MHz): δ (ppm) 0.91 (m, 2H), 1.03 (m, 2H), 2.00 (tt, 1H), 2.10 (s, 3H), 7.51 (m, 2H), 7.78 (m, 2H), 8.95 (s, 1H), 9.16 (s, 1H), 11.59 (s, 1H), 13.34 (s, 1H).

### Example 24: Preparation of 3-{2-[(5-Cyclopropyl-1H-1,2,4-Triazol-3-Yl)Amino]-5-(Pyrimidin-4-Yl)-1,3-Thiazol-4-Yl}Benzonitrile (Compound 24)

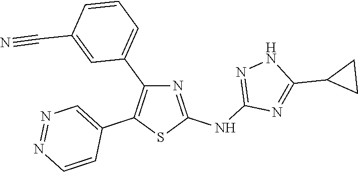 [SMILES: N#Cc1cccc(-c2nc(Nc3n[nH]c(C4CC4)n3)sc2-c2ccnnc2)c1]

Compound 24 was prepared from 3-[(pyrimidin-4-yl)acetyl]benzonitrile (Intermediate IV-14)), Br₂, and N-(5-cyclopropyl-1H-1,2,4-triazol-3-yl)thiourea (Intermediate III-2) according to Preparative Example 27. Scale: 0.78 mmol; 0.045 g, 15% yield. [M+H]⁺: 387.1 ¹H NMR (DMSO-d₆, 400 MHz): δ (ppm) 0.94 (m, 2H), 1.05 (m, 2H), 2.04 (tt, 1H), 6.97 (dd, 1H), 7.68 (t, 1H), 7.88 (d, 1H), 7.95 (d, 1H), 8.02 (s, 1H), 8.49 (d, 1H), 9.04 (d, 1H), 11.63 (s, 1H), 13.37 (s, 1H).

### Example 25: Preparation of 3-{2-[(5-Cyclopropyl-1H-1,2,4-Triazol-3-Yl)Amino]-5-(Pyridazin-4-Yl)-1,3-Thiazol-4-Yl}Benzonitrile (Compound 25)

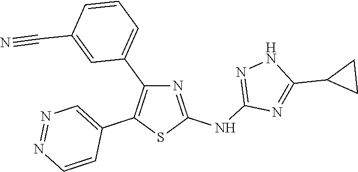 [SMILES: N#Cc1cccc(-c2nc(Nc3n[nH]c(C4CC4)n3)sc2-c2ccnnc2)c1]

Compound 25 was prepared from 3-[(pyridazin-4-yl)acetyl]benzonitrile (Intermediate IV-15), Br₂, and N-(5-cyclopropyl-1H-1,2,4-triazol-3-yl)thiourea (Intermediate III-2) according to Preparative Example 27. Scale: 0.78 mmol; 0.039 g, 13% yield. [M+H]⁺: 387.11 ¹H NMR (DMSO-d₆, 400 MHz): δ (ppm) 0.94 (m, 2H), 1.05 (m, 2H), 2.02 (m, 1H), 7.51 (dd, 1H), 7.60 (t, 1H), 7.74 (d, 1H), 7.87 (d, 1H), 7.91 (s, 1H), 8.97 (s, 1H), 9.11 (d, 1H), 11.61 (s, 1H), 13.33 (s, 1H).

### Example 26: Preparation of 3-{2-[(5-Cyclopropyl-1H-1,2,4-Triazol-3-Yl)Amino]-5-(Pyridin-4-Yl)-1,3-Thiazol-4-Yl}Benzonitrile (Compound 26)

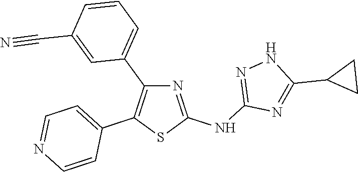 [SMILES: N#Cc1cccc(-c2nc(Nc3n[nH]c(C4CC4)n3)sc2-c2ccncc2)c1]

Compound 26 was prepared from 3-[(pyridin-4-yl)acetyl]benzonitrile (Intermediate IV-16), Br₂, and N-(5-cyclopropyl-1H-1,2,4-triazol-3-yl)thiourea (Intermediate III-2) according to Preparative Example 27. Scale: 0.9 mmol; 0.075 g, 25% yield. [M+H]⁺: 386.12 ¹H NMR (DMSO-d₆, 400 MHz): δ (ppm) 0.92 (m, 2H), 1.04 (m, 2H), 2.00 (m, 1H), 7.22 (m, 2H), 7.57 (t, 1H), 7.71 (d, 1H), 7.83 (d, 1H), 7.87 (s, 1H), 8.49 (m, 2H), 11.53 (s, 1H), 13.33 (s, 1H).

### Example 27: Preparation of 3-{2-[(5-Cyclopropyl-1H-1,2,4-Triazol-3-Yl)Amino]-5-(5-Methylpyrimidin-4-Yl)-1,3-Thiazol-4-Yl}Benzonitrile (Compound 27)

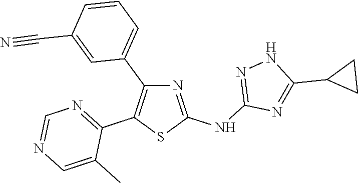 [SMILES: Cc1cncnc1-c1sc(Nc2n[nH]c(C3CC3)n2)nc1-c1cccc(C#N)c1]

Compound 27 was prepared from 3-[(5-methylpyrimidin-4-yl)acetyl]benzonitrile (Intermediate IV-17), Br₂, and N-(5-cyclopropyl-1H-1,2,4-triazol-3-yl)thiourea (Intermediate III-2) according to Preparative Example 27. Scale: 0.62 mmol; 0.033 g, 13% yield. [M+H]⁺: 401.13. 1H NMR (DMSO-d₆, 400 MHz): δ (ppm) 0.91 (m, 2H), 1.03 (m, 2H), 1.82 (s, 3H), 2.00 (tt, 1H), 7.53 (m, 2H), 7.80 (m, 2H), 8.66 (s, 1H), 9.05 (s, 1H), 11.56 (s, 1H), 13.33 (s, 1H).

### Example 28: Preparation of 3-{2-[(5-Cyclopropyl-1H-1,2,4-Triazol-3-Yl)Amino]-5-(2-Ethyl-1H-Imidazol-1-Yl)-1,3-Thiazol-4-Yl}Benzonitrile (Compound 28)

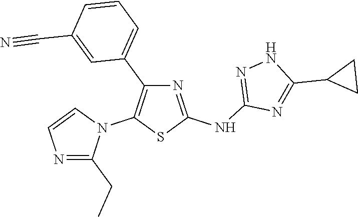 [SMILES: CCc1nccn1-c1sc(Nc2n[nH]c(C3CC3)n2)nc1-c1cccc(C#N)c1]

Compound 28 was prepared from 3-[(2-ethyl-1H-imidazol-1-yl)acetyl]benzonitrile (Intermediate IV-10), I₂, and N-(5-cyclopropyl-1H-1,2,4-triazol-3-yl)thiourea (Intermediate III-2) according to Preparative Example 28. Scale: 0.4 mmol; 0.015 g, 10% yield. [M+H]⁺: 403.2. ¹H NMR (DMSO-d₆, 400 MHz): δ (ppm) 0.92 (m, 2H), 0.99 (t, 3H), 1.04 (m, 2H), 2.00 (m, 1H), 2.36 (q, 2H), 7.10 (s, 1H), 7.39 (m, 2H), 7.51 (s, 1H), 7.56 (t, 1H), 7.78 (d, 1H), 11.59 (s, 1H), 13.39 (s, 1H).

### Example 29: Preparation of 3-{2-[(5-Cyclopropyl-1H-1,2,4-Triazol-3-Yl)Amino]-5-(1-Methyl-6-Oxo-1,6-Dihydropyridin-3-Yl)-1,3-Thiazol-4-Yl}Benzonitrile (Compound 29)

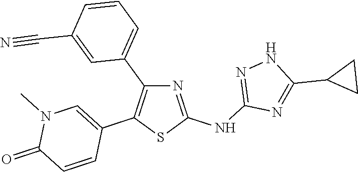 [SMILES: Cn1cc(-c2sc(Nc3n[nH]c(C4CC4)n3)nc2-c2cccc(C#N)c2)ccc1=O]

Compound 29 was prepared from of 3-[(1-methyl-6-oxo-1,6-dihydropyridin-3-yl)acetyl]benzonitrile (Intermediate IV-18), I₂, and N-(5-cyclopropyl-1H-1,2,4-triazol-3-yl)thiourea (Intermediate III-2) according to Preparative Example 28. Scale: 0.24 mmol; 0.017 g, 17% yield. [M+H]⁺: 416.2. ¹H NMR (DMSO-d₆, 500 MHz): δ (ppm) 0.89 (m, 2H), 1.01 (m, 2H), 1.97 (m, 1H), 3.43 (s, 3H), 6.35 (d, 1H), 7.17 (dd, 1H), 7.53 (t, 1H), 7.74 (d, 1H), 7.77 (m, 1H), 7.90 (s, 2H), 11.23 (s, 1H), 13.23 (s, 1H).

### Example 30: Intracellular Camp Functional Assay of Compounds of Formula I

Compound activity in human adenosine receptors A_{2A}, A_{2B}, A₁, and A₃ was measured in this functional cellular assay using an HTRF kit (Perkin Elmer). Prior to the test, HEK293 cells expressing recombinant human adenosine receptors were grown in media without antibiotic, were detached by gentle flushing with PBS-EDTA (5 mM EDTA), were recovered by centrifugation and were resuspended in assay buffer (KRH: 5 mM KCl, 1.25 mM MgSO₄, 124 mM NaCl, 25 mM HEPES, 13.3 mM Glucose, 1.25 mM KH₂PO₄, 1.45 mM CaCl₂, 0.5 g/l BSA, supplemented with 1 mM IBMX or 25 μM Rolipram). Dose response curves were performed in parallel with the reference compounds. The reference compounds used for A_{2A}, A_{2B}, A₁, and A₃, were respectively NECA, ZM241385, DPCPX, MRS. 12 μl of cells were mixed with 6 μl of the test compound at increasing concentrations and then incubated 10 min. Thereafter 6 μl of the reference agonist was added at a final concentration corresponding to the historical EC₈₀. The plates were then incubated for 30 min at room temperature. After addition of the lysis buffer and 1 hour incubation, cAMP concentrations were estimated, according to the manufacturer specification, with the HTRF kit. The results showing functional activity of embodied compounds on adenosine receptors are shown below in Table 1.

### Example 31: Radioligand Binding Assay

A filtration binding assay was performed for adenosine receptors A₁, A_{2A}, A_{2B} and A₃. Radioligand binding competition assay was done in duplicate in the wells of a 96-well plate (Master Block, Greiner, 786201) containing binding buffer, receptor membrane extracts, a fixed concentration of tracer, and test compound at increasing concentrations. In order to eliminate effect of buffer components, binding buffer was the same for all four receptors and contained: 50 mM Tris-HCl pH 7.4, 5 mM MgCl, 1 mM EDTA, 150 mM NaCl, 0.1% Na-azide, and 5 U/ml adenosine-deaminase. Nonspecific binding was minimized by co-incubation with 200-fold excess of cold competitor. In all radioligand binding experiments, the samples were incubated in a final volume of 0.1 ml of binding buffer for 60 minutes at 25° C. and then filtered over Unifilter plates (Perkin Elmer) pre-treated for 2 hours to limit tracer non-specific binding. Filters were washed five times with 0.5 ml of ice-cold washing buffer (50 mM Tris-HCl pH 7.4, 5 mM MgCl, 1 mM EDTA), and 50 μL of Microscint 20 (Perkin Elmer) were added to each filter. The plates were incubated 15 min at room temperature on an orbital shaker and then counted with a TopCount™ for 1 min/well.

For A₁ receptor, the assay was performed with [3H]-DPCPX and membranes from CHO-Kl cells transfected with human A₁ receptor (Euroscreen FAST-001B).

For A_{2A} receptor, the assay was performed with [3H-NECA and membranes from HEK293 cells transfected with human A_{2A} receptor (Euroscreen FAST-002B).

For A_{2B} receptor, the assay was carried with [3H]-DPCPX and membranes prepared from HEK293 cells transfected with human A_{2B} receptor (Euroscreen FAST-003B).

For A₃ receptor, the assay was carried out with [125IJ-MECA and membranes from CHO-Kl cells transfected with human A₃ receptor (Euroscreen FAST-004B).

Table 1 shows the in vitro cAMP binding activity of embodied compounds as measured by the functional cAMP accumulation assay in HEK293 cells (Example 30), and the radioligand binding assay (Example 31). Table 1 shows human A_{2B} affinity of embodied compounds versus binding affinities for human A₁, and A₃ receptors. The results demonstrate high selectivity for A_{2B} (and A₁) receptors over A₃ receptors.

TABLE 1
Binding Affinities of Selected Compounds as measured
by Intracellular Assay and Radioligand Binding

| | Human A_{2B} | Human A₁ | Human A₃ | Human A_{2A} |
|---|---|---|---|---|
| Cmpd. | EC₅₀ (nM) | EC₅₀ (nM) | EC₅₀ (nM) | EC₅₀ (nM) |
| 1 | 2.2* | <5.0* | >10000* | >10000* |
| 2 | 0.30 | 0.78 | >20000 | 78.9 |
| 3 | 0.38 | 1.8 | >20000 | 119 |
| 4 | 0.99 | 16.4 | >20000 | 3888 |
| 5 | 0.76 | 2.3 | 2345 | 478 |
| 6 | 0.84 | 1.2 | 2063 | 206 |
| 7 | 1.4 | 1.7 | 1737 | 24.4 |
| 8 | 2.9 | 107 | >20000 | >10000 |
| 9 | 3.3 | 145 | 2345 | >10000 |
| 12 | 1.5 | 251 | >20000 | >10000 |
| 13 | 2.8 | 65 | >20000 | >10000 |
| 14 | 3.0 | 36.1 | >20000 | >10000 |
| 16 | 2.2 | — | — | 61 |
| 17 | 2.4 | — | — | 100 |
| 18 | 33.4 | — | — | >10000 |
| 19 | 2.01 | 885 | >3.40 × 10³ | 5760 |
| 20 | 1.26 | 360 | >30 × 10³ | 3410 |
| 21 | 1.64 | >30 × 10³ | >30 × 10³ | 6980 |
| 22 | 0.6 | 2.3 | >30 × 10³ | — |
| 23 | 6.7 | 115 | >30 × 10³ | — |
| 24 | 2.3 | 24.1 | >30 × 10³ | — |
| 25 | 2.4 | 2.6 | >30 × 10³ | — |
| 26 | 0.7 | 0.8 | >30 × 10³ | — |
| 27 | 1.7 | 20.7 | >30 × 10³ | — |
| 29 | 0.4 | 4.8 | >30 × 10³ | 1.1 |

*Measured by radioligand binding.

### Example 32: CPM Thermostability Assay

Compounds of the present invention were screened for A_{2B}/A_{2A} receptor binding and stabilization using CPM thermostability assay (A. I. Alexandrov et al., 2008, Structure 16, 351-359). Protein concentration in the reaction was 1 μM, while the compound concentration for the data shown was 10 μM. Final concentration of the CPM dye (Invitrogen D346) in the reaction was 7.5 μg/ml. The reaction buffer for A_{2B} was 40 mM Tris-HCl pH 7.4, 200 mM NaCl, 0.05% DDM, and 0.005% CHS. The reaction buffer for A_{2A} was 40 mM Tris-HCl pH 7.4, 200 mM NaCl, and 0.15% DDM. To allow for binding, compounds were incubated with the purified receptor for 30 minutes on ice before addition of the CPM dye. Measurements were performed using Rotor-Gene Q qPCR instrument (QIAGEN). The temperature was ramped from 25° C. to 90° C. with 6° C. increase per minute. The gain was set to the first sample in the run which was always a protein without ligand that was used as a reference. CPM dye binding was monitored using 365 nm excitation and 460 nm emission. The data was analyzed with the instrument software and the melting temperatures were calculated as an average of each duplicate (duplicates on averages did not differ by more than 0.5° C.). Increase in the melting temperature (ΔTₘ) i.e. thermal shift value for each compound was calculated by subtracting the melting temperate of the apo receptor from the melting temperature of the receptor bound to a respective compound. The results are shown below in Table 2.

TABLE 2
cAMP Binding Affinities of Selected Compounds
as measured by Thermostability Assay

| | Human A_{2B} ΔTₘ | Human A_{2A} ΔTₘ |
|---|---|---|
| Compound | CPM (10 μM) | CPM (10 μM) |
| 1 | 16.6 | — |
| 2 | 12.5 | 0.3 |
| 3 | 13.0 | 2.5 |
| 4 | 12.3 | 0.5 |
| 5 | 11.5 | 0.75 |
| 6 | 13.3 | 1.7 |
| 7 | — | 2.8 |
| 8 | — | 0 |
| 9 | 7.3 | 1.8 |
| 10 | 4.4 | −0.2 |
| 11 | 6.3 | −0.1 |
| 12 | 12.2 | 1.1 |
| 13 | 11.6 | −0.35 |
| 14 | 12.9 | 0.2 |
| 15 | 4.3 | −0.25 |
| 16 | 9.2 | 0.1 |
| 17 | 7.8 | 0 |
| 18 | 7.6 | 0 |
| 22 | 15.8 | — |
| 23 | 6.8 | — |
| 24 | 9.3 | — |
| 25 | 8.8 | — |
| 26 | 10.8 | — |
| 27 | 12.1 | — |
| 28 | 6.6 | — |
| 29 | 13.5 | — |

1. A compound of Formula I: 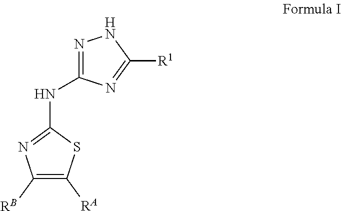
wherein:
R¹ is H, halogen, —C₁-C₈ alkyl, —C₃-C₈ cycloalkyl, —O—C₁-C₈ alkyl, or —CN, wherein each alkyl, cycloalkyl, or —O-alkyl is optionally substituted with one or more R²;
R² is independently, at each occurrence, selected from halogen, —OH, and —OC₁-C₈ alkyl;
R^{A} is a 5- to 6-membered heteroaryl, wherein said heteroaryl is optionally substituted with one or more R^{A1};
R^{A1} is independently, at each occurrence, selected from halogen, —OH, oxo, cyano, —C₁-C₈ alkyl, —C₃-C₈ cycloalkyl, and —O—C₁-C₈ alkyl, wherein each alkyl, cycloalkyl, or —O-alkyl is optionally substituted with one or more R^{A2};
R^{A2} is independently, at each occurrence, halogen, —OC₁-C₈ alkyl, or —OH;
R^{B} is phenyl or a 5- to 6-membered heteroaryl, wherein each phenyl or heteroaryl is optionally substituted with one or more R^{B1}; and
R^{B1} is independently, at each occurrence, selected from halogen, —OH, cyano, —C₁-C₈ alkyl, and —O—C₁-C₈ alkyl, wherein each alkyl and —O-alkyl is optionally substituted with one or more R^{B2}; and
R^{B2} is independently, at each occurrence, halogen, —OH, or —OC₁-C₈ alkyl;
or a pharmaceutically acceptable salt, solvate, or hydrate thereof.

2. The compound of claim 1, wherein R¹ is H, halogen, —C₁-C₈ alkyl, —C₃-C₈ cycloalkyl, or —OC₁-C₈ alkyl, wherein each alkyl, cycloalkyl, or —O-alkyl is optionally substituted with one or more R².

3. The compound of any of claims 1-2, wherein R² is —OH, halogen or —OC₁-C₄ alkyl.

4. The compound of any of the preceding claims, wherein R^{A} is imidazolyl, triazolyl, isoxazolyl, oxazolyl, pyrazolyl, pyridinyl, pyridazinyl, pyrimidinyl, or pyrazinyl; wherein each imidazolyl, triazolyl, isoxazolyl, oxazolyl, pyrazolyl, pyridinyl, pyridazinyl, pyrimidinyl, or pyrazinyl is optionally substituted with one or more R^{A1}.

5. The compound of any of the preceding claims, wherein R^{A1} is independently halogen, —OH, —C₁-C₄ alkyl, or —C₃-C₆ cycloalkyl, wherein each alkyl or cycloalkyl is optionally substituted with one or more halogen or —OH.

6. The compound of any of the preceding claims, wherein R^{A} is selected from: 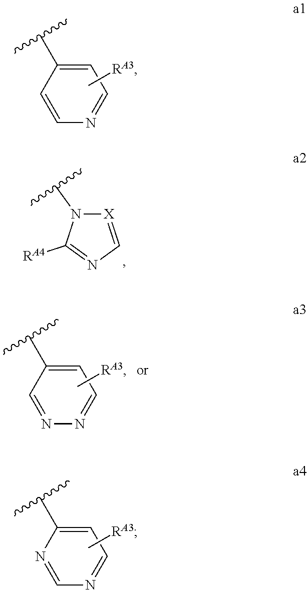
wherein
X is —CH— or N;
R^{A3} is independently, at each occurrence, 1 to 3 identical or different substituents selected from hydrogen, halogen, —C₁-C₈ alkyl, —C₁-C₈ haloalkyl, —C₁-C₈ alkoxy, —C₁-C₈ hydroxyalkyl, and —C₁-C₈ alkoxyalkyl; and
R^{A4} is independently, at each occurrence, hydrogen, halogen, —C₁-C₈ alkyl, —C₃-C₈ cycloalkyl, —C₁-C₈ hydroxyalkyl, and —C₁-C₈ alkoxyalkyl.

7. The compound of any of the preceding claims, wherein R^{B} is selected from phenyl, oxazolyl, pyridinyl, pyrimidinyl, pyridazinyl, and pyrazinyl, wherein each phenyl, oxazolyl, pyridinyl, pyrimidinyl, pyridazinyl, and pyrazinyl is optionally substituted by one or more R^{B1}.

8. The compound of any of the preceding claims, wherein R^{B1} is selected from halogen, —OH, cyano, —C₁-C₄ alkyl, and —C₁-C₄ alkoxy, wherein each alkyl and alkoxy is independently substituted with one or more halogen, —OH, or —OC₁-C₄ alkoxy.

9. The compound of any of the preceding claims, wherein R^{B} is selected from: 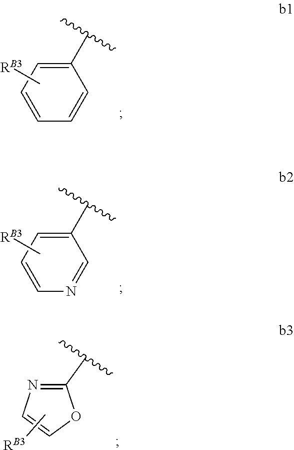
wherein
R^{B3} is independently, at each occurrence, selected from hydrogen, halogen, —C₁-C₈ alkyl, —C₁-C₈ haloalkyl, —C₁-C₈ alkoxy, —C₁-C₈ hydroxyalkyl, —C₁-C₈ alkoxyalkyl, and —CN.

10. The compound of any of the preceding claims, wherein R^{B} is phenyl substituted with one or two R^{B1}, wherein R^{B1} is independently, at each occurrence, cyano or fluoro.

11. The compound of any of the preceding claims, wherein the compound is of the Formula Ia3b or Ia2b: 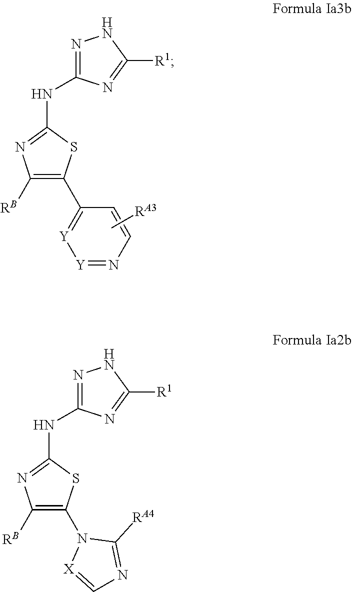
wherein:
R¹ is H, halogen, —C₁-C₈ alkyl, —C₃-C₈ cycloalkyl, —O—C₁-C₈ alkyl, or —CN, wherein each alkyl, cycloalkyl, or —O-alkyl is optionally substituted with one or more R²;
R² is independently, at each occurrence, selected from halogen, —OH, and —OC₁-C₈ alkyl;
X is —CH— or N;
Y is independently, at each occurrence, —CH— or N, wherein at least one of Y is —CH—;
R^{A3} is independently, at each occurrence, 1 to 3 identical or different substituents selected from hydrogen, halogen, —C₁-C₈ alkyl, —C₁-C₈ haloalkyl, —C₁-C₈ alkoxy, —C₁-C₈ hydroxyalkyl, and —C₁-C₈ alkoxyalkyl;
R^{A4} is independently, at each occurrence, hydrogen, halogen, —C₁-C₈ alkyl, —C₃-C₈ cycloalkyl, —C₁-C₈ hydroxyalkyl, and —C₁-C₈ alkoxyalkyl;
R^{B} is phenyl or a 5- to 6-membered heteroaryl, wherein each phenyl or heteroaryl is optionally substituted with one or more R^{B1};
R^{B1} is independently, at each occurrence, selected from halogen, —OH, cyano, —C₁-C₈ alkyl, and —O—C₁-C₈ alkyl, wherein each alkyl and —O-alkyl is optionally substituted with one or more R^{B2}; and
R^{B2} is independently, at each occurrence, halogen, —OH, or —OC₁-C₈ alkyl.

12. The compound of any of the preceding claims, wherein the compound is of the Formula Ia1b1, Ia1b2, Ia1b3, Ia2b1, Ia2b2, Ia2b3, Ia3b1, Ia3b2, or Ia3b3: 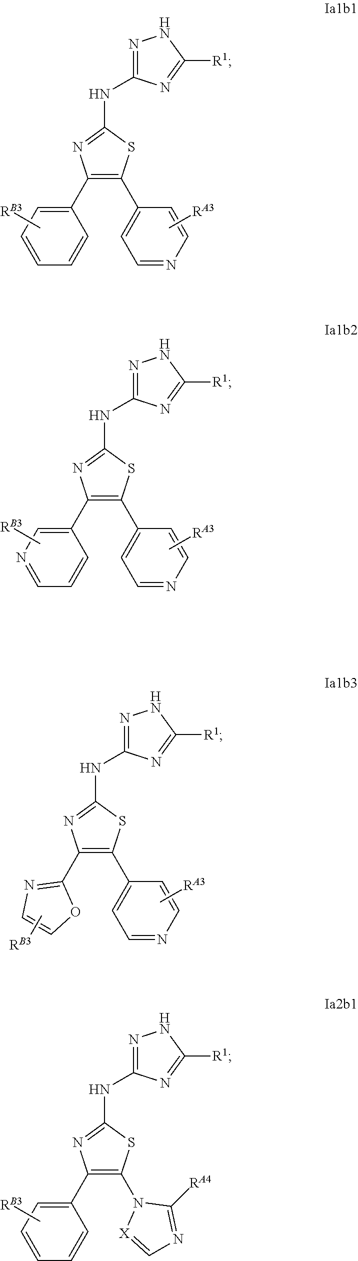 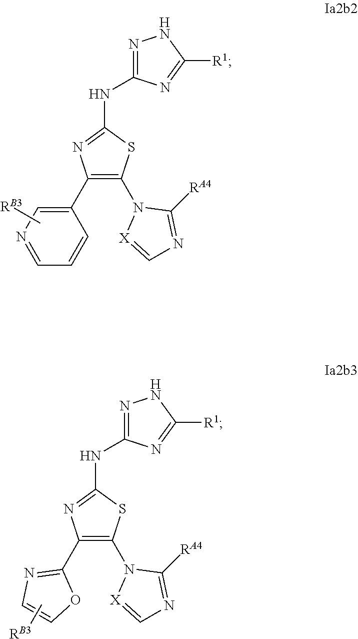 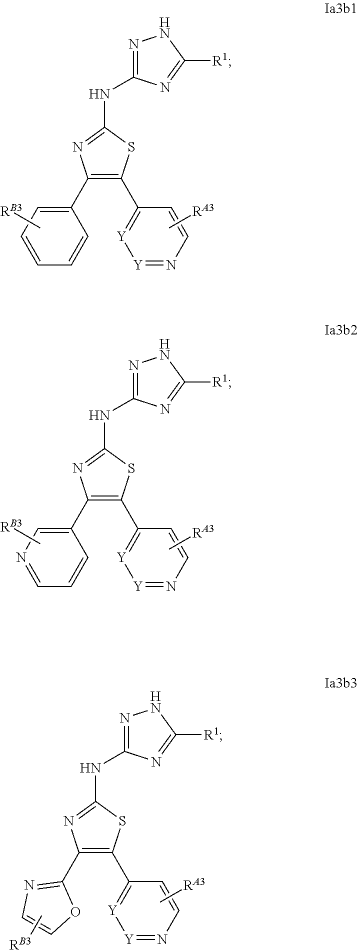
wherein:
R¹ is H, halogen, —C₁-C₈ alkyl, —C₃-C₈ cycloalkyl, —O—C₁-C₈ alkyl, or —CN, wherein each alkyl, cycloalkyl, or —O-alkyl is optionally substituted with one or more R²;
R² is independently, at each occurrence, selected from halogen, —OH, and —OC₁-C₈ alkyl;
X is —CH— or N;
Y is independently, at each occurrence, —CH— or N, wherein at least one of Y is —CH—;
R^{A3} is independently, at each occurrence, 1 to 3 identical or different substituents selected from hydrogen, halogen, —C₁-C₈ alkyl, —C₁-C₈ haloalkyl, —C₁-C₈ alkoxy, —C₁-C₈ hydroxyalkyl, and —C₁-C₈ alkoxyalkyl;
R^{A4} is independently, at each occurrence, hydrogen, halogen, —C₁-C₈ alkyl, —C₃-C₈ cycloalkyl, —C₁-C₈ hydroxyalkyl, and —C₁-C₈ alkoxyalkyl; and
R^{B3} is independently, at each occurrence, selected from hydrogen, halogen, —C₁-C₈ alkyl, —C₁-C₈ haloalkyl, —C₁-C₈ alkoxy, —C₁-C₈ hydroxyalkyl, —C₁-C₈ alkoxyalkyl, and —CN.

13. The compound of claim 1, wherein said compound is selected from:
5-cyclobutyl-N-[4-(2-fluorophenyl)-5-(3-fluoropyridin-4-yl)-1,3-thiazol-2-yl]-1H-1,2,4-triazol-3-amine;
3-[5-(3-chloropyridin-4-yl)-2-[(5-cyclopropyl-1H-1,2,4-triazol-3-yl)amino]-1,3-thiazol-4-yl]benzonitrile;
3-{2-[(5-cyclopropyl-1H-1,2,4-triazol-3-yl)amino]-5-(3-fluoropyridin-4-yl)-1,3-thiazol-4-yl}benzonitrile;
3-{2-[(5-cyclopropyl-1H-1,2,4-triazol-3-yl)amino]-5-(2,5-dimethylpyridin-4-yl)-1,3-thiazol-4-yl}benzonitrile;
3-{2-[(5-cyclopropyl-1H-1,2,4-triazol-3-yl)amino]-5-(3-methylpyridin-4-yl)-1,3-thiazol-4-yl}benzonitrile;
3-{2-[(5-cyclopropyl-1H-1,2,4-triazol-3-yl)amino]-5-(2,3-dimethylpyridin-4-yl)-1,3-thiazol-4-yl}benzonitrile;
3-{2-[(5-cyclopropyl-1H-1,2,4-triazol-3-yl)amino]-5-(2-methyl-1H-imidazol-1-yl)-1,3-thiazol-4-yl}benzonitrile;
3-{2-[(5-cyclopropyl-1H-1,2,4-triazol-3-yl)amino]-5-(2-methylpyridin-4-yl)-1,3-thiazol-4-yl}benzonitrile;
3-[5-(2-methyl-1H-imidazol-1-yl)-2-[(1H-1,2,4-triazol-3-yl)amino]-1,3-thiazol-4-yl]benzonitrile;
3-{2-[(5-methyl-1H-1,2,4-triazol-3-yl)amino]-5-(2-methyl-1H-imidazol-1-yl)-1,3-thiazol-4-yl}benzonitrile;
3-(2-{[5-(hydroxymethyl)-1H-1,2,4-triazol-3-yl]amino}-5-(2-methyl-1H-imidazol-1-yl)-1,3-thiazol-4-yl)benzonitrile;
3-[5-(2,5-dimethylpyridin-4-yl)-2-[(1H-1,2,4-triazol-3-yl)amino]-1,3-thiazol-4-yl]benzonitrile;
3-[5-(2,5-dimethylpyridin-4-yl)-2-[(5-methyl-1H-1,2,4-triazol-3-yl)amino]-1,3-thiazol-4-yl]benzonitrile;
3-[5-(2,5-dimethylpyridin-4-yl)-2-{[5-(hydroxymethyl)-1H-1,2,4-triazol-3-yl]amino}-1,3-thiazol-4-yl]benzonitrile;
3-{2-[(5-cyclopropyl-1H-1,2,4-triazol-3-yl)amino]-5-(1H-1,2,4-triazol-1-yl)-1,3-thiazol-4-yl}benzonitrile;
3-[5-(2-ethyl-1H-imidazol-1-yl)-2-[(5-methyl-1H-1,2,4-triazol-3-yl)amino]-1,3-thiazol-4-yl]benzonitrile;
3-{2-[(5-cyclopropyl-1H-1,2,4-triazol-3-yl)amino]-5-[2-(hydroxymethyl)-1H-imidazol-1-yl]-1,3-thiazol-4-yl}benzonitrile;
3-{2-[(5-cyclopropyl-1H-1,2,4-triazol-3-yl)amino]-5-(2-methyl-1H-imidazol-1-yl)-1,3-thiazol-4-yl}-4-fluorobenzonitrile;
3-{5-[2-(hydroxymethyl)-1H-imidazol-1-yl]-2-[(1H-1,2,4-triazol-3-yl)amino]-1,3-thiazol-4-yl}benzonitrile;
3-{2-[(5-ethyl-1H-1,2,4-triazol-3-yl)amino]-5-[2-(hydroxymethyl)-1H-imidazol-1-yl]-1,3-thiazol-4-yl}benzonitrile;
3-{5-[2-(hydroxymethyl)-1H-imidazol-1-yl]-2-[(5-methyl-1H-1,2,4-triazol-3-yl)amino]-1,3-thiazol-4-yl}benzonitrile;
3-[5-(3-methylpyridin-4-yl)-2-[(1H-1,2,4-triazol-3-yl)amino]-1,3-thiazol-4-yl]benzonitrile;
3-{2-[(5-cyclopropyl-1H-1,2,4-triazol-3-yl)amino]-5-(5-methylpyridazin-4-yl)-1,3-thiazol-4-yl}benzonitrile;
3-{2-[(5-cyclopropyl-1H-1,2,4-triazol-3-yl)amino]-5-(pyrimidin-4-yl)-1,3-thiazol-4-yl}benzonitrile;
3-{2-[(5-cyclopropyl-1H-1,2,4-triazol-3-yl)amino]-5-(pyridazin-4-yl)-1,3-thiazol-4-yl}benzonitrile;
3-{2-[(5-cyclopropyl-1H-1,2,4-triazol-3-yl)amino]-5-(pyridin-4-yl)-1,3-thiazol-4-yl}benzonitrile;
3-{2-[(5-cyclopropyl-1H-1,2,4-triazol-3-yl)amino]-5-(5-methylpyrimidin-4-yl)-1,3-thiazol-4-yl}benzonitrile;
m-[2-(5-cyclopropyl-1H-1,2,4-triazol-3-ylamino)-5-(2-ethyl-1-imidazolyl)-1,3-thiazol-4-yl]benzonitrile; and
3-{2-[(5-cyclopropyl-1H-1,2,4-triazol-3-yl)amino]-5-(1-methyl-6-oxo-1,6-dihydropyridin-3-yl)-1,3-thiazol-4-yl}benzonitrile.

14. A pharmaceutical composition comprising a compound according to any of the preceding claims and a pharmaceutically acceptable diluent or carrier.

15. A compound of any of claims 1-13 or a pharmaceutical composition of claim 14 for use as a medicament, preferably for use in a method of treating a condition, disorder or disease mediated by activation of the adenosine A_{2B} receptor.
